(19) United States
(12) Patent Application Publication
(10) Pub. No.: US 2024/0285614 A1
(43) Pub. Date: Aug. 29, 2024

(54) PHENOL RIBONUCLEOTIDE REDUCTASE (RNR) INHIBITORS AND USES THEREOF

(71) Applicant: Boundless Bio, Inc., San Diego, CA (US)

(72) Inventors: Anthony B. PINKERTON, Rancho Santa Fe, CA (US); Jacques MAUGER, San Diego, CA (US); Yen Pham Hong TRUONG, San Diego, CA (US); Camille REMEUR, Toulouse (FR); Jérôme TOUM, Toulouse (FR); Olivier MIRGUET, Toulouse (FR)

(21) Appl. No.: 18/433,734

(22) Filed: Feb. 6, 2024

(30) Foreign Application Priority Data
Feb. 7, 2023 (FR) 2301153

(51) Int. Cl. A61K 31/496; A61K 31/404; A61K 31/435; A61K 31/438; A61K 31/454; A61K 31/4995; A61K 45/06; C07D 207/12; C07D 209/08; C07D 401/12; C07D 401/14; C07D 403/12; C07D 471/04; C07D 471/08; C07D 471/10; C07D 487/08

(52) U.S. Cl. CPC A61K 31/496; A61K 31/404; A61K 31/435; A61K 31/438; A61K 31/454; A61K 31/4995; A61K 45/06; C07D 207/12; C07D 209/08; C07D 401/12; C07D 401/14; C07D 403/12; C07D 471/04; C07D 471/08; C07D 471/10; C07D 487/08

(57) ABSTRACT

Provided herein are compounds and methods for the treatment of cancer. The methods include administering to a subject in need a therapeutically effective amount of a cyclic sulfonamide RNR inhibitor disclosed herein.

### CROSS-REFERENCE

This patent application claims the benefit of French Application No. 2301153, filed Feb. 7, 2023; which is incorporated herein by reference in its entirety.

### FIELD OF THE INVENTION

Described herein are compounds, methods of making such compounds, pharmaceutical compositions, and medicaments comprising such compounds, and methods of using such compounds for inhibiting ribonucleotide reductase (RNR).

### BACKGROUND OF THE INVENTION

Ribonucleotide reductase (RNR), also known as ribonucleotide diphosphate reductase (rNDP), is composed of a hetero-oligomer of a large subunit M1 and a small subunit M2, and expression of both is required for enzyme activity. RNR is a highly regulated enzyme in the deoxyribonucleotide synthesis pathway that is ubiquitously present in human, bacteria, yeast, and other organisms RNR is responsible for the de novo conversion of ribonucleotide diphosphate to 2′-deoxyribonucleotide diphosphate, a process that is essential for DNA synthesis and repair. RNR is directly involved in DNA synthesis and repair, tumor growth, metastasis, and drug resistance. In various types of solid tumors and blood cancers, numerous correlations have been reported with overexpression of M2 and their prognosis. In addition, cell growth inhibition by inhibiting RNR and anti-tumor effect in vivo have been reported in cell lines derived from several cancer types and in nonclinical models.

The proliferation of cancer cells requires excess deoxyribonucleotide triphosphates (dNTPs) for DNA synthesis. Therefore, an increase in RNR activity is necessary as it helps provide extra dNTPs for DNA replication in primary and metastatic cancer cells. Because of this critical role in DNA synthesis, RNR represents an important target for cancer therapy. However, existing chemotherapies that target RNR are nucleoside-based analogs. Hence, they are promiscuous, leading to nonspecific binding of other nucleoside binding proteins which results in unwanted side effects. Therefore, there is a need for compositions and methods for specifically targeting and inhibiting RNR activity in neoplastic cells in the treatment of cancer.

### BRIEF SUMMARY OF THE INVENTION

Described herein are RNR inhibitors that are useful in treating cancer.

Disclosed herein is a compound of Formula (I), or a pharmaceutically acceptable salt, solvate, or stereoisomer thereof:

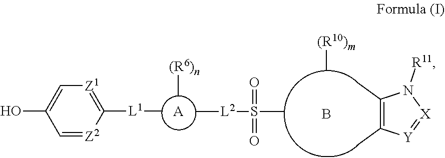

wherein:
Z¹ is N or CR¹;
R¹ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Z² is N or CR²;
R² is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent, —C(R³R⁴)—, —O—, —S—, —NR⁵—, —C(R³R⁴)—O—, —O—C(R³R⁴)—, —C(R³R⁴)—S—, —S—C(R³R⁴)—, —C(R³R⁴)—NR⁵—, or —NR⁵—C(R³R⁴)—;
R³ and R⁴ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁵ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl);
Ring A is cycloalkyl, heterocycloalkyl, aryl, or heteroaryl;
each R⁶ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkyl, cycloalkyl, or heterocycloalkyl;
n is 0, 1, 2, 3, or 4;
L² is absent, —CR⁷R⁸—, or —NR⁹—;
R⁷ and R⁸ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁹ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁—C deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl);
Ring B is phenyl or a 6-membered heteroaryl;
each R¹⁶ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
m is 0, 1, 2, or 3;
R¹¹ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X is N or CR^{X};
R^{X} is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆ heteroalkyl, C₂-C₆alkenyl, C₂-C₆alkynyl, cycloalkyl, or heterocycloalkyl;
Y is N or CR^{Y};
R^{Y} is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆ alkenyl, C₂-C₆alkynyl, cycloalkyl, or heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, any, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆ haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl. C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₆alkyl)₂, —N═S(═O)(C₁-C₃alkyl)₂, —C(═O)C₁-C₆alkyl, —C(═O)OH, —C(═O)OC₁-C₃alkyl, —C(═O)NH₂, —C(═O)NHC₁-C₆alkyl, —C(═O)N(C₁-C₃alkyl)₂, —P(═O)(C₁-C₆alkyl), C₁-C₆alkyl, C₁-C₃alkoxy, C₁-C₆haloalkyl, C₁-C₃haloalkoxy, C₁-C₃hydroxyalkyl, C₁-C₃aminoalkyl, C₁-C₃heteroalkyl, 3- to 6-membered cycloalkyl, or 3- to 6-membered heterocycloalkyl;
or two R on the same atom are taken together to form an oxo;
   provided that the compound is not

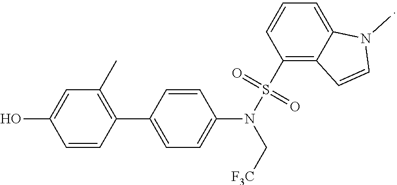 [SMILES: Cc1cc(O)ccc1-c1ccc(N(CC(F)(F)F)S(=O)(=O)c2cccc3c2ccn3C)cc1]

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating cancer in a subject, comprising administering to the subject a compound disclosed herein, or a pharmaceutically acceptable salt, solvate, or stereoisomer thereof, or a pharmaceutical composition disclosed herein.

Also disclosed herein is a method of inhibiting ribonucleotide reductase in a subject, comprising administering to the subject a compound disclosed herein, or a pharmaceutically acceptable salt, solvate, or stereoisomer thereof, or a pharmaceutical composition disclosed herein.

In some embodiments, the inhibition of ribonucleotide reductase occurs in a tumor cell in the subject in need thereof.

Also disclosed herein is a method for treating a tumor or tumor cells in a subject, the method comprising administering a compound disclosed herein, or a pharmaceutically acceptable salt, solvate, or stereoisomer thereof, in an amount sufficient to induce replication stress in the tumor or tumor cells; and administering a cancer-targeted therapeutic agent; wherein the tumor or tumor cells have an ecDNA signature; and wherein growth or size of the tumor or growth or number of tumor cells is reduced.

Also disclosed herein is a method of treating an ecDNA-associated tumor or tumor cells comprising administering a compound disclosed herein, or a pharmaceutically acceptable salt, solvate, or stereoisomer thereof, to a subject identified as having a tumor or tumor cells having ecDNA, wherein growth or size of the tumor or growth or number of the tumor cells is decreased as a result of treatment.

In some embodiments, the method further comprises administering a cancer-targeted therapeutic agent.

In some embodiments, the cancer-targeted therapeutic agent inhibits a gene or gene product comprised on ecDNA in the tumor or tumor cells.

Also disclosed herein is a method for treating a tumor or tumor cells in a subject, the method comprising administering a compound disclosed herein, or a pharmaceutically acceptable salt, solvate, or stereoisomer thereof, in an amount sufficient to induce replication stress in the tumor or tumor cells, wherein the tumor or tumor cells comprises ecDNA or have an ecDNA signature; and wherein growth or size of the tumor or growth or number of tumor cells is reduced.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an.” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Oxo” refers to ═O.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen. In some embodiments, the alkyl is optionally substituted with —COOH, —COOMe, —CONH₂, —CONHMe, or —CONMe₂.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₁-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₇, alkenyl, a C₂—C alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen. In some embodiments, the alkenyl is optionally substituted with —COOH, —COOMe, —CONH₂, —CONHMe, or —CONMe₂.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-alkynyl” means that the alkynyl group may consist of 2 carbon atoms, 3 carbon atoms, 4 carbon atoms, 5 carbon atoms or 6 carbon atoms, although the present definition also covers alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. In some embodiments, the alkynyl is optionally substituted with —COOH, —COOMe, —CONH₂, —CONHMe, or —CONMe₂.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is optionally substituted with —COOH, —COOMe, —CONH₂, —CONHMe, or —CONMe₂.

“Alkoxy” refers to a radical of the formula —Oalkyl where alkyl is as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen. In some embodiments, the alkoxy is optionally substituted with —COOH, —COOMe, —CONH₂, —CONHMe, or —CONMe₂.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen. In some embodiments, the aryl is optionally substituted with —COOH, —COOMe, —CONH₂, —CONHMe, or —CONMe₂.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. In some embodiments, the cycloalkyl is optionally substituted with —COOH, —COOMe, —CONH₂, —CONHMe, or —CONMe₂.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro. In some embodiments, halogen is chloro. In some embodiments, halogen is bromo. In some embodiments, halogen is iodo.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. In some embodiments, the heteroalkyl is optionally substituted with —COOH, —COOMe, —CONH₂, —CONHMe, or —CONMe₂.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a 3- to 24-membered partially or fully saturated, not fully aromatic ring radical comprising 2 to 23 carbon atoms and from one to 8 heteroatoms selected from the group consisting of nitrogen, oxygen, phosphorous and sulfur. In some embodiments, the heterocycloalkyl comprises 1 or 2 heteroatoms selected from nitrogen and oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₈ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen. In some embodiments, the heterocycloalkyl is optionally substituted with —COOH, —COOMe, —CONH₂, —CONHMe, or —CONMe₂.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring comprising at least one heteroatom. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazol[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen. In some embodiments, the heteroaryl is optionally substituted with —COOH, —COOMe, —CONH₂, —CONHMe, or —CONMe₂.

The term “one or more” when referring to an optional substituent means that the subject group is optionally substituted with one, two, three, four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The terms “treat,” “treated,” “treatment,” or “treating” as used herein refers to therapeutic treatment, wherein the object is to slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease, delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “ecDNA signature” as used herein, generally refers to one or more characteristics common to tumors or tumor cells that are ecDNA+ (contain extrachromosomal DNA (ecDNA)). In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, ecDNA signature includes a detection or identification of ecDNA using an imaging technology. In some cases, ecDNA signature does not include any imaging or direct detection of ecDNA.

### Compounds

Described herein are cyclic sulfonamide RNR inhibitors that are useful for the treatment of cancer.

Disclosed herein is a compound of Formula (I), or a pharmaceutically acceptable salt, solvate, or stereoisomer thereof:

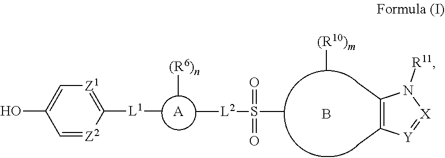

wherein:
Z¹ is N or CR¹:
R¹ is hydrogen, deuterium, halogen, —CN, —OH, —OR, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Z² is N or CR²;
R² is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₁-C₆alkenyl, C₁-C₆alkynyl, cycloalkyl, or heterocycloalkyl;
L¹ is absent, —C(R³R⁴)—, —O—, —S—, —NR⁵—, —C(R³R⁴)—O—, —O—C(R³R⁴)—, —C(R³R⁴)—S—, —S—C(R³R⁴)—, —C(R³R⁴)—NR⁵—, or —NR⁵—C(R³R⁴)—;
R³ and R⁴ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₁-C₆alkenyl, C₁-C₆alkynyl, cycloalkyl, or heterocycloalkyl;
R⁵ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl);
Ring A is cycloalkyl, heterocycloalkyl, aryl, or heteroaryl;
each R⁶ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
n is 0, 1, 2, 3, or 4;
L² is absent, —CR⁷R⁸—, or —NR⁹—;
R⁷ and R⁸ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁹ is hydrogen, —S(═O)R^{a}, —S(═O)₂R^{a}, —S(═O)₂NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₁-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl);
Ring B is phenyl or a 6-membered heteroaryl;
each R¹⁰ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
m is 0, 1, 2, or 3;
R¹¹ is hydrogen, —S(═O)R^{a}, —S(═O)₂R^{a}, —S(═O)₂NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl:
X is N or CR^{X};
R^{X} is hydrogen, deuterium, halogen, —CN, —OH, —OR, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is N or CR^{X};
R^{X} is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C_{f}alkyl, C₁-C₆haloalkyl, C₁-C₆deuteroalkyl, C₁-C₆hydroxyalkyl, C₁-C₆aminoalkyl, C₁-C₆heteroalkyl, C₁-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-Cₜalkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₆alkyl, —N(C₁-C₃alkyl)₂, —N═S(═O)(C₁-C₃alkyl)₂, —C(═O)C₁-C₆alkyl, —C(═O)OH, —C(═O)OC₁-C₃alkyl, —C(═O)NH₂, —C(═O)NHC₁-C₃alkyl, —C(═O)N(C₁-C₆alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 3- to 6-membered cycloalkyl, or 3- to 6-membered heterocycloalkyl;
or two R on the same atom are taken together to form an oxo;
provided that the compound is not:

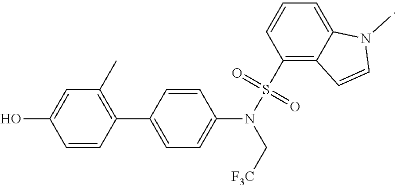 [SMILES: Cc1cc(O)ccc1-c1ccc(N(CC(F)(F)F)S(=O)(=O)c2cccc3c2ccn3C)cc1]

In some embodiments of a compound of Formula (I), Z¹ is N. In some embodiments of a compound of Formula (I). Z¹ is CR¹.

In some embodiments of a compound of Formula (I), R¹ is hydrogen, deuterium, halogen, —CN, —OH, —OR^{a}, —NR^{c}R^{d}, C₁-C₆alkyl, or C₁-C_{f}haloalkyl. In some embodiments of a compound of Formula (I), R¹ is hydrogen, halogen, C₁-C₆alkyl, or C₁-C₆haloalkyl. In some embodiments of a compound of Formula (I), R¹ is hydrogen, halogen, or C₁-C₆alkyl. In some embodiments of a compound of Formula (I), R¹ is hydrogen or halogen. In some embodiments of a compound of Formula (I), R¹ is hydrogen or C₁-C₆alkyl. In some embodiments of a compound of Formula (I), R¹ is hydrogen.

In some embodiments of a compound of Formula (I), Z² is N. In some embodiments of a compound of Formula (I), Z² is CR¹.

In some embodiments of a compound of Formula (I), R² is hydrogen, deuterium, halogen, —CN, —OH, —OR^{a}, —NR^{c}R^{d}, C₁-C₆alkyl, or C₁-C_{f}haloalkyl. In some embodiments of a compound of Formula (I), R² is hydrogen, halogen, C₁-C₆alkyl, or C₁-C₆haloalkyl. In some embodiments of a compound of Formula (I), R² is hydrogen, halogen, or C₁-C₆alkyl. In some embodiments of a compound of Formula (I), R² is hydrogen or halogen. In some embodiments of a compound of Formula (I), R² is hydrogen or C₁-C₆alkyl. In some embodiments of a compound of Formula (I), R² is hydrogen.

In some embodiments of a compound of Formula (I), X is N. In some embodiments of a compound of Formula (I), X is CR¹.

In some embodiments of a compound of Formula (I), R¹ is hydrogen, deuterium, halogen, —CN, —OH, —OR^{a}, —NR^{c}R^{d}, C₁-C₆alkyl, or C₁-C₆haloalkyl. In some embodiments of a compound of Formula (I), R^{X} is hydrogen, halogen, C₁-C₆alkyl, or C₁-C₆haloalkyl. In some embodiments of a compound of Formula (I), R^{X} is hydrogen, halogen, or C₁-C₆alkyl. In some embodiments of a compound of Formula (I), R^{X} is hydrogen or halogen. In some embodiments of a compound of Formula (I), R^{X} is hydrogen or C₁-C₆alkyl. In some embodiments of a compound of Formula (I), R^{X} is hydrogen.

In some embodiments of a compound of Formula (I), Y is N. In some embodiments of a compound of Formula (I), Y is CR^{Y}.

In some embodiments of a compound of Formula (I), R^{Y} is hydrogen, deuterium, halogen, —CN, —OH, —OR^{a}, —NR^{c}R^{d}, C₁-C₆alkyl, or C₁-C₆haloalkyl. In some embodiments of a compound of Formula (I), R^{Y} is hydrogen, halogen, C₁-C₆alkyl, or C₁-C₆haloalkyl. In some embodiments of a compound of Formula (I), R^{Y} is hydrogen, halogen, or C₁-C₆alkyl. In some embodiments of a compound of Formula (I), R is hydrogen or halogen. In some embodiments of a compound of Formula (I), R¹ is hydrogen or C₁-C₆alkyl. In some embodiments of a compound of Formula (I), R¹ is hydrogen.

In some embodiments of a compound of Formula (I), Ring B is phenyl. In some embodiments of a compound of Formula (I), Ring B is a 6-membered heteroaryl. In some embodiments of a compound of Formula (I), Ring B is pyridinyl.

In some embodiments of a compound of Formula (I), the compound is of Formula (Ia):

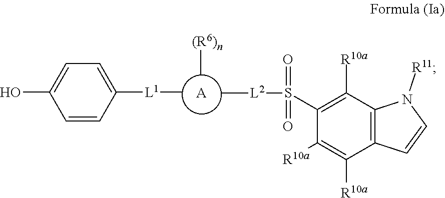

wherein each R^{10a} is independently hydrogen or R¹⁰.

In some embodiments of a compound of Formula (I), the compound is of Formula (Ib):

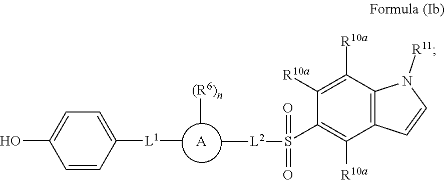

wherein each R^{10a} is independently hydrogen or R¹⁰.

In some embodiments of a compound of Formula (I), the compound is of Formula (Ic):

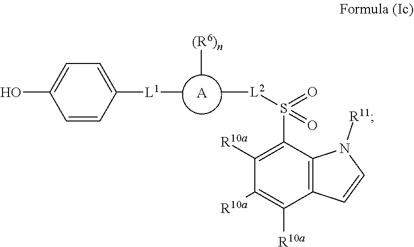

wherein each R^{10a} is independently hydrogen or R¹⁰.

In some embodiments of a compound of Formula (I), the compound is of Formula (Id):

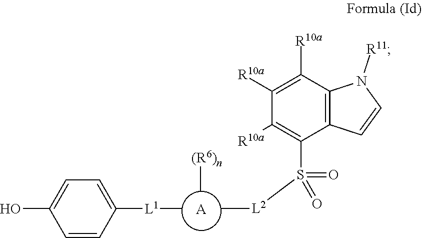

wherein each R^{10a} is independently hydrogen or R¹⁰.

In some embodiments of a compound of Formula (I) or (Ia)-(Id), each R¹⁰ is independently deuterium, halogen, —CN, —OH, —OR, —NR^{c}R^{d}, C₁-C₆alkyl, or C₁-C₆haloalkyl.

In some embodiments of a compound of Formula (I) or (Ia)-(Id), each R¹⁰ is independently deuterium, halogen, C₁-C₆alkyl, or C₁-C₆haloalkyl. In some embodiments of a compound of Formula (I) or (Ia)-(Id), each R¹⁰ is independently halogen. C₁-C₆alkyl, or C₁-C₆haloalkyl. In some embodiments of a compound of Formula (I) or (Ia)-(Id), each R¹⁰ is independently halogen.

In some embodiments of a compound of Formula (I), m is 0 or 1. In some embodiments of a compound of Formula (I), m is 0-2. In some embodiments of a compound of Formula (I), m is 1 or 2. In some embodiments of a compound of Formula (I), m is 1. In some embodiments of a compound of Formula (I), m is 2. In some embodiments of a compound of Formula (I), m is 3.

In some embodiments of a compound of Formula (Ia)-(Id), each R^{10a} is independently hydrogen, deuterium, halogen, —CN, —OH, —OR^{a}, —NR^{c}R^{d}, C₁-C₆alkyl, or C₁-C₆haloalkyl. In some embodiments of a compound of Formula (Ia)-(Id), each R^{10a} is independently hydrogen, deuterium, halogen, C₁-C₆alkyl, or C₁-C₆haloalkyl. In some embodiments of a compound of Formula (Ia)-(Id), each R^{10a} is independently hydrogen, halogen, C₁-C₆alkyl, or C₁-C₆haloalkyl. In some embodiments of a compound of Formula (Ia)-(Id), each R^{10a} is independently hydrogen or halogen. In some embodiments of a compound of Formula (Ia)-(Id), each R^{10a} is independently hydrogen.

In some embodiments of a compound of Formula (I) or (Ia)-(Id), L¹ is absent, —C(R³R⁴)—, —O—, —S—, —NR⁵—, —C(R³R⁴)—NR⁵—, or —NR⁵—C(R³R⁴)—. In some embodiments of a compound of Formula (I) or (Ia)-(Id), L¹ is absent, —O—, —NR⁵, or —NR⁵—C(R³R⁴)—. In some embodiments of a compound of Formula (I) or (Ia)-(Id), L¹ is absent. In some embodiments of a compound of Formula (I) or (Ia)-(Id), L¹ is —O—. In some embodiments of a compound of Formula (I) or (Ia)-(Id), L¹ is —NR⁵—. In some embodiments of a compound of Formula (I) or (Ia)-(Id), L¹ is —NR⁵—C(R³R⁴)—.

In some embodiments of a compound of Formula (I) or (Ia)-(Id), R³ and R⁴ are independently hydrogen, deuterium, halogen, —CN, —OH, —OR^{a}, —NR^{c}R^{d}, C₁-C₆alkyl, or C₁-C₆haloalkyl. In some embodiments of a compound of Formula (I) or (Ia)-(Id), R³ and R⁴ are independently hydrogen, halogen, C₁-C₆alkyl, or C₁-C₆haloalkyl. In some embodiments of a compound of Formula (I) or (Ia)-(Id), R¹ and R⁴ are independently hydrogen, halogen, or C₁-C₆alkyl. In some embodiments of a compound of Formula (I) or (Ia)-(Id), R³ and R⁴ are independently hydrogen or halogen. In some embodiments of a compound of Formula (I) or (Ia)-(Id), R³ and R⁴ are independently hydrogen or C₁-C₆alkyl. In some embodiments of a compound of Formula (I) or (Ia)-(Id), R³ and R⁴ are hydrogen.

In some embodiments of a compound of Formula (I) or (Ia)-(Id), R⁵ is hydrogen, C₁-C₆alkyl, C₁-C₆haloalkyl, cycloalkyl, heterocycloalkyl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (I) or (Ia)-(Id), R⁵ is hydrogen, C₁-C₆alkyl, C₁-C₆haloalkyl, cycloalkyl, or heterocycloalkyl.

In some embodiments of a compound of Formula (I) or (Ia)-(Id), R is hydrogen or C₁-C₆alkyl. The compound of any one of claims 1-27 or 30-35, or a pharmaceutically acceptable salt, solvate, or stereoisomer thereof, wherein R⁵ is C₁-C₆alkyl.

In some embodiments of a compound of Formula (I) or (Ia)-(Id), L² is absent or —NR⁹—. In some embodiments of a compound of Formula (I) or (Ia)-(Id), L² is absent. In some embodiments of a compound of Formula (I) or (Ia)-(Id), L is NR⁹—. In some embodiments of a compound of Formula (I) or (Ia)-(Id), L² is —CR⁷R⁸—.

In some embodiments of a compound of Formula (I) or (Ia)-(Id), R⁷ and R⁸ are independently hydrogen, deuterium, halogen, —CN, —OH, —OR^{a}, —NR^{c}R^{d}, C₁-C₆alkyl, or C₁-C₆haloalkyl. In some embodiments of a compound of Formula (I) or (Ia)-(Id), R⁷ and R⁸ are independently hydrogen, halogen, C₁-C₆alkyl, or C₁-C₆haloalkyl. In some embodiments of a compound of Formula (I) or (Ia)-(Id), R⁷ and R⁸ are independently hydrogen, halogen, or C₁-C₆alkyl. In some embodiments of a compound of Formula (I) or (Ia)-(Id), R⁷ and R⁸ are independently hydrogen or halogen. In some embodiments of a compound of Formula (I) or (Ia)-(Id), R⁷ and R⁸ are independently hydrogen or C₁-C₆alkyl.

In some embodiments of a compound of Formula (I) or (Ia)-(Id), R⁹ is hydrogen, C₁-C₆alkyl, C₁-C₆haloalkyl, cycloalkyl, or heterocycloalkyl. In some embodiments of a compound of Formula (I) or (Ia)-(Id), R¹ is hydrogen, C₁-C₆alkyl, or C₁-C₆haloalkyl. In some embodiments of a compound of Formula (I) or (Ia)-(Id), R⁹ is hydrogen or C₁-C₆alkyl. In some embodiments of a compound of Formula (I) or (Ia)-(Id), R⁹ is hydrogen. In some embodiments of a compound of Formula (I) or (Ia)-(Id), R⁹ is C₁-C₆alkyl.

In some embodiments of a compound of Formula (I) or (Ia)-(Id), R¹¹ is hydrogen, C₁-C₆alkyl, or C₁-C₆haloalkyl. In some embodiments of a compound of Formula (I) or (Ia)-(Id), R¹¹ is hydrogen or C₁-C₆alkyl. In some embodiments of a compound of Formula (I) or (Ia)-(Id), R¹¹ is C₁-C₆alkyl. In some embodiments of a compound of Formula (I) or (Ia)-(Id), R¹¹ is hydrogen.

In some embodiments of a compound of Formula (I) or (Ia)-(Id), Ring A is cycloalkyl or heterocycloalkyl.

In some embodiments of a compound of Formula (I) or (Ia)-(Id), Ring A is heterocycloalkyl.

In some embodiments of a compound of Formula (I) or (Ia)-(Id), Ring A is monocyclic heterocycloalkyl. In some embodiments of a compound of Formula (I) or (Ia)-(Id), Ring A is 3- to 7-membered monocyclic heterocycloalkyl. In some embodiments of a compound of Formula (I) or (Ia)-(Id), Ring A is 4- to 6-membered monocyclic heterocycloalkyl. In some embodiments of a compound of Formula (I) or (a)-(Id), Ring A is 4-membered monocyclic heterocycloalkyl. In some embodiments of a compound of Formula (I) or (Ia)-(Id), Ring A is 5-membered monocyclic heterocycloalkyl. In some embodiments of a compound of Formula (I) or (Ia)-(Id), Ring A is 6-membered monocyclic heterocycloalkyl. In some embodiments of a compound of Formula (I) or (Ia)-(Id), Ring A is 7-membered monocyclic heterocycloalkyl.

In some embodiments of a compound of Formula (I) or (Ia)-(Id), Ring A

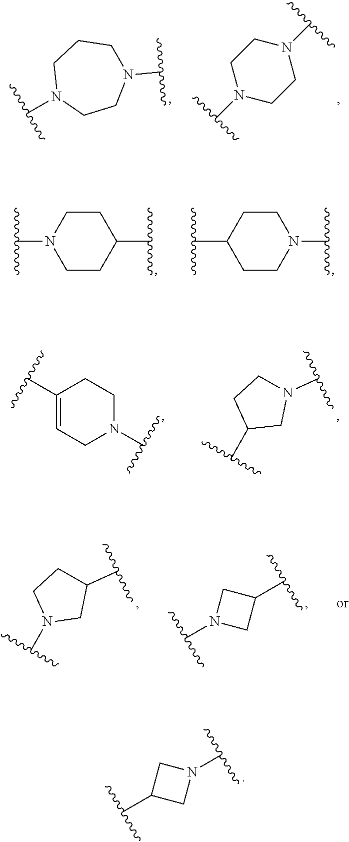

In some embodiments of a compound of Formula (I) or (Ia)-(Id), Ring A is,

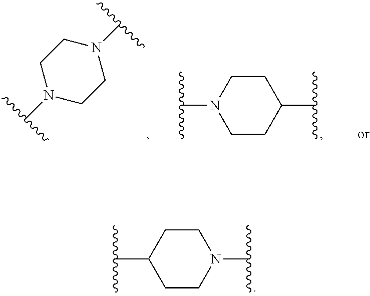 [SMILES: *N1CCN(*)CC1 ; *C1CCN(*)CC1 ; *C1CCN(*)CC1]

In some embodiments of a compound of Formula (I) or (Ia)-(Id), Ring A is

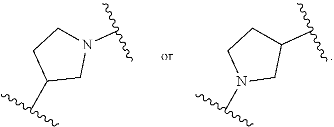 [SMILES: *C1CCN(*)C1 ; *C1CCN(*)C1]

In some embodiments of a compound of Formula (I) or (Ia)-(Id), Ring A is

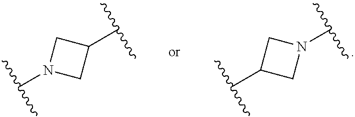 [SMILES: *C1CN(*)C1 ; *C1CN(*)C1]

In some embodiments of a compound of Formula (I) or (Ia)-(Id), Ring A is heterocycloalkyl. In some embodiments of a compound of Formula (I) or (Ia)-(Id), Ring A is 8- to 12-membered bicyclic heterocycloalkyl. In some embodiments of a compound of Formula (I) or (Ia)-(Id), Ring A is 8- to 10-membered bicyclic heterocycloalkyl. In some embodiments of a compound of Formula (I) or (Ia)-(Id), Ring A is a fused bicyclic heterocycloalkyl. In some embodiments of a compound of Formula (I) or (Ia)-(Id), Ring A is a bridged bicyclic heterocycloalkyl. In some embodiments of a compound of Formula (I) or (Ia)-(Id), Ring A is a spiro bicyclic heterocycloalkyl.

In some embodiments of a compound of Formula (I) or (Ia)-(Id), Ring A is

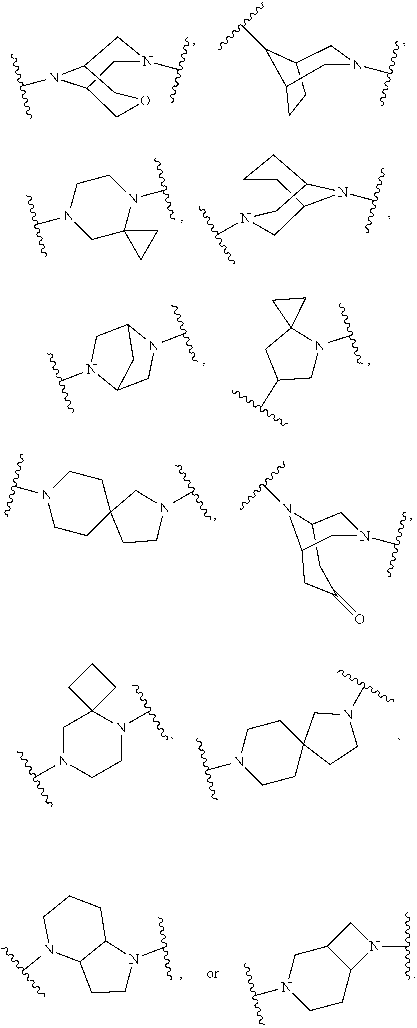

In some embodiments of a compound of Formula (I) or (Ia)-(Id), Ring A is

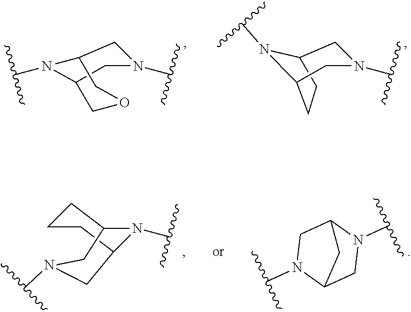 [SMILES: *N1CC2COCC(C1)N2* ; *N1CC2CCC(C1)N2* ; *N1CC2CCCC(C1)N2* ; *N1CC2CC1CN2*]

In some embodiments of a compound of Formula (I) or (Ia)-(Id), Ring A is,

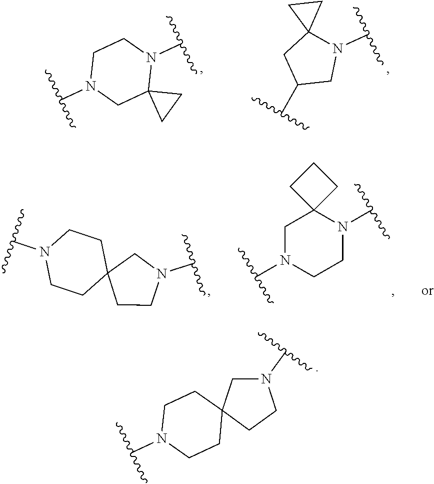 [SMILES: *N1CCN(*)C2(CC2)C1 ; *C1CN(*)C2(CC2)C1 ; *N1CCC2(CC1)CCN(*)C2 ; *N1CCN(*)C2(CCC2)C1 ; *N1CCC2(CC1)CCN(*)C2]

In some embodiments of a compound of Formula (I) or (Ia)-(Id), Ring A is

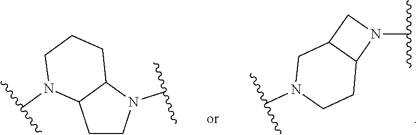 [SMILES: *N1CCCC2C1CCN2* ; *N1CCC2C(C1)CN2*]

In some embodiments of a compound of Formula (I) or (Ia)-(Id), the heterocycloalkyl in Ring A comprises one, two, or three heteroatoms selected from the group consisting of O, N, or S. In some embodiments of a compound of Formula (I) or (Ia)-(Id), the heterocycloalkyl in Ring A comprises one, two, or three heteroatoms selected from the group consisting of O or N. In some embodiments of a compound of Formula (I) or (Ia)-(Id), the heterocycloalkyl in Ring A comprises one, two, or three heteroatoms that are N. In some embodiments of a compound of Formula (I) or (Ia)-(Id), the heterocycloalkyl in Ring A comprises one or two heteroatoms that are N.

In some embodiments of a compound of Formula (I) or (Ia)-(Id), each R⁶ is independently halogen, C₁-C₆alkyl, or C₁-C₆haloalkyl.

In some embodiments of a compound of Formula (I) or (Ia)-(Id), each R⁶ is independently halogen or C₁-C₆alkyl.

In some embodiments of a compound of Formula (I) or (Ia)-(Id), n is 0 or 1. In some embodiments of a compound of Formula (I) or (Ia)-(Id), n is 0-2. In some embodiments of a compound of Formula (I) or (Ia)-(Id), n is 0-3. In some embodiments of a compound of Formula (I) or (Ia)-(Id), n is 0-4. In some embodiments of a compound of Formula (I) or (Ia)-(Id), n is 1 or 2. In some embodiments of a compound of Formula (I) or (Ia)-(Id), n is 1-3. In some embodiments of a compound of Formula (I) or (Ia)-(Id), n is 1-4. In some embodiments of a compound of Formula (I) or (Ia)-(Id), n is 0. In some embodiments of a compound of Formula (I) or (Ia)-(Id), n is 1. In some embodiments of a compound of Formula (I) or (Ia)-(Id), n is 2. In some embodiments of a compound of Formula (I) or (Ia)-(Id), n is 3.

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl. C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R is independently C₁-C₆alkyl, C₁-C₆haloalkyl, C₁-C₆deuteroalkyl, C₁-C₆ hydroxyalkyl, C₁-C₆aminoalkyl, C₁-C₆heteroalkyl, cycloalkyl, or heterocycloalkyl. In some embodiments of a compound disclosed herein, each R¹ is independently C₁-C₆alkyl, C₁-C₆haloalkyl, cycloalkyl, or heterocycloalkyl. In some embodiments of a compound disclosed herein, each R⁴ is independently C₁-C₆alkyl or C₁-C₆haloalkyl. In some embodiments of a compound disclosed herein, each R^{a} is independently C₁-C₆alkyl.

In some embodiments of a compound disclosed herein, each R¹ is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C_{f}alkylene(heterocycloalkyl); wherein each alkyl, alkylene, cycloalkyl, and heterocycloalkyl is independently optionally substituted with one or more R. In some embodiments of a compound disclosed herein, each R¹ is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound disclosed herein, each R^{b} is independently hydrogen, C₁-C₆alkyl, C₁-C₆haloalkyl, cycloalkyl, or heterocycloalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R¹ is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl. C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R¹ and R^{d} are independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R¹ and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound disclosed herein, each R¹ and R^{d} are independently hydrogen, C₁-C₆alkyl. C₁-C₆haloalkyl, cycloalkyl, or heterocycloalkyl. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 3- to 6-membered cycloalkyl, or 3- to 6-membered heterocycloalkyl; or two R on the same atom are taken together to form an oxo. In some embodiments of a compound disclosed herein, each R is independently halogen, —CN, —OH, —NH₂, —NHC₁-C₃alkyl, —N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, or C₁-C₃heteroalkyl; or two R on the same atom are taken together to form an oxo. In some embodiments of a compound disclosed herein, each R is independently halogen, —CN, —OH, —NH₂, —NHC₁-C₃alkyl, —N(C₁-C₃alkyl)₂, C₁-C₃alkyl, C₁-C₃alkoxy, or C₁-C₃haloalkyl; or two R on the same atom are taken together to form an oxo. In some embodiments of a compound disclosed herein, each R is independently halogen, —CN, —OH, —NH₂, C₁-C₃alkyl, C₁-C₃alkoxy, or C₁-C₃haloalkyl; or two R on the same atom are taken together to form an oxo. In some embodiments of a compound disclosed herein, each R is independently halogen, —CN, —OH, —NH₂, C₁-C₃alkyl, or C₁-C₃haloalkyl; or two R on the same atom are taken together to form an oxo. In some embodiments of a compound disclosed herein, each R is independently halogen, C₁-C₃alkyl, or C₁-C₃haloalkyl; or two R on the same atom are taken together to form an oxo.

In some embodiments of a compound disclosed herein, the compound is selected from a compound of Table 1:

TABLE 1

| Ex. | Structure | Name |
|---|---|---|
| 1 | 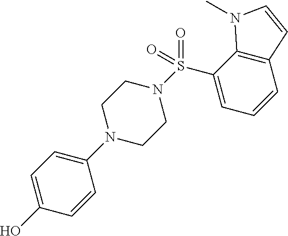 [SMILES: Cn1ccc2cccc(S(=O)(=O)N3CCN(c4ccc(O)cc4)CC3)c21] | 4-(4-((1-methyl-1H-indol-7- yl)sulfonyl)piperazin-1- yl)phenol |
| 2 | 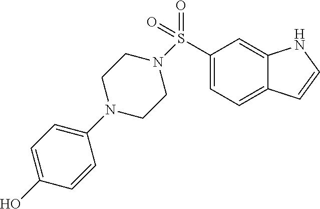 [SMILES: O=S(=O)(c1ccc2cc[nH]c2c1)N1CCN(c2ccc(O)cc2)CC1] | 4-(4-((1H-indol-6- yl)sulfonyl)piperazin-1- yl)phenol |
| 3 | 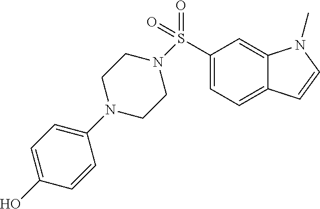 [SMILES: Cn1ccc2ccc(S(=O)(=O)N3CCN(c4ccc(O)cc4)CC3)cc21] | 4-(4-((1-methyl-1H-indol-6- yl)sulfonyl)piperazin-1- yl)phenol |
| 4 | 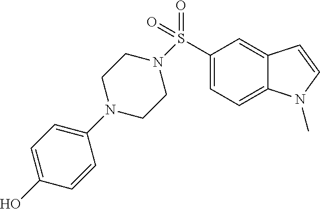 [SMILES: Cn1ccc2cc(S(=O)(=O)N3CCN(c4ccc(O)cc4)CC3)ccc21] | 4-(4-((1-methyl-1H-indol-5- yl)sulfonyl)piperazin-1- yl)phenol |
| 5 | 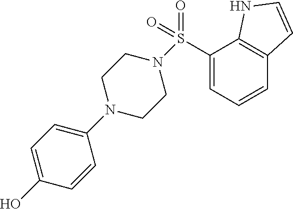 [SMILES: O=S(=O)(c1cccc2cc[nH]c12)N1CCN(c2ccc(O)cc2)CC1] | 4-(4-((1H-indol-7- yl)sulfonyl)piperazin-1- yl)phenol |
| 6 | 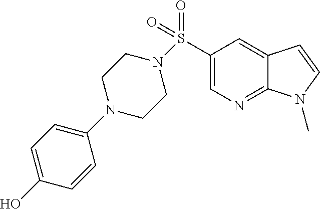 [SMILES: Cn1ccc2cc(S(=O)(=O)N3CCN(c4ccc(O)cc4)CC3)cnc21] | 4-(4-((1-methyl-1H- pyrrolo[2,3-b]pyridin-5- yl)sulfonyl)piperazin-1- yl)phenol |
| 7 | 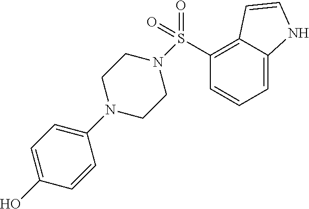 [SMILES: O=S(=O)(c1cccc2[nH]ccc12)N1CCN(c2ccc(O)cc2)CC1] | 4-(4-((1H-indol-4- yl)sulfonyl)piperazin-1- yl)phenol |
| 8 | 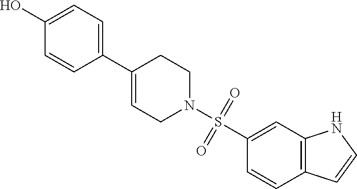 [SMILES: O=S(=O)(c1ccc2cc[nH]c2c1)N1CC=C(c2ccc(O)cc2)CC1] | 4-[1-(1H-indol-6-ylsulfonyl)- 3,6-dihydro-2H-pyridin-4- yl]phenol |
| 9 | 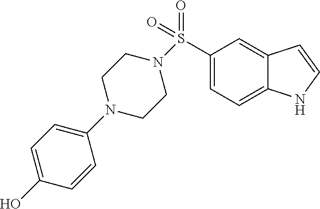 [SMILES: O=S(=O)(c1ccc2[nH]ccc2c1)N1CCN(c2ccc(O)cc2)CC1] | 4-(4-((1H-indol-5- yl)sulfonyl)piperazin-1- yl)phenol |
| 10 | 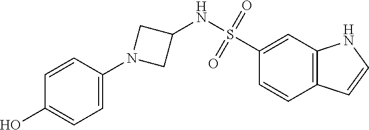 [SMILES: O=S(=O)(NC1CN(c2ccc(O)cc2)C1)c1ccc2cc[nH]c2c1] | N-(1-(4- hydroxyphenyl)azetidin-3- yl)-1H-indole-6-sulfonamide |
| 11 | 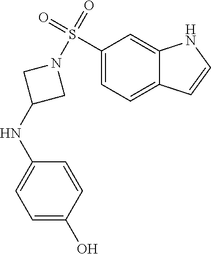 [SMILES: O=S(=O)(c1ccc2cc[nH]c2c1)N1CC(Nc2ccc(O)cc2)C1] | 4-((1-((1H-indol-6- yl)sulfonyl)azetidin-3- yl)amino)phenol |
| 12 | 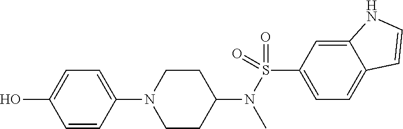 [SMILES: CN(C1CCN(c2ccc(O)cc2)CC1)S(=O)(=O)c1ccc2cc[nH]c2c1] | N-(1-(4- hydroxyphenyl)piperidin-4- yl)-N-methyl-1H-indole-6- sulfonamide |
| 13 | 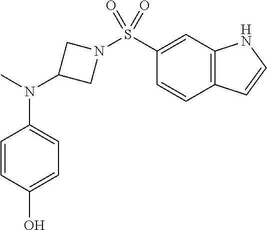 [SMILES: CN(c1ccc(O)cc1)C1CN(S(=O)(=O)c2ccc3cc[nH]c3c2)C1] | 4-((1-((1H-indol-6- yl)sulfonyl)azetidin-3- yl)(methyl)amino)phenol |
| 14 | 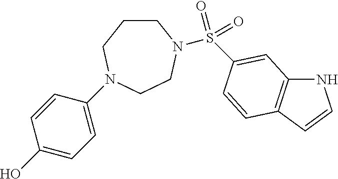 [SMILES: O=S(=O)(c1ccc2cc[nH]c2c1)N1CCCN(c2ccc(O)cc2)CC1] | 4-(4-((1H-indol-6- yl)sulfonyl)-1,4-diazepan-1- yl)phenol |
| 15 | 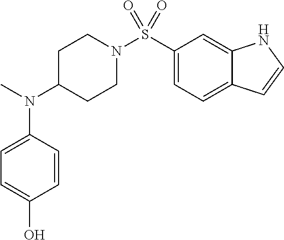 [SMILES: CN(c1ccc(O)cc1)C1CCN(S(=O)(=O)c2ccc3cc[nH]c3c2)CC1] | 4-((1-((1H-indol-6- yl)sulfonyl)piperidin-4- yl)(methyl)amino)phenol |
| 16 | 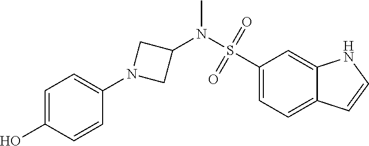 [SMILES: CN(C1CN(c2ccc(O)cc2)C1)S(=O)(=O)c1ccc2cc[nH]c2c1] | N-(1-(4- hydroxyphenyl)azetidin-3- yl)-N-methyl-1H-indole-6- sulfonamide |
| 17 | 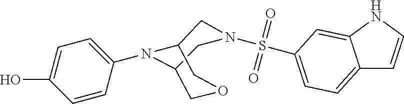 [SMILES: O=S(=O)(c1ccc2cc[nH]c2c1)N1CC2COCC(C1)N2c1ccc(O)cc1] | 4-((1R,5S)-7-((1H-indol-6- yl)sulfonyl)-3-oxa-7,9- diazabicyclo[3.3.1]nonan-9- yl)phenol |
| 18 | 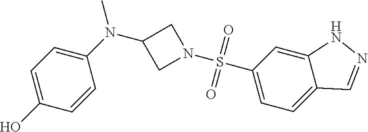 [SMILES: CN(c1ccc(O)cc1)C1CN(S(=O)(=O)c2ccc3cn[nH]c3c2)C1] | 4-((1-((1H-indazol-6- yl)sulfonyl)azetidin-3- yl)(methyl)amino)phenol |
| 19 | 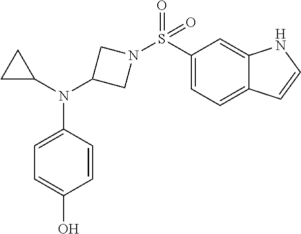 [SMILES: O=S(=O)(c1ccc2cc[nH]c2c1)N1CC(N(c2ccc(O)cc2)C2CC2)C1] | 4-((1-((1H-indol-6- yl)sulfonyl)azetidin-3- yl)(cyclopropyl)amino)phenol |
| 20 | 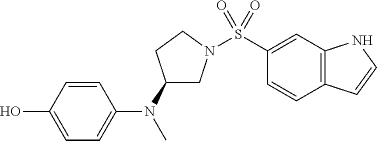 [SMILES: CN(c1ccc(O)cc1)[C@H]1CCN(S(=O)(=O)c2ccc3cc[nH]c3c2)C1] | rac-(R)-4-((1-((1H-indol-6- yl)sulfonyl)pyrrolidin-3- yl)(methyl)amino)phenol |
| 21 | 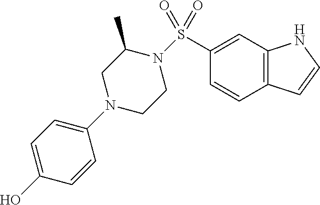 [SMILES: C[C@@H]1CN(c2ccc(O)cc2)CCN1S(=O)(=O)c1ccc2cc[nH]c2c1] | (R)-4-(4-((1H-indol-6- yl)sulfonyl)-3- methylpiperazin-1-yl)phenol |
| 22 | 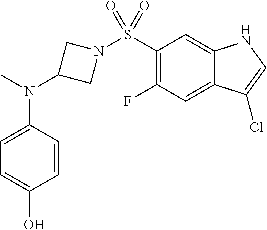 [SMILES: CN(c1ccc(O)cc1)C1CN(S(=O)(=O)c2cc3[nH]cc(Cl)c3cc2F)C1] | 4-((1-((3-chloro-5-fluoro-1H- indol-6-yl)sulfonyl)azetidin- 3-yl)(methyl)amino)phenol |
| 23 | 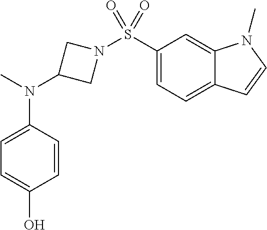 [SMILES: CN(c1ccc(O)cc1)C1CN(S(=O)(=O)c2ccc3ccn(C)c3c2)C1] | 4-(methyl(1-((1-methyl-1H- indol-6-yl)sulfonyl)azetidin- 3-yl)amino)phenol |
| 24 | 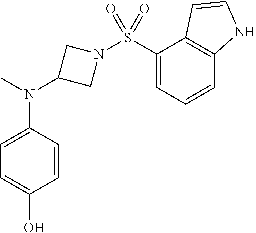 [SMILES: CN(c1ccc(O)cc1)C1CN(S(=O)(=O)c2cccc3[nH]ccc23)C1] | 4-((1-((1H-indol-4- yl)sulfonyl)azetidin-3- yl)(methyl)amino)phenol |
| 25 | 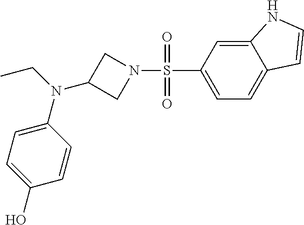 [SMILES: CCN(c1ccc(O)cc1)C1CN(S(=O)(=O)c2ccc3cc[nH]c3c2)C1] | 4-((1-((1H-indol-6- yl)sulfonyl)azetidin-3- yl)(ethyl)amino)phenol |
| 26 | 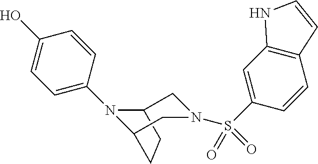 [SMILES: O=S(=O)(c1ccc2cc[nH]c2c1)N1CC2CCC(C1)N2c1ccc(O)cc1] | 4-((1R,5S)-3-((1H-indol-6- yl)sulfonyl)-3,8- diazabicyclo[3.2.1]octan-8- yl)phenol |
| 27 | 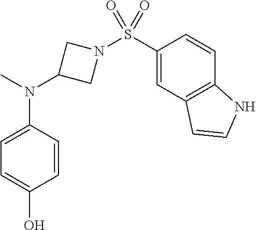 [SMILES: CN(c1ccc(O)cc1)C1CN(S(=O)(=O)c2ccc3[nH]ccc3c2)C1] | 4-((1-((1H-indol-5- yl)sulfonyl)azetidin-3- yl)(methyl)amino)phenol |
| 28 | 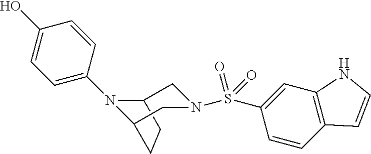 [SMILES: O=S(=O)(c1ccc2cc[nH]c2c1)N1CC2CCC(C1)N2c1ccc(O)cc1] | 4-((1R,5S)-3-((1H-indol-6- yl)sulfonyl)-3,9- diazabicyclo[3.3.1]nonan-9- yl)phenol |
| 29 | 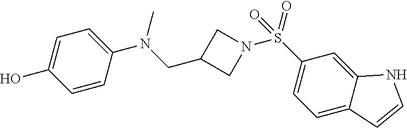 [SMILES: CN(CC1CN(S(=O)(=O)c2ccc3cc[nH]c3c2)C1)c1ccc(O)cc1] | 4-(((1-((1H-indol-6- yl)sulfonyl)azetidin-3- yl)methyl)(methyl)amino) phenol |
| 30 | 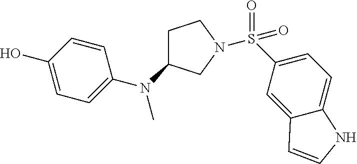 [SMILES: CN(c1ccc(O)cc1)[C@H]1CCN(S(=O)(=O)c2ccc3[nH]ccc3c2)C1] | rac-(R)-4-((1-((1H-indol-5- yl)sulfonyl)pyrrolidin-3- yl)(methyl)amino)phenol |
| 31 | 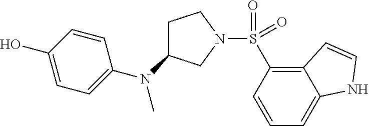 [SMILES: CN(c1ccc(O)cc1)[C@H]1CCN(S(=O)(=O)c2cccc3[nH]ccc23)C1] | rac-(R)-4-((1-((1H-indol-4- yl)sulfonyl)pyrrolidin-3- yl)(methyl)amino)phenol |
| 32 | 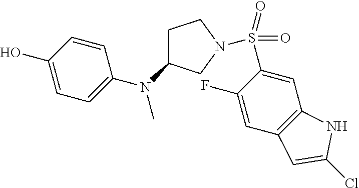 [SMILES: CN(c1ccc(O)cc1)[C@H]1CCN(S(=O)(=O)c2cc3[nH]c(Cl)cc3cc2F)C1] | rac-(R)-4-((1-((2-chloro-5- fluoro-1H-indol-6- yl)sulfonyl)pyrrolidin-3- yl)(methyl)amino)phenol |
| 33 | 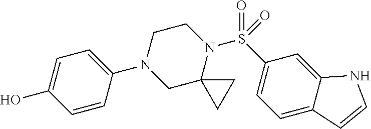 [SMILES: O=S(=O)(c1ccc2cc[nH]c2c1)N1CCN(c2ccc(O)cc2)CC12CC2] | 4-(4-((1H-indol-6- yl)sulfonyl)-4,7- diazaspiro[2.5]octan-7- yl)phenol |
| 34 | 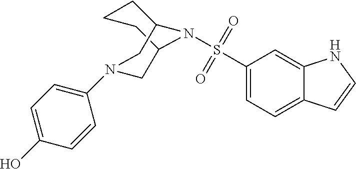 [SMILES: O=S(=O)(c1ccc2cc[nH]c2c1)N1C2CCCC1CN(c1ccc(O)cc1)C2] | 4-((1R,5S)-9-((1H-indol-6- yl)sulfonyl)-3,9- diazabicyclo[3.3.1]nonan-3- yl)phenol |
| 35 | 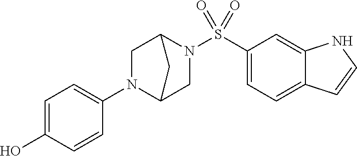 [SMILES: O=S(=O)(c1ccc2cc[nH]c2c1)N1CC2CC1CN2c1ccc(O)cc1] | 4-(5-((1H-indol-6- yl)sulfonyl)-2,5- diazabicyclo[2.2.1]heptan-2- yl)phenol |
| 36 | 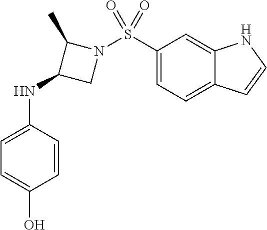 [SMILES: C[C@@H]1[C@H](Nc2ccc(O)cc2)CN1S(=O)(=O)c1ccc2cc[nH]c2c1] | rac-4-(((2R,3R)-1-((1H-indol- 6-yl)sulfonyl)-2- methylazetidin-3- yl)amino)phenol |
| 37 | 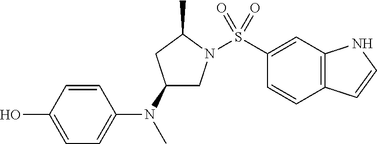 [SMILES: C[C@@H]1C[C@H](N(C)c2ccc(O)cc2)CN1S(=O)(=O)c1ccc2cc[nH]c2c1] | rac-4-(((3R,5S)-1-((1H-indol- 6-yl)sulfonyl)-5- methylpyrrolidin-3- yl)(methyl)amino)phenol |
| 38 | 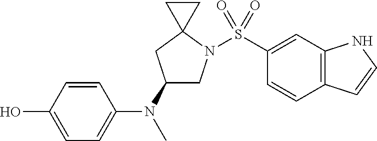 [SMILES: CN(c1ccc(O)cc1)[C@@H]1CN(S(=O)(=O)c2ccc3cc[nH]c3c2)C2(CC2)C1] | rac-(R)-4-((4-((1H-indol-6- yl)sulfonyl)-4- azaspiro[2.4]heptan-6- yl)(methyl)amino)phenol |
| 39 | 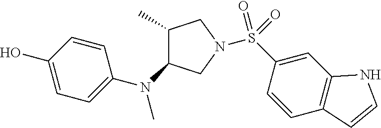 [SMILES: C[C@@H]1CN(S(=O)(=O)c2ccc3cc[nH]c3c2)C[C@H]1N(C)c1ccc(O)cc1] | rac-4-(((3R,4S)-1-((1H-indol- 6-yl)sulfonyl)-4- methylpyrrolidin-3- yl)(methyl)amino)phenol |
| 40 | 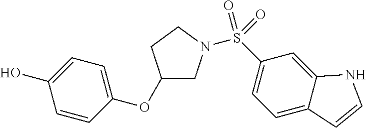 [SMILES: O=S(=O)(c1ccc2cc[nH]c2c1)N1CCC(Oc2ccc(O)cc2)C1] | 4-((1-((1H-indol-6- yl)sulfonyl)pyrrolidin-3- yl)oxy)phenol |
| 41 | 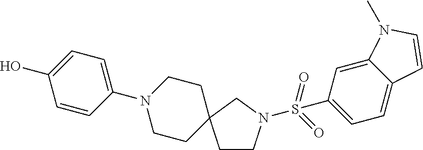 [SMILES: Cn1ccc2ccc(S(=O)(=O)N3CCC4(CCN(c5ccc(O)cc5)CC4)C3)cc21] | 4-(2-((1-methyl-1H-indol-6- yl)sulfonyl)-2,8- diazaspiro[4.5]decan-8- yl)phenol |
| 42 | 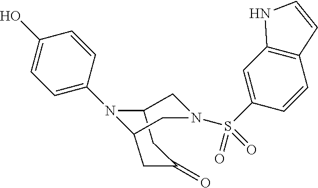 [SMILES: O=C1CC2CN(S(=O)(=O)c3ccc4cc[nH]c4c3)CC(C1)N2c1ccc(O)cc1] | (1R,5S)-3-((1H-indol-6- yl)sulfonyl)-9-(4- hydroxyphenyl)-3,9- diazabicyclo[3.3.1]nonan-7- one |
| 43 | 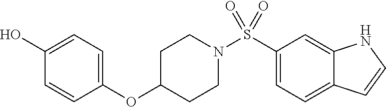 [SMILES: O=S(=O)(c1ccc2cc[nH]c2c1)N1CCC(Oc2ccc(O)cc2)CC1] | 4-((1-((1H-indol-6- yl)sulfonyl)piperidin-4- yl)oxy)phenol |
| 44 | 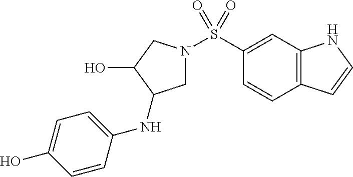 [SMILES: O=S(=O)(c1ccc2cc[nH]c2c1)N1CC(O)C(Nc2ccc(O)cc2)C1] | 1-((1H-indol-6-yl)sulfonyl)- 4-((4- hydroxyphenyl)amino) pyrrolidin-3-ol |
| 45 | 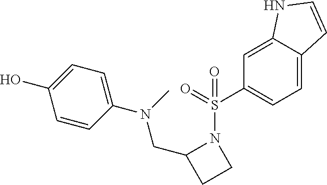 [SMILES: CN(CC1CCN1S(=O)(=O)c1ccc2cc[nH]c2c1)c1ccc(O)cc1] | 4-(((1-((1H-indol-6- yl)sulfonyl)azetidin-2- yl)methyl)(methyl)amino) phenol |
| 46 | 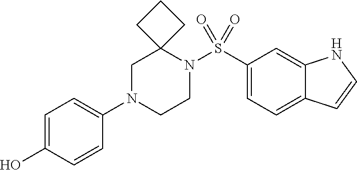 [SMILES: O=S(=O)(c1ccc2cc[nH]c2c1)N1CCN(c2ccc(O)cc2)CC12CCC2] | 4-(5-((1H-indol-6- yl)sulfonyl)-5,8- diazaspiro[3.5]nonan-8- yl)phenol |
| 47 | 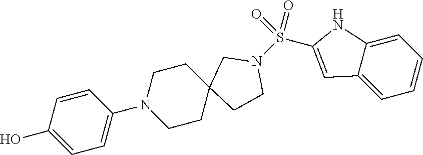 [SMILES: O=S(=O)(c1cc2ccccc2[nH]1)N1CCC2(CCN(c3ccc(O)cc3)CC2)C1] | 4-(2-((1H-indol-2- yl)sulfonyl)-2,8- diazaspiro[4.5]decan-8- yl)phenol |
| 48 | 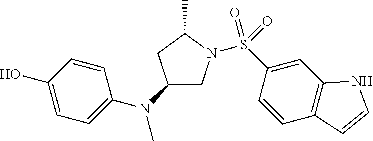 [SMILES: C[C@H]1C[C@H](N(C)c2ccc(O)cc2)CN1S(=O)(=O)c1ccc2cc[nH]c2c1] | rac-4-(((3R,5R)-1-((1H-indol- 6-yl)sulfonyl)-5- methylpyrrolidin-3- yl)(methyl)amino)phenol |
| 49 | 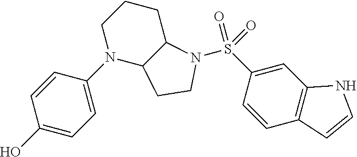 [SMILES: O=S(=O)(c1ccc2cc[nH]c2c1)N1CCC2C1CCCN2c1ccc(O)cc1] | 4-(1-((1H-indol-6- yl)sulfonyl)octahydro-4H- pyrrolo[3,2-b]pyridin-4- yl)phenol |
| 50 | 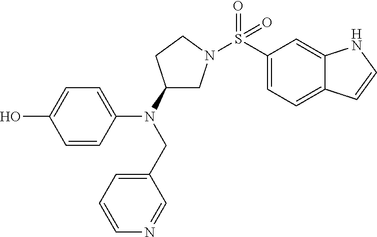 [SMILES: O=S(=O)(c1ccc2cc[nH]c2c1)N1CC[C@H](N(Cc2cccnc2)c2ccc(O)cc2)C1] | (S)-4-((1-((1H-indol-6- yl)sulfonyl)pyrrolidin-3- yl)(pyridin-3- ylmethyl)amino)phenol |
| 51 | 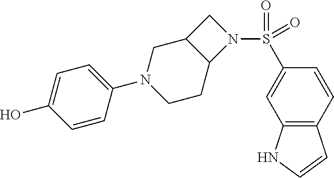 [SMILES: O=S(=O)(c1ccc2cc[nH]c2c1)N1CC2CN(c3ccc(O)cc3)CCC21] | 4-(7-((1H-indol-6- yl)sulfonyl)-3,7- diazabicyclo[4.2.0]octan-3- yl)phenol |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, 7-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like. Representative salts include the alkali or alkaline earth salts, like lithium, sodium, potassium, calcium, and magnesium, and aluminum salts and the like of the tetrazole.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product. Inc. (New Brunswick, NJ), TCI America (Portland, OR), Trans World Chemicals, Inc. (Rockville. MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc. New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”. Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes: “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts,” Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached.

### Methods of Treatment

Disclosed herein are methods for treating cancer in a subject in need thereof, including administering to the subject a therapeutically effective amount of a compound disclosed herein, or a pharmaceutically acceptable salt, solvate, or stereoisomer thereof. Disclosed herein are methods for treating a RNR-related cancer in a subject in need thereof, including administering to the subject a therapeutically effective amount of a compound disclosed herein, or a pharmaceutically acceptable salt, solvate, or stereoisomer thereof.

In some embodiments, the RNR-related cancer includes malignant tumors whose incidence can be decreased or whose symptom is in remission or alleviated and/or completely cured by deleting or suppressing and/or inhibiting functions of RNR. Malignant tumors of interest is, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors, skin cancer, brain tumor and the like.

In some embodiments, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, or stereoisomer thereof, pharmaceutical compositions include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, Herceptin (trastuzumab) resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus, Medulloblastoma, Hodgkin's Disease. Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, lobular carcinoma, ductal carcinoma, cancer of the pancreatic stellate cells, cancer of the hepatic stellate cells, or prostate cancer. In embodiments, the cancer is selected from ovarian cancer, prostate cancer, esophageal cancer, salivary gland cancer, breast cancer, liver cancer, pancreatic cancer, stomach cancer, lung cancer, bladder cancer, colon cancer, and uterine cancer. In embodiments, the cancer is selected from muscle cancer, brain cancer, lymph node cancer, thyroid cancer, kidney cancer, and adrenal gland cancer.

ecDNA mediates an important and clinically distinct mechanism of resistance to targeted therapies. There are immediate therapeutic opportunities for utility of the one or more RNR inhibitor described herein as a single agent or in combination with other therapies. In some embodiments, the one or more RNR inhibitor described herein may be used to treat an ecDNA+cancer, ecDNA+tumor or ecDNA+tumor cells. One or more RNR inhibitor described herein may be used to treat tumors, such as with one or more amplified oncogenes (e.g. FGFR. EGFR, MET, KRAS, MDM2 amplifications), in some cases, the one or more amplified oncogenes comprise non-mutant forms of the oncogene and in some cases, the amplified oncogenes comprises mutant forms of the oncogenes. In some cases, the tumor comprises one or more amplified oncogenes present on ecDNA and the one or more RNR inhibitor described herein are used to treat the tumor in combination with a therapeutic agent targeted to (e.g., an inhibitor of) the one or more amplified oncogenes on the ecDNA. One or more RNR inhibitor described herein may be used to treat tumors for which there are no approved targeted therapies or for which highly efficacious therapies are lacking. One or more RNR inhibitor described herein may be used to treat tumors that have developed resistance to another therapy such as a resistance to a targeted agent. In some cases, a tumor (or tumor cells) treated with one or more targeted agents develops resistance to a targeted agent, such as a targeted agent directed to an oncogene or a targeted agent that directly inhibits activating mutant forms of certain oncoproteins (e.g. KRAS, BRAF, EGFR) or as a consequence of focal amplification such as ecDNA-based amplification of the target gene itself, and the one or more RNR inhibitor described herein may be used to treat such tumors or tumor cells, alone or in combination with an additional therapeutic agent.

Provided herein are methods wherein inhibition of RNR by the one or more RNR inhibitors described herein exhibits synthetic lethality with a cancer-targeted agent. In some embodiments, synthetic lethality arises with one or more RNR inhibitors described herein in combination with a cancer targeted agent. In some cases, a tumor background is identified as hyper-sensitive to a RNR inhibitor and allows a sufficient therapeutic index to enable tolerated doses that are efficacious. In some embodiments, synthetic lethality arises with one or more RNR inhibitors described herein in combination with a cancer targeted agent where the tumor or tumor cells are ecDNA+. In some cases, RNR inhibition results in reduced ecDNA copy number. In some cases, RNR inhibition results in enhanced cytotoxicity in ecDNA+ cells. In some cases, enhanced cytotoxicity results from the combination of RNR inhibition and inhibition of a cancer-target, such as an oncogene, for example an oncogene amplified on ecDNA.

In an aspect of methods herein, a tumor or tumor cells to be treated are ecDNA+. In some cases, such tumor or tumor cells are determined to have an ecDNA signature. In some cases, a tumor or tumor cells are determined to have an ecDNA signature when the tumor or tumor cells have one or more characteristics associated with ecDNA+tumors or tumor cells. For example, in some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof.

### Combination Therapy

In certain instances, the compound described herein, or a pharmaceutically acceptable salt, solvate, or stereoisomer thereof, is administered in combination with a second therapeutic agent or a cancer-targeted agent.

In an aspect of methods herein, the method further comprises administering a cancer-targeted therapeutic agent, directed to an activity of a protein product of a target gene. In some cases, the treatment with the cancer-targeted therapeutic agent and the RNR inhibitor disclosed herein reduces amplification or expression of the target gene in the tumor or tumor cells. In some cases, the cancer-targeted therapeutic agent is administered prior to the RNR inhibitor. In some cases, the cancer-targeted therapeutic agent is administered concurrently with the RNR inhibitor.

In an aspect of methods herein, the tumor or tumor cells have an ecDNA signature. In some cases, the tumor or tumor cells develop the ecDNA signature after administration of the cancer-targeted therapeutic agent. In some cases, the tumor or tumor cells develop the ecDNA signature prior to treatment. In some cases, the method prevents an increase of ecDNA in the tumor or tumor cells.

In some embodiment, the second therapeutic includes antimetabolites, platinum drugs, plant alkaloid drugs, and molecular targeting drugs.

In some embodiments, the antimetabolites include 5-fluorouracil, 5-fluoro-2′-deoxyuridine, tegafur, tegafur-uracil, tegafur-gimeracil-oteracil, pemetrexed, trifluridine, trifluridine-tipiracil hydrochloride, fludarabine (or an active metabolite fludarabine nucleoside), cytarabine, gemcitabine, capecitabine, nelarabine, clofarabine, and DNA methylation inhibitors (decitabine, guadecitabine, azacitidine, etc.).

In some embodiments, the platinum drugs include cisplatin, oxaliplatin, carboplatin, and nedaplatin.

In some embodiments, the plant alkaloid drugs include microtube inhibiting drugs such as paclitaxel, docetaxel, vinblastine, vincristine, vindesine, vinorelbine, and eribulin, and topoisomerase inhibiting drugs such as irinotecan (or an active metabolite SN-38), nogitecan, and etoposide.

In some embodiments, the molecular targeting drugs include ATR (ataxia telangiectasia and Rad3 related protein) inhibitors, Chk1 (checkpoint kinase 1) inhibitors. HSP (heat shock protein) 90 inhibitors, PARP (poly ADP ribose polymerase) inhibitors, EGFR (epidermal growth factor receptor) inhibitors. Her2 inhibitors, VEGFR (vascular endothelial growth factor receptor) inhibitors, PDGFR (platelet-derived growth factor receptor) inhibitors, MET inhibitors, AXL inhibitors, RET inhibitors, FLT3 (fims-related tyrosine kinase 3) inhibitors, KIT inhibitors, CSF1R (colony-stimulating factor 1 receptor) inhibitors, TIE2 (tunica interna endothelial cell kinase 2) inhibitors, TRKB inhibitors, and CDK4/6 inhibitors. In some embodiments, the ATR inhibitors include AZD6738, berzosertib. BAY1895344, and VX-803. In some embodiments, the Chk1 inhibitors include prexasertib, SCH900776, GDC-0575, and CCT245737. In some embodiments, the HSP90 inhibitors include luminespib, ganetespib, and onalespib. In some embodiments, the PARP inhibitors include olaparib, rucaparib, niraparib, veliparib, and talazoparib. In some embodiments, the EGFR inhibitors include small molecule inhibitors such as lapatinib, gefitinib, erlotinib, afatinib, and vandetanib, and anti-EGFR antibodies such as cetuximab and panitumumab. In some embodiments, the Her2 inhibitors include small molecule inhibitors such as lapatinib, and anti-Her2 antibodies such as trastuzumab, pertuzumab, and trastuzumab emtansine. In some embodiments, the VEGFR inhibitors are inhibitors of at least one of VEGFR1, VEGFR2, and VEGFR3 and include small molecule inhibitors such as sunitinib, cabozantinib, midostaurin, sorafenib, vandetanib, pazopanib, lenvatinib, and axitinib, and anti-VEGFR antibodies such as ramucirumab. In some embodiments, the PDGFR inhibitors are PDGFRα and/or PDGFRβ inhibitors and include sunitinib, midostaurin, pazopanib, lenvatinib, and sorafenib. In some embodiments, the MET inhibitors include cabozantinib, crizotinib, and tepotinib. In some embodiments, the AXL inhibitors include cabozantinib and gilteritinib. In some embodiments, the RET inhibitors include sunitinib, cabozantinib, sorafenib, lenvatinib, and vandetanib. In some embodiments, the FLT3 inhibitors include sunitinib, cabozantinib, midostaurin, gilteritinib, and sorafenib. In some embodiments, the KIT inhibitors include sunitinib, midostaurin, pazopanib, lenvatinib, and sorafenib. In some embodiments, the CSF1R inhibitors include sunitinib. BLZ-945, and ARRY-382. In some embodiments, the TIE2 inhibitors include cabozantinib. In some embodiments, the TRKB inhibitors include cabozantinib and entrectinib. In some embodiments, the CDK4/6 inhibitors include palbociclib, ribociclib, and abemaciclib.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, or stereoisomer thereof, is co-administered with a second therapeutic agent, wherein the compound described herein, or a pharmaceutically acceptable salt, solvate, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of the compounds disclosed herein will be utilized in formulating a pharmaceutical composition and/or in treatment regimens when the compounds disclosed herein are administered in combination with a second therapeutic agent. Therapeutically-effective dosages of drugs and other agents for use in combination treatment regimens are optionally determined by means similar to those set forth hereinabove for the actives themselves. Furthermore, the methods of treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, or stereoisomer thereof, and the second agent being used in combination are administered simultaneously, or at different times, and/or at decreasing or increasing intervals during the treatment period. Combination treatment further includes periodic treatments that start and stop at various times to assist with the clinical management of the patient.

It is understood that the dosage regimen to treat or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, or stereoisomer thereof, as well as combination therapies, are administered before, during, or after the occurrence of a disease or condition, and the timing of administering the composition containing a compound varies. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound of described herein, or a pharmaceutically acceptable salt, solvate, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

### EXAMPLES

### Experimental Part

### Method A

### Synthesis of 4-(4-((1-methyl-1H-indol-7-yl)sulfonyl)piperazin-1-yl)phenol (Ex 1)

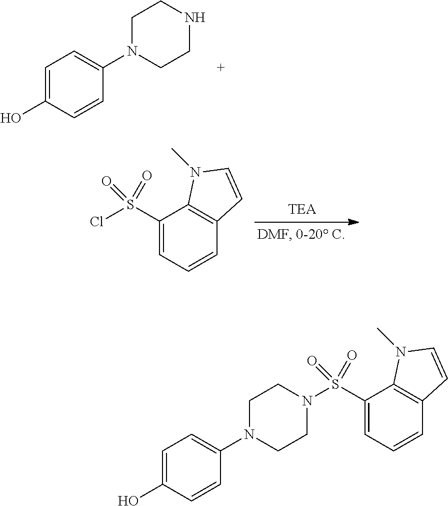 [SMILES: Oc1ccc(N2CCNCC2)cc1 ; Cn1ccc2cccc(S(=O)(=O)Cl)c21 ; Cn1ccc2cccc(S(=O)(=O)N3CCN(c4ccc(O)cc4)CC3)c21]

A solution of 4-(Piperazin-1-yl) phenol (1.00 eq, 40 mg, 0.224 mmol) in DMF (0.8 mL) was added triethylamine (2.50 eq, 0.078 mL, 0.56 mmol) and stirred for 5 minutes at 0° C. Then 1-methyl-1H-indole-7-sulfonyl chloride (1.00 eq, 51.5 mg, 0.224 mmol) in DMF (0.8 ml) was added dropwise at 0°. The reaction mixture was stirred at 0° C. for 30 minutes. The reaction mixture was poured in water (4 ml) and extracted 2 times with ethyl acetate (4 ml). The combined organic phases were washed twice with water (2 ml). The organic layer was evaporated, and residue was purified by a silica gel chromatography 10% methanol in DCM as eluent. The product was isolated (86 mg, 83%). LC-MS: m/z=372 [M+H]⁺. 1H NMR (499 MHz, DMSO) δ 8.90 (s, 1H), 7.95-7.92 (m, 1H), 7.70 (d, J=7.5 Hz, 1H), 7.45 (2, J=2.8 Hz, 1H), 7.19 (t, J=7.7 Hz, 1H), 6.80 (s, 2H), 6.67-6.64 (d, J=8.3 Hz, 2H), 6.66 (s, 1H), 4.15 (s, 3H), 3.32-3.35 (m, 4H), 3.02-3.04 (m, 4H), 4.00-3.49 (m, 17H), 3.24 (s, 9H).

Method A* Boc Deprotection with Acid:

### Synthesis of 4-(4-((1H-indol-7-yl)sulfonyl)piperazin-1-yl)phenol (Ex 5)

The title compound was synthesized in a similar manner to 4-(4-((1-methyl-1H-indol-7-yl)sulfonyl)piperazin-1-yl)phenol (Ex 1) for the first step using tert-butyl 7-chlorosulfonylindole-1-carboxylate, and was isolated as a solid (140 mg, 91%) after purification by flash chromatography with ethyl acetate:hexanes. LC-MS: m/z=458.06 [M+H]⁺. Then the Boc-removal was performed as the following procedure.

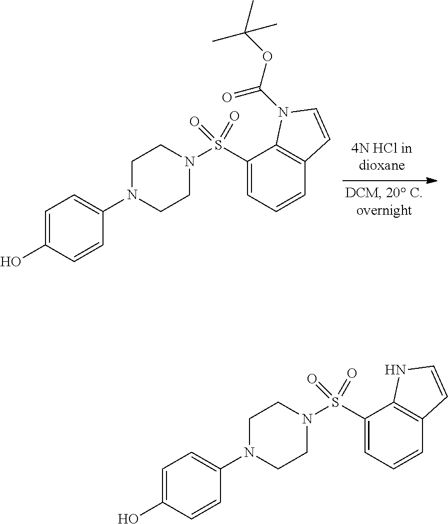 [SMILES: CC(C)(C)OC(=O)n1ccc2cccc(S(=O)(=O)N3CCN(c4ccc(O)cc4)CC3)c21 ; O=S(=O)(c1cccc2cc[nH]c12)N1CCN(c2ccc(O)cc2)CC1]

To solution of tert-butyl 7-[4-(4-hydroxyphenyl)piperazin-1-yl]sulfonylindole-1-carboxylate (1.00 eq, 70 mg, 0.153 mmol) in DCM (4 mL) was added 4N HCl (0.4 ml, 1.53 mmol) in dioxanes. The reaction mixture was stirred at 20° C. overnight. Solids was fallen out solution and was filtered, followed by rinsing with DCM (5 ml). The product was dried under high vacuum to isolate solids (38 mg, 70%). LC-MS: m/z=394 [M+H]⁺. 1H NMR (499 MHz, DMSO) δ 11.19 (s, 1H), 7.97 (d, J=7.8 Hz, 1H), 7.50 (d, J=7.5 Hz, 1H), 7.45 (t, J=2.8 Hz, 1H), 7.25 (t, J=7.7 Hz, 11H), 7.03 (s, 2H), 6.70 (d, J=8.3 Hz, 2H), 6.66 (dd, J=3.0, 1.9 Hz, 1H), 4.00-3.49 (m, 17H), 3.24 (s, 9H).

### Method B

### Synthesis of 4-(4-((1H-indol-6-yl) sulfonyl) piperazin-1-yl) phenol (Ex 2)

### Step 1: 4-(4-((1-(triisopropylsilyl)-1H-indol-6-yl) sulfonyl) piperazin-1-yl) phenol

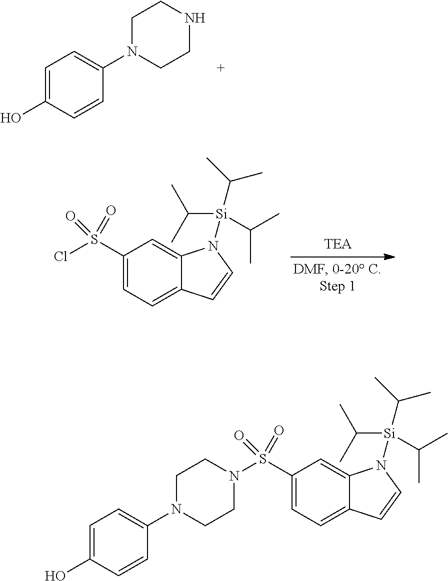 [SMILES: Oc1ccc(N2CCNCC2)cc1 ; CC(C)[Si](C(C)C)(C(C)C)n1ccc2ccc(S(=O)(=O)Cl)cc21 ; CC(C)[Si](C(C)C)(C(C)C)n1ccc2ccc(S(=O)(=O)N3CCN(c4ccc(O)cc4)CC3)cc21]

A solution of 4-(Piperazin-1-yl) phenol (1.00 eq. 40 mg, 0.224 mmol) in DMF (2 mL) was added triethylamine (2.50 eq, 0.078 mL, 0.561 mmol) and stirred for 5 minutes at 0° C. Then 1-triisopropylsilylindole-6-sulfonyl chloride (1.00 eq, 83 mg, 0.224 mmol) was added at 0° C. in portions and continued stirring the reaction mixture at 0° C. for 30 minutes. The reaction mixture was worked up with water (4 ml) and ethyl acetate (4 ml) and washed twice with water (2 ml). The organic layer was evaporated, and residue was purified by a silica gel chromatography with a gradient from 0-100% ethyl acetate in hexane. The product was isolated as a white solid (100 mg, 87%). LC-MS: m/z=513 [M+H]⁺.

### Step 2: 4-(4-((1H-indol-6-yl) sulfonyl) piperazin-1-yl) phenol

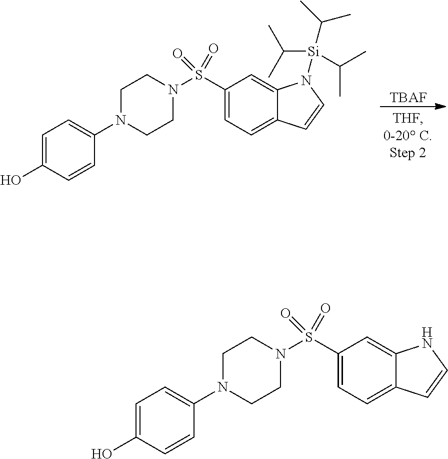

A solution of 4-[4-(1-triisopropylsilanol-6-yl) sulfon ylpiperazin-1-yl] phenol (1.00) eq 100 mg, 0.195 mmol) in THF (4 mL) was added 0.195 ml 2N TBAF in THE (2.00 eq, 0.389 mmol) at 0° C. The reaction mixture continued stirring at 0° C. for 30 minutes and warmed the reaction to 20° C. and stirred for another one hour. The reaction mixture was worked up with water (4 ml) and ethyl acetate (4 ml). The organic layer was dried and purified by silica gel chromatography with a gradient 0-100% ethyl acetate in hexanes. The product was isolated (47 mg+68% yield). LC-MS: m/z=358 [M−H]⁺. 1H NMR (499 MHz, DMSO) δ 11.64 (s, 1H), 8.87 (s, 1H), 7.84 (s, 1H), 7.79 (d, J=8.4 Hz, 1H), 7.71-7.66 (m, 1H, 7.35 (dd, J=8.3, 1.6 Hz, 1H), 6.76-6.70 (m, 2H), 6.61 (d, J=8.9 Hz, 3H), 3.05-2.83 (m, 9H)

The following examples were prepared using the same procedures:

| | | LC/MS | |
|---|---|---|---|
| Ex. | Name | (ES, m/z) | ¹H-NMR |
| 1 | 4-(4-((1-methyl-1H-indol- | m/z = 372 | 1H NMR (499 MHz, DMSO) δ 8.90 (s, 1H), |
| | 7-yl)sulfonyl)piperazin- | [M + H]+ | 7.95-7.92 (m, 1H), 7.70 (d, J = 7.5 Hz, 1H), |
| | 1-yl)phenol | | 7.45 (2, J = 2.8 Hz, 1H), 7.19 (t, J = 7.7 Hz, |
| | | | 1H), 6.80 (s, 2H), 6.67-6.64 (d, J = 8.3 Hz, |
| | | | 2H), 6.66 (s, 1H), 4.15 (s, 3H), 3.32-3.35 (m, |
| | | | 4H), 3.02-3.04 (m, 4H), 4.00-3.49 (m, 17H), |
| | | | 3.24 (s, 9H). |
| 2** | 4-(4-((1H-indol-6- | m/z = | 1H NMR (499 MHz, DMSO) δ 11.64 (s, 1H), |
| | yl)sulfonyl)piperazin- | 358[M + H]+ | 8.87 (s, 1H), 7.84 (s, 1H), 7.79 (d, J = 8.4 Hz, |
| | 1-yl)phenol | | 1H), 7.71-7.66 (m, 1H), 7.35 (dd, J = 8.3, |
| | | | 1.6 Hz, 1H), |
| | | | 6.76-6.70 (m, 2H), 6.61 (d, J = 8.9 Hz, 3H), |
| | | | 3.05-2.83 (m, 9H) |
| 3 | 4-(4-((1-methyl-1H-indol- | m/z = 372 | 1H NMR (499 MHz, DMSO) δ 8.87 (s, 1H), |
| | 6-yl)sulfonyl)piperazin- | [M + H]+ | 7.87 (s, 1H), 7.79 (d, J = 8.4 Hz, 1H), 7.65 (d, |
| | 1-yl)phenol | | J = 3.1 Hz, 1H), 7.39 (dd, J = 8.3, 1.5 Hz, |
| | | | 1H), 6.79-6.69 (m, 2H), 6.61 (dd, J = 6.2, |
| | | | 2.6 Hz, 3H), 3.91 (s, 3H), 3.00 (s, 8H) |
| 4 | 4-(4-((1-methyl-1H-indol- | m/z = 372 | 1H NMR (499 MHz, DMSO) δ 8.87 (s, 1H), |
| | 5-yl)sulfonyl)piperazin- | [M + H]+ | 8.03 (d, J = 1.6 Hz, 1H), 7.69 (d, J = 8,7 Hz, |
| | 1-yl)phenol | | 1H), 7.56 (d, J = 3.1 Hz, 1H), 7.51 (dd, J = |
| | | | 8.7, 1.7 Hz, 1H), 6.74-6.66 (m, 3H), 6.66-6.52 |
| | | | (m, 2H), 3.87 (s, 3H), 2.98 (dd, J = 15.8, |
| | | | 5.2 Hz, 9H) |
| 5* | 4-(4-((1H-indol-7- | m/z = 394 | 1H NMR (499 MHz, DMSO) δ 11.19 (s, 1H), |
| | yl)sulfonyl)piperazin- | [M + H]+ | 7.97 (d, J = 7.8 Hz, 1H), 7.50 (d, J = 7.5 Hz, |
| | 1-yl)phenol | | 1H), 7.45 (t, J = 2.8 Hz, 1H), 7.25 (t, J = 7.7 |
| | | | Hz, 1H), 7.03 (s, 2H), 6.70 (d, J = 8.3 Hz, |
| | | | 2H), 6.66 (dd, J = 3.0, 1.9 Hz, 1H), 4.00-3.49 |
| | | | (m, 17H), 3.24 (s, 9H). |
| 6 | 4-(4-((1-methyl-1H- | m/z = | 1H NMR (499 MHz, DMSO) δ 8.88 (s, 1H), |
| | pyrrolo[2,3-b]pyridin- | 373[M + H]+ | 8.61 (d, J = 2.2 Hz, 1H), 8.41 (d, J = 2.2 Hz, |
| | 5-yl)sulfonyl)piperazin- | | 1H), 7.77 (d, J = 3.5 Hz, 1H), 6.78-6.69 (m, |
| | 1-yl)phenol | | 3H), 6.69-6.56 (m, 2H), 3.90 (s, 3H), 3.31 |
| | | | (s, 7H), 3.01 (s, 9H) |
| 7** | 4-(4-((1H-indol-4- | m/z = | 1H NMR (499 MHz, DMSO) δ 11.69 (s, 1H), |
| | yl)sulfonyl)piperazin- | 358[M + H]+ | 8.85 (s, 1H), 7.76 (d, J = 7.9 Hz, 1H), 7.59 (d, |
| | 1-yl)phenol | | J = 2.9 Hz, 1H), 7.46 (d, J = 7.7 Hz, 1H), 7.30 |
| | | | (t, J = 7.8 Hz, 1H), 6.83 (d, J = 3.0 Hz, 1H). |
| | | | 6.74-6.67 (m, 2H), 6.63-6.55 (m, 2H), 3.06-3.00 |
| | | | (m, 4H), 2.99-2.85 (m, 4H). |
| 8** | 4-[1-(1H-indol-6- | m/z = | 1H NMR (499 MHz, DMSO) δ 11.62 (s, 1H), |
| | ylsulfonyl)-3,6- | 355[M + H]+ | 9.41 (s, 1H), 7.87 (s, 1H), 7.76 (d, J = 8.4 Hz, |
| | dihydro-2H-pyridin- | | 1H), 7.66 (t, J = 2.7 Hz, 1H), 7.38 (dd, J = 8.3, |
| | 4-yl]phenol | | 1.6 Hz, 1H), 7.21-7.10 (m, 2H), 6,73-6.65 |
| | | | (m, 2H), 6.60 (t, J = 2.1 Hz, 1H), 5.90 (t, J = |
| | | | 3.5 Hz, 1H), 3.60 (d, J = 3.0 Hz, 2H), 3.16 (t, |
| | | | J = 5.7 Hz, 2H), 1.99 (s, 1H), 1.17 (t, J = 7.1 |
| | | | Hz, 1H) |
| 9** | 4-(4-((1H-indol-5- | m/z = | 1H NMR (499 MHz, DMSO) δ 11.67 (s, 1H), |
| | yl)sulfonyl)piperazin- | 358[M + H]+ | 8.86 (s, 1H), 8.03 (d, J = 1.6 Hz, 1H), 7.62 (d, |
| | 1-yl)phenol | | J = 8.5 Hz, 1H), 7.58 (s, 1H), 7.46 (dd, J = |
| | | | 8.6, 1.8 Hz, 1H), 6.77-6.70 (m, 2H), 6.68 (d, |
| | | | J = 3.0 Hz, 1H), 6.64-6.58 (m, 2H), 4.03 (q, |
| | | | J = 7.1 Hz, 1H), 3.04-2.90 (m, 8H), 1.99 (s, |
| | | | 1H), 1.17 (t, J = 7.1 Hz, 1H) |

*Boc deprotection with acid (method A*)
**Si deprotection (method B)

### Method C

### Synthesis of 3-((1H-indol-6-yl) sulfonyl)-9-(4-hydroxyphenyl)-3,9-diazabicyclo[3,3,1] nonan-7-one (Ex 17)

### Step 1: tert-butyl 9-(4-(tert-butoxy) phenyl)-7-oxo-3,9-diazabicyclo 133.11 nonane-3-carboxylate

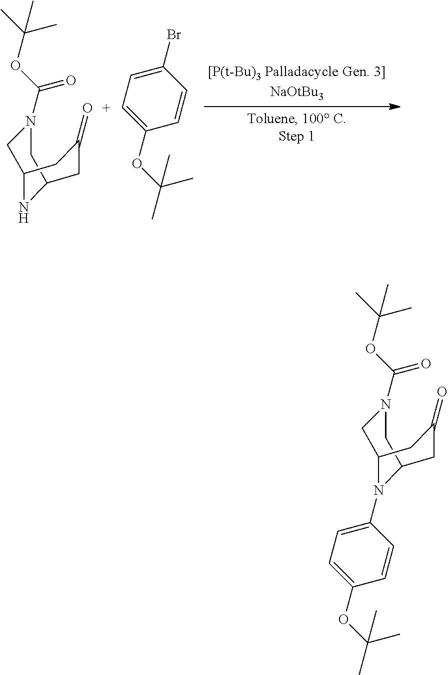

A combination of tert-butyl 7-oxo-3,9-diazabicyclo[3.3.1]nonane-3-carboxylate (1.00 eq, 1000 mg, 4.16 mmol), 1-bromo-4-tert-butoxy-benzene (1.05 eq. 1001 mgs, 4.37 mmol), sodium t-butanolate (2.00 eq, 800 mg, 8.32 mmol), methanesulfonato(tri-t-butylphosphino)(2′-amino-1,1′-biphenyl-2-yl)palladium(II), 98% [P(t-Bu)₃ Palladacycle Gen. 3] (0.0500 eq. 0.14 mL, 0.208 mmol) in seal tube was degassed under argon three times. Toluene (10 mL) was added and heated reaction at 100° C. overnight under argon balloon. The reaction mixture was worked up with water and ethyl acetate. Then the reaction mixture was filtered through a celite short column and solids was filtered. The organic layer was concentrated and purified through a silica gel column with 0-100% ethyl acetate in hexane to isolate product (660 mg, 41%).

### Step 2 & 3: 9-(4-hydroxyphenyl)-3-((1-(triisopropylsilyl)-1H-indol-6-yl) sulfonyl)-3,9-diazabicyclo [3.3.1] nonan-7-one

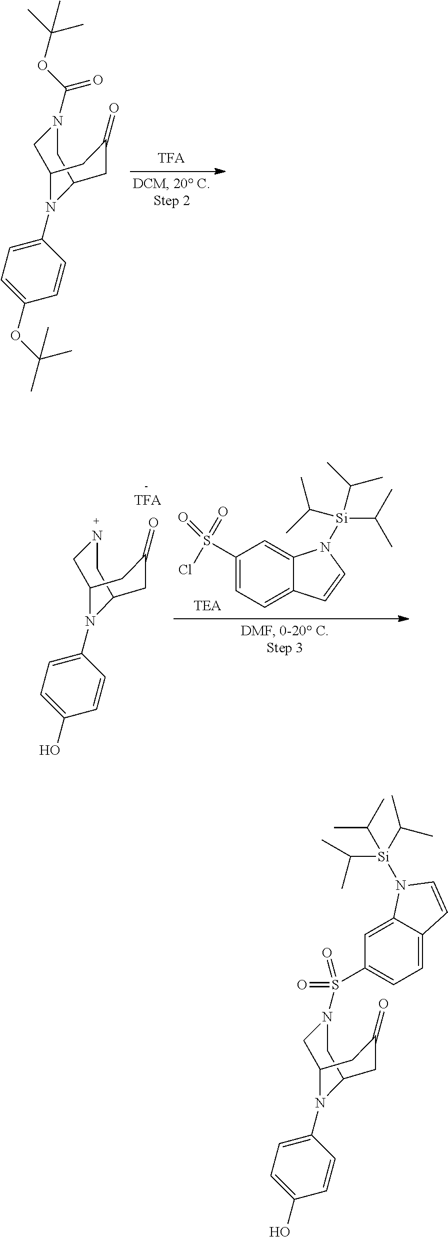

To a solution of tert-butyl 9-(4-hydroxyphenyl)-7-oxo-3,9-diazabicyclo [3.3.1] nonane-3-carboxylate (1.00 eq, 450 mg, 1.35 mmol) in DCM (5 mL) was added TFA (24.3 eq, 1.0 mL, 32.9 mmol). The reaction was stirred 2 hrs at 20° C. The reaction mixture was evaporated and dried under high vacuum and crude product was used directly in the next step. LC-MS: m/z=233 [M+H]⁺. To a solution of 9-(4-hydroxyphenyl)-3,9-diazabicyclo [3.3.1] nonan-7-one (1.00 eq, 314 mg, 1.35 mmol) in DMF (5 mL) was added triethylamine (5.00 eq, 0.94 mL, 6.75 mmol) at 0° C. and followed by addition of 1-[tris(propan-2-yl) silyl]-H-indole-6-sulfonyl chloride (1.00 eq, 502 mg, 1.35 mmol) in portions. The reaction mixture was continued stirring for 20 minutes at 0° C. to completion. Then the reaction was quenched with water and extracted with ethyl acetate. The organic layer was washed twice with 2 ml of water, dried and concentrated to dryness. The product was purified by silica gel column with 0-100% ethyl acetate in hexanes to isolate product (314 mg, 41%). LC-MS: m/z=568[M+H]⁺.

### Step 4: 3-((1H-indol-6-yl) sulfonyl)-9-(4-hydroxyphenyl)-3,9-diazabicyclo 13.3.11 nonan-7-one

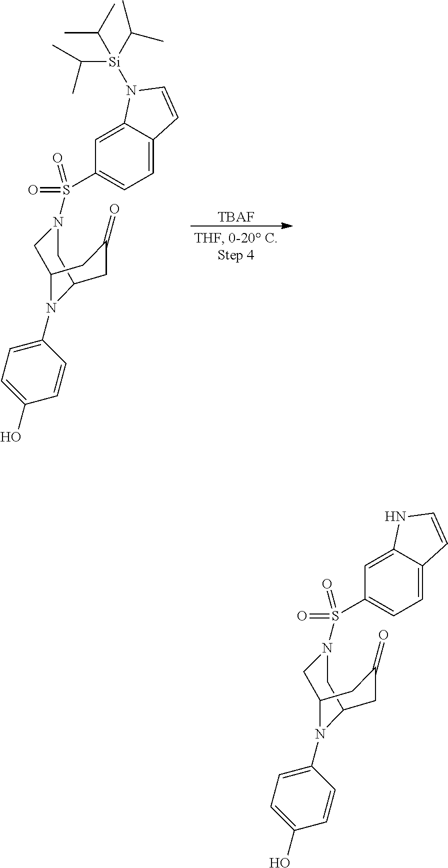

To a solution of 9-(4-hydroxyphenyl)-3-(1-triisopropylsilylindol-6-yl) sulfonyl-3,9-diazabicyclo [3.3.1] nonan-7-one (1.00 eq, 224 mg, 0.394 mmol) in THF (5 mL) was added 0.4 ml of 2N TBAF (2.00 eq, 29 mg, 0.789 mmol) at 0° C. The reaction mixture was brought to 20° C. and stirred for 2 hours. Then the reaction mixture was worked up with water (4 ml) and ethyl acetate (4 ml). The organic layer was concentrated and purified by a silica gel chromatography with a gradient 0-100% ethyl acetate in hexanes. The product was isolated (140 mg/86%). LC-MS: m/z=412 [M+H]⁺. 1H NMR (499 MHz, DMSO) δ 11.66 (s, 1H), 8.79 (s, 11), 7.84-7.70 (m, 2H), 7.67 (t, J=2.8 Hz, 1H), 7.28 (dd, J=8.4, 1.7 Hz, 11), 6.86-6.74 (m, 2H), 6.68-6.55 (m, 3H), 4.33 (d, J=4.5 Hz, 2H), 3.57 (d J=10.7 Hz, 2H), 2.43 (d, J=9.4 Hz, 2H), 2.25 (d, J=15.8 Hz, 2H).

The following examples were prepared using the same procedure:

| | | LC/MS | |
|---|---|---|---|
| Ex. | Name | (ES, m/z) | ¹H-NMR |
| 14 | 4-(4-((1H-indol-6- | m/z = 372.4 | ]+. 1H NMR (400 MHz, DMSO) δ 11.55 (s, 1H), 8.48 (s, |
| | yl)sulfonyl)-1,4- | [M + H]+ | 1H), 7.82 (dd, J = 1.7, 0.9 Hz, 1H), 7.70 (d, J = 8.4 Hz, |
| | diazepan-1-yl)phenol | | 1H), 7.63 (t, J = 2.6 Hz, 1H), 7.32 (dd, J = 8.4, 1.7 Hz, |
| | | | 1H), 6.67-6.21 (m, 5H), 3.49 (t, J = 5.2 Hz, 2H), 3.40 (t, |
| | | | J = 6.1 Hz, 2H), 3.29 (t, J = 5.2 Hz, 2H), 3.06 (dd, J = |
| | | | 6.7, 5.0 Hz, 2H), 1.87 (p, J = 6.0 Hz, 2H) |
| 17 | 4-((1R,5S)-7-((1H-indol- | m/z 400.1 | ]+. 1H NMR (400 MHz, DMSO) δ 11.58 (s, 1H), 8.63 (s, |
| | 6-yl)sulfonyl)-3-oxa-7,9- | [M + H]+ | 1H), 7.76 (dt, J = 1.6, 0.7 Hz, 1H), 7.71 (d, J = 8.4 Hz, |
| | diazabicyclo[3.3.1]nonan- | | 1H), 7.63 (dd, J = 3.2, 1.7 Hz, 1H), 7.27 (dd, J = 8.4, 1.6 |
| | 9-yl)phenol | | Hz, 1H), 6.65-6.45 (m, 5H), 3.91-3.73 (m, 6H), 3.59 |
| | | | (d, J = 11.2 Hz, 2H), 2.78-2.64 (m, 2H) |
| 21 | (R)-4-(4-((1H-indol- | m/z = 372 | 1H NMR (499 MHz, DMSO) δ 11.58 (s, 1H), 8.86 (s, |
| | 6-yl)sulfonyl)-3- | [M + H]+ | 1H), 7.89 (s, 1H), 7.74 (d, J = 8.4 Hz, 1H), 7.68-7.57 |
| | methylpiperazin-1- | | (m, 1H), 7.41 (dd, J = 8.4, 1.7 Hz, 1H), 6.79-6.64 (m, |
| | yl)phenol | | 2H), 6.64-6.50 (m, 3H), 4.12-3.96 (m, 1H), 3.67-3.58 |
| | | | (m, 1H), 3.23 (t, J = 10.7 Hz, 2H), 3.11 (d, J = 11.6 |
| | | | Hz, 1H), 2.55 (dd, J = 11.7, 3.4 Hz, 1H), 2.47-2.38 (m, |
| | | | 1H), 1.09 (d, J = 6.7 Hz, 3H). |
| 22 | 4-((1-((3-chloro-5- | m/z = | 1H NMR (499 MHz, DMSO) δ 11.98 (s, 1H), 8.91 (s, |
| | fluoro-1H-indol-6- | 410[M + H]+ | 1H), 7.94 (d, J = 2.8 Hz, 1H), 7.89 (d, J = 5.5 Hz, 1H), |
| | yl)sulfonyl)azetidin-3- | | 7.54 (d, J = 10.7 Hz, 1H), 6.58 (s, 4H), 4.00 (dq, J = 13.0, |
| | yl)(methyl)amino)phenol | | 6.8 Hz, 3H), 3.70-3.59 (m, 2H), 3.17 (s, 1H), 2.46 (s, 3H) |
| 23 | 4-(methyl(1-((1- | m/z = | 1H NMR (499 MHz, DMSO) δ 8.85 (s, 1H), 7.92 (s, 1H), |
| | methyl-1H-indol-6- | 372[M + H]+ | 7.80 (d, J = 8.3 Hz, 1H), 7.66 (d, J = 3.0 Hz, 1H), 7.44 |
| | yl)sulfonyl)azetidin- | | (dd, J = 8.3, 1.6 Hz, 1H), 6.62 (dd, J = 3.0, 0.7 Hz, 1H), |
| | 3-yl)amino)phenol | | 6.57-6.52 (m, 2H), 6.52-6.48 (m, 2H), 3.91 (d, J = 2.1 |
| | | | Hz, 6H), 3.59-3.42 (m, 2H), 2.29 (s, 3H). |
| 24 | 4-((1-((1H-indol-4- | m/z = | 1H NMR (499 MHz, DMSO) δ 11.70 (s, 1H), 8.84 (s, |
| | yl)sulfonyl)azetidin-3- | 358[M + H]+ | 1H), 7.79 (d, J = 8.1 Hz, 1H), 7.59 (t, J = 2.7 Hz, 1H), |
| | yl)(methyl)amino)phenol | | 7.49 (d, J = 7.0 Hz, 1H), 7.32 (t, J = 7.8 Hz, 1H), 6.80 |
| | | | (s, 1H), 6.58-6.50 (m, 2H), 6.50-6.42 (m, 2H), 3.91 (d, |
| | | | J = 4.6 Hz, 3H), 3.53 (g, J = 4.0 Hz, 2H), 3.32 (s, 23H), |
| | | | 2.18 (s, 3H. |
| 26 | 4-((1R,5S)-3-((1H-indol- | m/z = 384 | — |
| | 6-yl)sulfonyl)-3,8- | [M + H]+ | |
| | diazabicyclo[3.2.1]octan- | | |
| | 8-yl)phenol | | |
| 27 | 4-((1-((1H-indol-5- | m/z = | 1H NMR (499 MHz, DMSO) δ 11.69 (s, 1H), 8.84 (s, |
| | yl)sulfonyl)azetidin-3- | 358[M + H]+ | 1H), 8.12-8.04 (m, 1H), 7.64 (d, J = 8.6 Hz, 1H), 7.59 |
| | yl)(methyl)amino)phenol | | (t, J = 2.7 Hz, 1H), 7.51 (dd, J = 8.6, 1.7 Hz, 1H), 6.69 |
| | | | (s, 1H), 6.62-6.41 (m, 4H), 3.88 (p, J = 4.6 Hz, 3H), |
| | | | 3.52-3.38 (m, 2H), 2.28 (s, 3H) |
| 28 | 4-((1R,5S)-3-((1H-indol- | m/z = | 1H NMR (499 MHz, DMSO) δ 11.62 (s, 1H), 8.59 (s, |
| | 6-yl)sulfonyl)-3,9- | 398[M + H]+ | 1H), 7.85-7.73 (m, 2H), 7.65 (t, J = 2.7 Hz, 1H), 7.28 |
| | diazabicyclo[3.3.1]nonan- | | (dd, J = 8.4, 1.6 Hz, 1H), 6.66-6.45 (m, 5H), 4.03 (d, J = |
| | 9-yl)phenol | | 7.1 Hz, 1H), 3.89 (s, 2H), 3.64 (d, J = 10.6 Hz, 2H), 2.56 |
| | | | (d, J = 8.3 Hz, 3H), 1.99 (s, 1H), 1.84 (dd, J = 12.6, 6.4 |
| | | | Hz, 2H), 1.63 (dd, J = 13.5, 5.4 Hz, 2H), 1.48 (s, 1H), |
| | | | 1.17 (t, J = 7.1 Hz, 1H) |
| 33 | 4-(4-((1H-indol-6- | m/z = 384 | 1H NMR (400 MHz, DMSO) δ 11.57 (s, 1H), 8.78 (s, |
| | yl)sulfonyl)-4,7- | [M + H]+ | 1H), 7.93 (s, 1H), 7.73 (d, J = 8.3 Hz, 1H), 7.63 (d, J = |
| | diazaspiro[2.5]octan- | | 3.1 Hz, 1H), 7.42 (dd, J = 8.5, 1.7 Hz, 1H), 6.61-6.51 |
| | 7-yl)phenol | | (m, 5H), 3.74 (t, J = 5.0 Hz, 2H), 2.88 (t, J = 5.1 Hz, 2H), |
| | | | 2.46 (s, 2H), 1.08 (s, 2H), 0.81-0.76 (m, 2H). |
| 34 | 4-((1R,5S)-9-((1H-indol- | m/z = 398.3 | 1H NMR (400 MHz, DMSO) δ 11.55 (s, 1H), 8.75 (s. |
| | 6-yl)sulfonyl)-3,9- | [M + H]+ | 1H), 7.93 (dt, J = 1.6, 0.7 Hz, 1H), 7.79-7.66 (m, 1H), |
| | diazabicyclo[3.3.1]nonan- | | 7.62 (dd, J = 3.0, 1.6 Hz, 1H), 7.45 (dd, J = 8.4, 1.7 Hz, |
| | 3-yl)phenol | | 1H), 6.75-6.58 (m, 4H), 6.56 (d, J = 3.1 Hz, 1H), 4.06 |
| | | | (dd, J = 11.0, 4.6 Hz, 2H), 3.42 (dd, J = 11.7, 1.7 Hz, |
| | | | 2H), 2.73 (dd, J = 11.5, 3.5 Hz, 2H), 2.47-2.32 (m, 1H), |
| | | | 1.79-1.66 (m, 4H), 1.47-1.33 (m. 1H) |
| 42 | (1R,5S)-3-((1H-indol- | m/z = | 1H NMR (499 MHz, DMSO) δ 11.66 (s, 1H), 8.79 (s, |
| | 6-yl)sulfonyl)-9- | 410[M + H]+ | 1H), 7.84-7.70 (m, 2H), 7.67 (t, J = 2.8 Hz, 1H), 7.28 |
| | (4-hydroxyphenyl)-3,9- | | (dd, J = 8.4, 1.7 Hz, 1H), 6.86-6.74 (m, 2H), 6.68-6.55 |
| | diazabicyclo[3.3.1]nonan- | | (m, 3H), 4.33 (d, J = 4.5 Hz, 2H), 3.57 (d, J = 10.7 Hz, |
| | 7-one | | 2H), 2.43 (d, J = 9.4 Hz, 2H), 2.25 (d, J = 15.8 Hz, 2H) |
| 46 | 4-(5-((1H-indol-6- | m/z = 398 | 1H NMR (400 MHz, DMSO) δ 11.57 (s, 1H), 8.85 (s, |
| | yl)sulfonyl)-5,8- | [M + H]+ | 1H), 7.94-7.89 (m, 1H), 7.76 (d, J = 8.4 Hz, 1H), 7.68-7.62 |
| | diazaspiro[3.5]nonan- | | (m, 1H), 7.42 (dd, J = 8.4, 1.7 Hz, 1H), 6.82-6.74 |
| | 8-yl)phenol | | (m, 2H), 6.68-6.62 (m, 2H), 6.59 (t, J = 2.8 Hz, 1H), |
| | | | 3.52-3.45 (m, 2H), 3.00-2.92 (m, 4H), 2.28 (q, J = |
| | | | 10.7 Hz, 2H), 1.93 (t, J = 10.0 Hz, 2H), 1.78-1.62 (m, |
| | | | 1H), 1.53 (d, J = 10.9 Hz, 1H) |
| 49 | 4-(1-((1H-indol-6- | m/z = | 1H NMR (499 MHz, DMSO) δ 11.58 (s, 1H), 8.76 (s, |
| | yl)sulfonyl)octahydro- | 398[M + H]+ | 1H), 7.91 (s, 1H), 7.78 (d, J = 8.4 Hz, 1H), 7.70-7.61 |
| | 4H-pyrrolo[3,2- | | (m, 1H), 7.46 (dd, J = 8.4, 1.6 Hz, 1H), 6.67-6.57 (m, |
| | b]pyridin-4-yl)phenol | | 1H), 6.53 (s, 4H), 3.66 (dt, J = 9.3, 5.8 Hz, 1H), 3.41 |
| | | | (dddd, J = 15.7, 12.2, 8.7, 4.7 Hz, 2H), 3.24-3.12 (m, |
| | | | 1H), 3.03-2.92 (m, 1H), 2.75 (t, J = 9.6 Hz, 1H), 1.86 |
| | | | (dd, J = 12.9, 4.5 Hz, 1H), 1.78-1.57 (m, 4H), 1.52-1.43 |
| | | | (m, 1H), 1.39 (dd, J = 11.0, 6.9 Hz, 1H) |

### Method D

### Synthesis of (S)-4-((1-((1H-indol-5-yl)sulfonyl)pyrrolidin-3-yl)(methyl)amino)phenol (Ex 30)

### Step 1: tert-butyl (S)-3-((4-(tert-butoxy)phenyl)amino)pyrrolidine-1-carboxylate

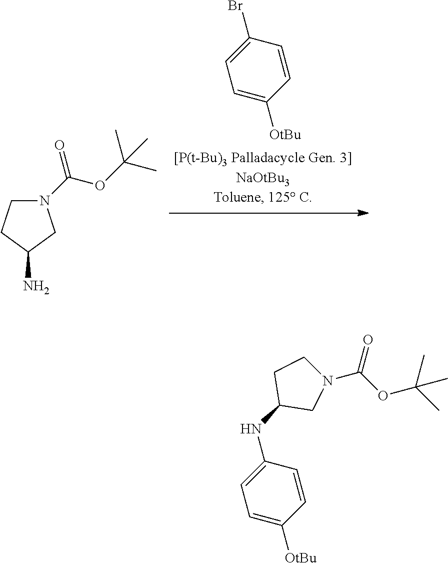 [SMILES: CC(C)(C)OC(=O)N1CC[C@H](N)C1 ; CC(C)(C)Oc1ccc(Br)cc1 ; CC(C)(C)OC(=O)N1CC[C@H](Nc2ccc(OC(C)(C)C)cc2)C1]

Added I-bromo-4-tert-butoxy-benzene (1.10 eq. 1038 mg, 4.53 mmol), sodium t-butanolate (2.00 eq. 795 mg, 8.27 mmol) RuPhos Pd G3 (0.0500 eq. 0.21 mL, 0.207 mmol) in seal tube and was degassed under argon three times. Toluene (8 mL) and (S)-3-Amino-1-N-Boc-pyrrolidine (1.00 eq, 0.72 mL, 4.13 mmol) were added and heated reaction at 125° C. overnight under argon balloon. The reaction mixture was filtered and rinsed with ethyl acetate. Then organic layer was evaporated, and product was purified by silica gel column with 0-100% ethyl acetate in hexane to yield the desired product (1265 mg, 91%). LC-MS: m/z=335 [M+H]⁺.

### Step 2: tert-butyl (S)-3-((4-(tert-butoxy)phenyl)(methyl)amino)pyrrolidine-1-carboxylate

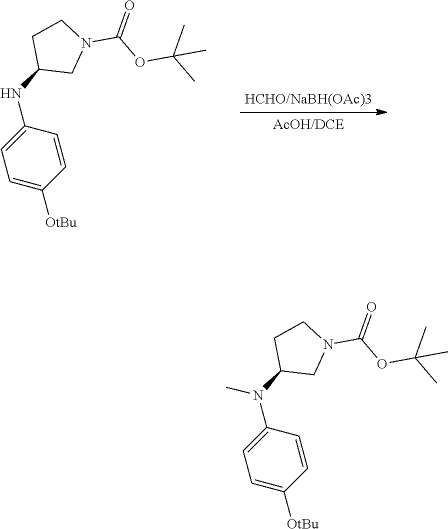 [SMILES: CC(C)(C)OC(=O)N1CC[C@H](Nc2ccc(OC(C)(C)C)cc2)C1 ; CN(c1ccc(OC(C)(C)C)cc1)[C@H]1CCN(C(=O)OC(C)(C)C)C1]

To a solution of tert-butyl rac-(3S)-3-(4-tert-butoxyanilino)pyrrolidine-1-carboxylate (1.00 eq, 780 mg, 2.33 mmol) in DCE (6 mL) was added formaldehyde (4.00 eq. 757 mg, 9.33 mmol), acetic acid (1.00 eq, 0.13 mL, 2.33 mmol) and then sodium tris(acetoxy)borohydride (4.00 eq, 1977 mg, 9.33 mmol). The reaction was stirred at 20° C. overnight. The reaction mixture was worked up with water and extracted with DCM. Then organic layer was evaporated, and product was purified by silica gel column with 0-100% ethyl acetate in hexane yielding the desired product (585 mg, 72%). LC-MS: m/z=349 [M+H]⁺.

### Step 3 & 4: (S)-4-methyl-(1-((1-triisopropylsilyl-1H-indol-5-yl)sulfonyl)pyrrolidin-3-yl)amino)phenol

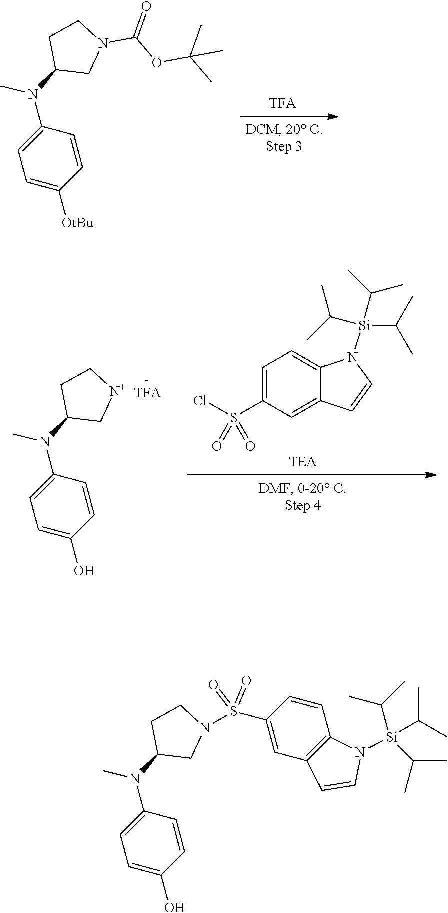 [SMILES: CN(c1ccc(OC(C)(C)C)cc1)[C@H]1CCN(C(=O)OC(C)(C)C)C1 ; CN(c1ccc(O)cc1)[C@H]1CC[NH2+]C1 ; CC(C)[Si](C(C)C)(C(C)C)n1ccc2cc(S(=O)(=O)Cl)ccc21 ; CC(C)[Si](C(C)C)(C(C)C)n1ccc2cc(S(=O)(=O)N3CC[C@H](N(C)c4ccc(O)cc4)C3)ccc21]

To a solution of tert-butyl rac-(3S)-3-(4-tert-butoxy-N-methyl-anilino)pyrrolidine-1-carboxylate (1.00 eq, 585 mg, 1.68 mmol) in DCM (4 mL) was added TFA (39.2 eq. 2.0 mL, 65.8 mmol) and the reaction mixture was stirred for 2 hours at 20° C. until completion. The reaction mixture was evaporated and dried under high vacuum overnight to isolate 885 mg product as an oil. Product was contained 42% TFA by weight and was used as crude in the next step. LC-MS: m/z=193 [M+H]⁺.

To a solution of 4-[methyl-[rac-(3S)-pyrrolidin-1-ium-3-yl]amino]phenol;2,2,2-trifluoroacetate (1.00 eq, 62 mg, 0.117 mmol) in DMF (2 mL) was added triethylamine (5.00 eq, 0.082 mL, 0.587 mmol) and 1-(tert-butyldimethylsilyl)-1H-indole-5-sulfonyl chloride (1.00 eq, 39 mg, 0.117 mmol) in portions at 0° C. The reaction mixture was stirred at 0° C. for 30 minutes to completion. Then reaction mixture was worked up with water and ethyl acetate. The organic layer was washed twice with water (2×4 ml). The residue was subjected to column chromatography on silica gel, eluting with ethyl acetate-hexane to provide the desired product (35 mg, 61%). LC-MS: m/z=486 [M+H]⁺.

### Step 5: (S)-4-((1-((1H-indol-5-yl)sulfonyl)pyrrolidin-3-yl)(methyl)amino)phenol

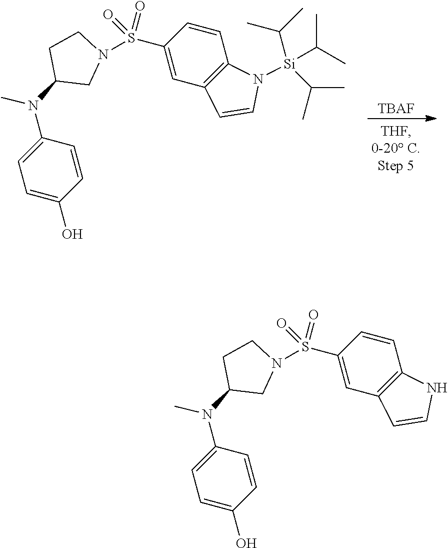

To a solution of 4-[methyl-[rac-(3S)-1-[1-[tert-butyl(dimethyl)silyl]indol-5-yl]sulfonylpyrrolidin-3-yl]amino]phenol (1.00 eq. 35 mg, 0.0721 mmol) in THF (3 mL) was added 0.072 ml of 2N of TBAF (2.00 eq, 5.3 mg, 0.144 mmol) at 0° C. The reaction mixture was stirred to 20° C. for 2 hours until completion. Water (4 ml) and ethyl acetate (4 ml) was added, and mixture was extracted with ethyl acetate. The organic layer was dried and concentrated. The residue was subjected to column chromatography on silica gel, eluting with ethyl acetate and hexane to isolate product (24 mg, 90%). LC-MS: m/z=372 [M+H]⁺. 1H NMR (499 MHz, DMSO) δ 11.64 (s, 1H), 8.84 (s, 1H), 8.05 (d, J=1.6 Hz, 1H), 7.61 (d, J=8.5 Hz, 1H), 7.59-7.54 (m, 1H), 7.50 (dd, J=8.6, 1.8 Hz, 1H), 6.72-6.65 (m, 1H), 6.66-6.60 (m, 2H), 6.61-6.52 (m, 2H), 3.77 (p, J=7.2 Hz, 1H), 3.33-3.26 (m, 33H), 3.23 (dd, J=10.2, 7.4 Hz, 1H), 3.06 (dt, J=9.8, 7.8 Hz, 1H), 2.96 (dd, J=10.2, 6.5 Hz, 1H), 2.44 (s, 3H), 1.84 (dtd, J=11.7, 7.3, 4.3 Hz, 1H), 1.65 (dq, J=12.6, 8.1 Hz, 1H). The following examples were prepared using the same procedure:

| | | LC/MS | |
|---|---|---|---|
| Ex. | Name | (ES, m/z) | ¹H-NMR |
| 15 | 4-((1-((1H-indol-6- | m/z = 386.4 | 1H NMR (400 MHz, DMSO) δ 11.61 (s, 1H), 8.61 (s, |
| | yl)sulfonyl)piperidin-4- | [M + H]+ | 1H), 7.98-7.73 (m, 2H), 7.67 (t, J = 2.7 Hz, 1H), 7.34 |
| | yl)(methyl)amino)phenol | | (dd, J = 8.3, 1.6 Hz, 1H), 6.85-6.24 (m, 5H), 3.71 (d, |
| | | | J = 11.5 Hz, 2H), 2.40-2.21 (m, 2H), 1.62 (d, J = 3.5 |
| | | | Hz, 4H) |
| 18 | 4-((1-((1H-indazol-6- | m/z = 358.8 | 1H NMR (400 MHz, DMSO) δ 13.59 (s, 1H), 8.85 (s, |
| | yl)sulfonyl)azetidin-3- | [M + H]+ | 1H), 8.32 (t, J = 1.3 Hz, 1H), 8.13-7.95 (m, 2H), 7.51 |
| | yl)(methyl)amino)phenol | | (dd, J = 8.5, 1.5 Hz, 1H), 6.64-6.44 (m, 4H), 4.03-3.88 |
| | | | (m, 3H), 3.60-3.51 (m, 2H), 2.32 (s, 3H) |
| 20 | rac-(R)-4-((1-((1H-indol- | m/z = 372 | 1H NMR (499 MHz, DMSO) δ 11.61 (s, 1H), 8.84 (s, |
| | 6-yl)sulfonyl)pyrrolidin- | [M + H]+ | 1H), 7.86 (s, 1H), 7.77 (d, J = 8.3 Hz, 1H), 7.73-7.63 |
| | 3-yl)(methyl)amino)phenol | | (m, 1H), 7.39 (dd, J = 8.3, 1.6 Hz, 1H), 6.70-6.46 (m, |
| | | | 5H), 3.78 (p, J = 7.1 Hz, 1H), 3.38(m, 1H), 3.29-3.16 |
| | | | (m, 1H), 3.07 (dt, J = 9.8, 7.8 Hz, 1H), 2.97 (dd, J = |
| | | | 10.2, 6.5 Hz, 1H), 2.44 (s, 3H), 1.85 (dtd, J = 11.7, |
| | | | 7.3, 4.3 Hz, 1H), 1.66 (dq, J = 12.7, 8.2 Hz, 1H). |
| 30 | rac-(R)-4-((1-((1H-indol- | m/z = 372 | 1H NMR (499 MHz, DMSO) δ 11.64 (s, 1H), 8.84 (s, |
| | 5-yl)sulfonyl)pyrrolidin- | [M + H]+ | 1H), 8.05 (d, J = 1,6 Hz, 1H), 7.61 (d, J = 8.5 Hz, 1H), |
| | 3-yl)(methyl)amino)phenol | | 7.59-7.54 (m, 1H), 7.50 (dd, J = 8.6, 1.8 Hz, 1H), |
| | | | 6.72-6.65 (m, 1H), 6.66-6.60 (m, 2H), 6.61-6.52 (m, |
| | | | 2H), 3.77 (p, J = 7.2 Hz, 1H), 3.33-3.26 (m, 33H), |
| | | | 3.23 (dd, J = 10.2, 7.4 Hz, 1H), 3.06 (dt, J = 9.8, 7.8 Hz, |
| | | | 1H), 2.96 (dd, J = 10.2, 6.5 Hz, 1H), 2.44 (s, 3H), 1.84 |
| | | | (dtd, J = 11.7, 7.3, 4.3 Hz, 1H), 1.65 (dq, J = 12.6, 8.1 |
| | | | Hz, 1H) |
| 31 | rac-(R)-4-((1-((1H-indol- | m/z = 372 | 1H NMR (499 MHz, DMSO) δ 11.66 (s, 1H), 8.83 (s, |
| | 4-yl)sulfonyl)pyrrolidin- | [M + H]+ | 1H), 7.75 (d, J = 8.1 Hz, 1H), 7.58 (t, J = 2.8 Hz, 1H), |
| | 3-yl)(methyl)amino)phenol | | 7.47 (d, J = 7.2 Hz, 1H), 7.28 (t, J = 7.8 Hz, 1H), 6.83 |
| | | | (s, 1H), 6.57 (s, 4H), 3.80-3.64 (m, 1H), 3.32-3.24 |
| | | | (m, 2H), 3.17-3.08 (m, 1H), 3.01 (dd, J = 10.3, 6.5 Hz, |
| | | | 1H), 2.38 (s, 3H), 1.85-1.75 (m, 1H), 1.69-1.55 (m, 1H). |
| 32 | rac-(R)-4-((1-((2-chloro- | m/z = 424 | 1H NMR (499 MHz, MeOD) δ 7.92 (d, J = 5.5 Hz, 1H), |
| | 5-fluoro-1H-indol-6- | [M + H]+ | 7.61 (s, 1H), 7.41 (d, J = 10.8 Hz, 1H), 6.83-6.74 (m, |
| | yl)sulfonyl)pyrrolidin- | | 2H), 6.69-6.59 (m, 2H), 3.75 (p, J = 6.8 Hz, 1H), 3.53 |
| | 3-yl)(methyl)amino)phenol | | (td, J = 9.0, 4.4 Hz, 1H), 3.44 (dd, J = 10.2, 6.9 Hz, 1H), |
| | | | 3.15 (dd, J = 10.2, 6.6 Hz, 1H), 2.58 (s, 3H), 1.93-1.79 |
| | | | (m, 1H) |
| 37 | rac-4-(((3R,5S)-1-((1H- | LC-MS: m/z = | H NMR (499 MHz, DMSO) δ 11.63 (s, 1H), 8.82 (s, |
| | indol-6-yl)sulfonyl)- | 386 [M + H]+ | 1H), 7.89 (s, 1H), 7.80 (d, J = 8.4 Hz, 1H), 7.69 (t, J = |
| | 5-methylpyrrolidin-3- | | 2.8 Hz, 1H), 7.42 (dd, J = 8.3, 1.6 Hz, 1H), 6.63 (t, J = |
| | yl)(methyl)amino)phenol | | 2.0 Hz, 1H), 6.59-6.44 (m, 2H), 6.44-6.33 (m, 2H), |
| | | | 3.55 (m, 4H), 3.08 (s, 1H), 2.99-2.80 (m, 1H), 1.98 (s, |
| | | | 1H), 1.47 (dt, J = 12.1, 9.9 Hz, 1H), 1.35 (d, J = 6.1 Hz, |
| | | | 3H), 1.17 (s, 1H). |
| 38 | rac-(R)-4-((4-((1H- | LC-MS: m/z = | 1H NMR (499 MHz, DMSO) δ 11.60 (s, 1H), 8.72 (s, |
| | indol-6-yl)sulfonyl)-4- | 398 [M + H]+ | 1H), 7.84 (s, 1H), 7.77 (d, J = 8.3 Hz, 1H), 7.66 (t, J = |
| | azaspiro[2.4]heptan-6- | | 2.8 Hz, 1H), 7.37 (dd, J = 8.3, 1.6 Hz, 1H), 6.61 (s, 1H), |
| | yl)(methyl)amino)phenol | | 6.58-6.48 (m, 4H), 3.83 (dd, J = 6.9, 2.9 Hz, 1H), 3.18 |
| | | | (dd, J = 10.5, 3.0 Hz, 1H), 2.96 (d, J = 9.7 Hz, 1H), 2.46 |
| | | | (s, 3H), 0.69 (dt, J = 10.5, 5.3 Hz, 1H), 0.62-0.54 (m, |
| | | | 1H), 0.54-0.41 (m, 2H) |
| 39 | rac-4-(((3R,4S)-1-((1H- | m/z = 386 | 1H NMR (499 MHz, DMSO) δ 11.60 (s, 1H), 8.70 (s, |
| | indol-6-yl)sulfonyl)-4- | [M + H]+ | 1H), 7.87 (s, 1H), 7.78 (d, J = 8.3 Hz, 1H), 7.67 (t, J = |
| | methylpyrrolidin-3- | | 2.8 Hz, 1H), 7.41 (dd, J = 8.3, 1.6 Hz, 1H), 6.64-6.59 |
| | yl)(methyl)amino)phenol | | (m, 1H), 6.54 (s, 4H), 3.54-3.39 (m, 2H), 3.24 (d, J = |
| | | | 2.4 Hz, 1H), 3.13-2.99 (m, 1H), 2.68 (t, J = 9.6 Hz, |
| | | | 1H), 2.47 (s, 3H), 2.09 (dt, J = 16.0, 8.9 Hz, 1H), 0.76 |
| | | | (d, J = 6.6 Hz, 3H) |
| 48 | rac-4-(((3R,5R)-1-((1H- | m/z = | 1H NMR (499 MHz, DMSO) δ 11.56 (s, 1H), 8.91 (s, |
| | indol-6-yl)sulfonyl)-5- | 386[M + H]+ | 1H), 7.86 (s, 1H), 7.75 (d, J = 8.3 Hz, 1H), 7.69-7.59 |
| | methylpyrrolidin-3- | | (m, 1H), 7.40 (dd, J = 8.3, 1.6 Hz, 1H), 6.73-6.65 (m, |
| | yl)(methyl)amino)phenol | | 2H), 6.62-6.52 (m, 3H), 3.94 (d, J = 8.1 Hz, 1H), 3.71 |
| | | | (d, J = 6.4 Hz, 1H), 3.46 (dd, J = 9.6, 6.6 Hz, 1H), 2.85 |
| | | | (dd, J = 9.5, 8.1 Hz, 1H), 2.28 (s, 3H), 1.62-1.42 (m, |
| | | | 2H), 1.25 (d, J = 6.4 Hz, 3H). |

### Example 10: N-[1-(4-hydroxyphenyl)azetidin-3-yl]-1H-indole-6-sulfonamide

### Step 1: 1-benzyloxy-4-bromo-benzene as a white powder

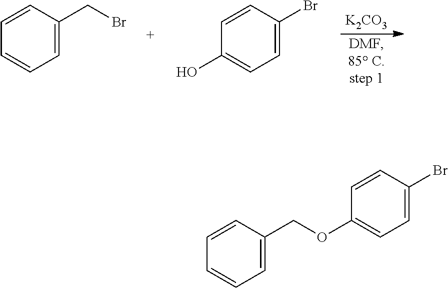

A mixture of 4-bromophenol (98%, 5.10 g, 28.9 mmol), K₂CO₃ (7.99 g, 57.8 mmol), and benzyl bromide (3.8 mL, 31.8 mmol) in acetone (41 mL) was heated-up to reflux and stirred for 5 h. The reaction mixture was cooled down to RT and filtered. The filtrate was concentrated under reduced pressure to afford 1-benzyloxy-4-bromo-benzene as a white powder (7.58 g, 100%). ¹H NMR (400 MHz, DMSO) δ 7.48-7.30 (m, 7H), 7.03-6.94 (m, 2H), 5.10 (s, 2H).

### Step 2: tert-butyl N-[1-(4-benzyloxyphenyl)azetidin-3-yl]carbamate

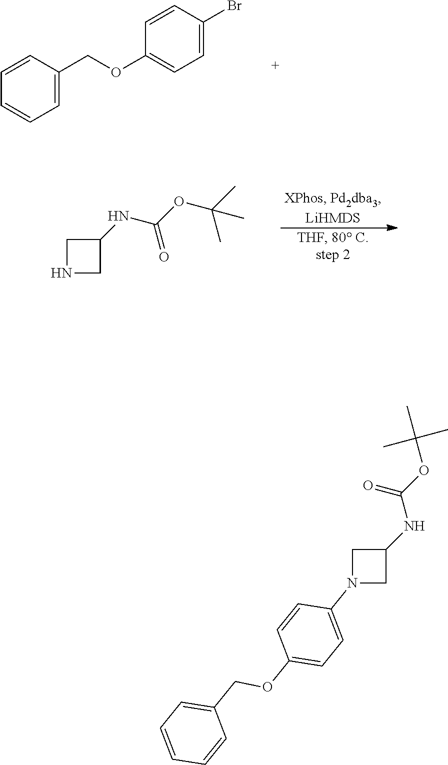 [SMILES: Brc1ccc(OCc2ccccc2)cc1 ; CC(C)(C)OC(=O)NC1CNC1 ; "THF, 80° C.step 2" ; "XPhos, Pd2dba3,LiHMDS" ; CC(C)(C)OC(=O)NC1CN(c2ccc(OCc3ccccc3)cc2)C1]

The title compound was synthesized in a similar manner to 1^{st} step of Example 17 (Buchwald reaction), using 1-benzyloxy-4-bromo-benzene and tert-butyl azetidin-3-ylcarbamate, and was isolated as a white fluffy solid (312 mg, 31%) after purification by flash chromatography (50 g silica gel, 0-60% EtOAc in heptane) then trituration in pentane. LC-MS: m/z=355 [M+H]⁺.

### Step 3: tert-butyl N-[1-(4-hydroxyphenyl)azetidin-3-yl]carbamate

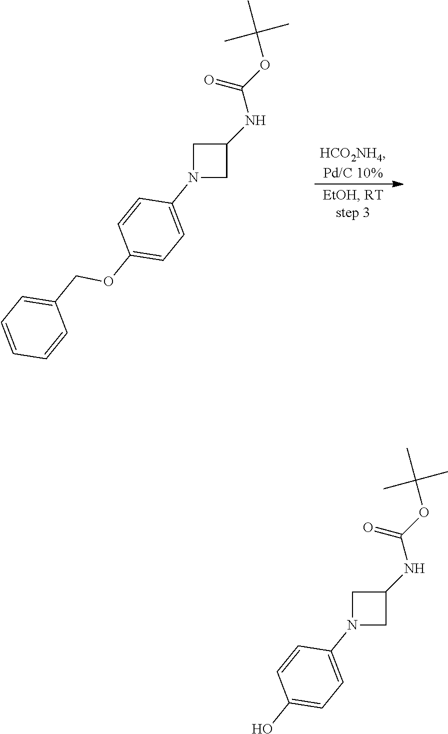 [SMILES: CC(C)(C)OC(=O)NC1CN(c2ccc(OCc3ccccc3)cc2)C1 ; "EtOH, RTstep 3" ; "HCO2NH4,Pd/C 10%" ; CC(C)(C)OC(=O)NC1CN(c2ccc(O)cc2)C1]

To a solution of tert-butyl N-[1-(4-benzyloxyphenyl)azetidin-3-yl]carbamate (447 mg, 1.26 mmol) in Ethanol (28 mL), ammonium formate (0.75 mL, 15.2 mmol) and palladium on carbon (10 wt. % loading, 207 mg, 0.195 mmol) were added under nitrogen. The reaction mixture was flushed for 15 min then heated-up to 80° C. and stirred for 3.5 h. The reaction was cooled down to RT and filtered through a celite pad and the filtrate was concentrated under reduced pressure to afford tert-butyl N-[1-(4-hydroxyphenyl)azetidin-3-yl]carbamate (356 mg, 93%) as a pale brown gum. LC-MS: m/z=265 [M+H]⁺.

### Step 4: 4-(3-aminoazetidin-1-yl)phenol (TFA salt)

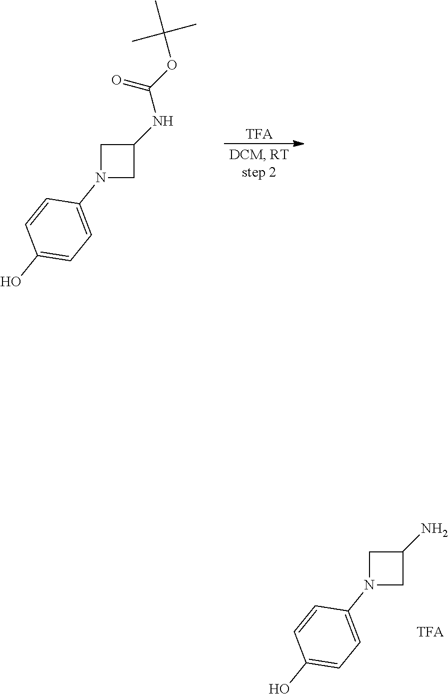 [SMILES: CC(C)(C)OC(=O)NC1CN(c2ccc(O)cc2)C1 ; "DCM, RTstep 2" ; "TFA" ; NC1CN(c2ccc(O)cc2)C1]

Tert-butyl N-[1-(4-hydroxyphenyl)azetidin-3-yl]carbamate (356 mg) in 2 ml DCM was treated with 1 ml TFA. After 2 hrs at RT the solution was concentrated under vacuum yielding 335 mg of brown powder (73%). LC-MS: m/z=165 [M+H]⁺.

### Step 5: N-[1-(4-hydroxyphenyl)azetidin-3-yl]-1H-indole-6-sulfonamide

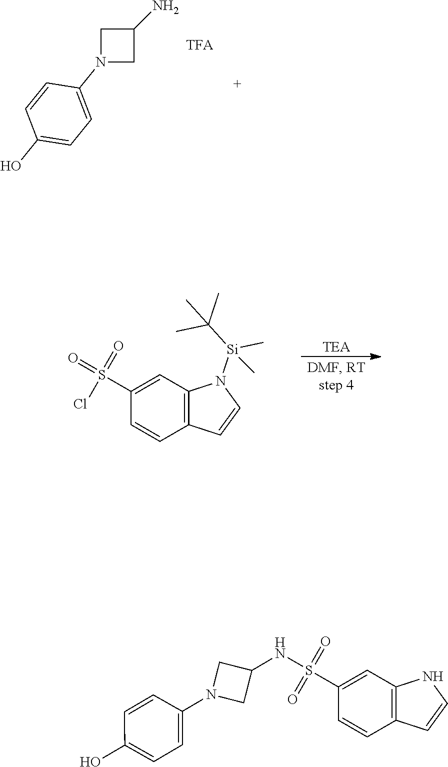 [SMILES: NC1CN(c2ccc(O)cc2)C1 ; CC(C)(C)[Si](C)(C)n1ccc2ccc(S(=O)(=O)Cl)cc21 ; "DMF, RTstep 4" ; "TEA" ; O=S(=O)(NC1CN(c2ccc(O)cc2)C1)c1ccc2cc[nH]c2c1]

The title compounds were synthesized in a similar manner to general Method A (sulfonylation), using 4-(3-aminoazetidin-1-yl)phenol (TFA salt) and 1-(tert-butyldimethylsilyl)-1H-indole-6-sulfonyl chloride, and was isolated as a white powder (89 mg, 35%) after purification by flash chromatography (Silica 12 g, 10-40% EtOAc in heptane then 0-4% MeOH in DCM). LC-MS: m/z=343 [M+H]⁺. ¹H NMR (400 MHz, DMSO) δ 11.60 (s, 1H), 8.58 (s, 1H), 8.11 (d, J=8.2 Hz, 1H), 7.92-7.87 (m, 1H), 7.74 (d, J=8.3 Hz, 1H), 7.65 (t, J=2.7 Hz, 1H), 7.42 (dd, J=8.4, 1.7 Hz, 1H), 6.62-6.50 (m, 4H), 6.20-6.11 (m, 2H), 4.08 (d, J=6.9 Hz, 1H), 3.74 (t, J=7.2 Hz, 2H), 3.22 (dd, J=7.4, 6.2 Hz, 2H).

### Example 11: 4-[[1-(1H-indol-6-ylsulfonyl)azetidin-3-yl]amino]phenol

### Step 1: N-(4-(tert-butoxy)phenyl)azetidin-3-amine

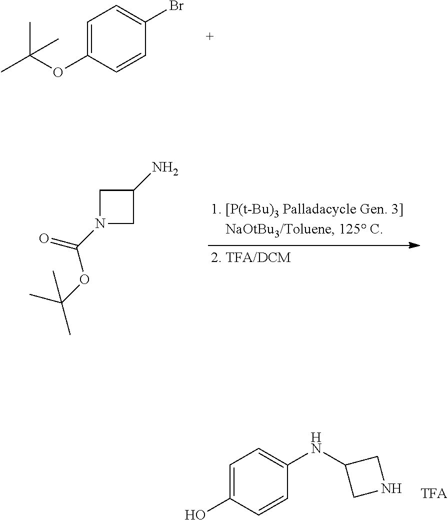

1-bromo-4-tert-butoxy-benzene (1.10 eq, 1038 mg, 4.53 mmol), sodium t-butanolate (2.00 eq, 795 mg, 8.27 mmol) RuPhos Pd G3 (0.05 eq, 0.21 mL, 0.207 mmol) were loaded in a seal tube and was degassed under argon three times. Toluene (8 mL) and (S)-3-Amino-1-N-Boc-pyrrolidine (1.00 eq. 0.72 mL, 4.13 mmol) were added and heated reaction at 125° C. overnight under argon balloon. The reaction mixture was filtered and rinsed with ethyl acetate. Then organic layer was evaporated, and product was purified by silica gel column with 0-100% ethyl acetate in hexane to yield the desired product (1265 mg, 91%). LC-MS: m/z=321 [M+H]⁺.

The crude was dissolved in 10 ml of DCM and was treated with 5 ml of TFA. After 2 hrs at RT the solvent was concentrated under vacuum yielding 1300 mg of desired intermediate N-(4-(tert-butoxy)phenyl)azetidin-3-amine as TFA salt.

### Step 2: 4-[[1-[1-[tert-butyl(dimethyl)silyl]indol-6-yl]sulfonylazetidine-3-yl]amino]phenol

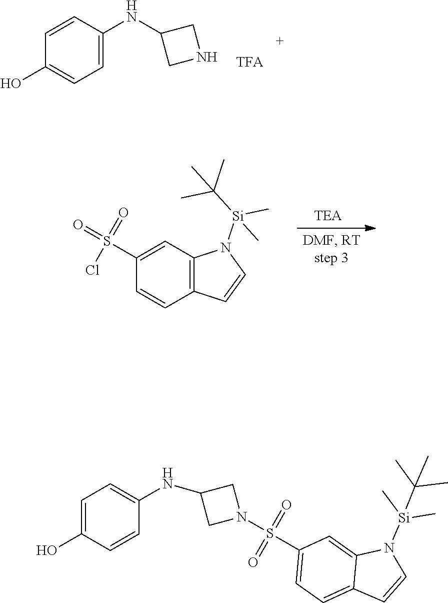 [SMILES: Oc1ccc(NC2CNC2)cc1 ; CC(C)(C)[Si](C)(C)n1ccc2ccc(S(=O)(=O)Cl)cc21 ; CC(C)(C)[Si](C)(C)n1ccc2ccc(S(=O)(=O)N3CC(Nc4ccc(O)cc4)C3)cc21]

To a solution of 4-(azetidin-3-ylamino) (100 mg, 0.359 mmol) in DMF (1.8 mL) and triethylamine (0.13 mL, 0.899 mmol) was added 1-(tert-butyldimethylsilyl)-1H-indole-6-sulfonyl chloride (95%, 100 mg, 0.288 mmol). The mixture was stirred at RT for 2 h. The mixture was diluted with EtOAc and washed with sat. NaHCO₃ solution. The organic layer was dried over sodium sulfate and concentrated in vacuo to afford the desired product which was used without further purification (164 mg, 37%). LC-MS: m/z=458.0 [M+H]⁺.

### Step 3: 4-[[1-(1H-indol-6-ylsulfonyl)azetidin-3-yl]amino]phenol

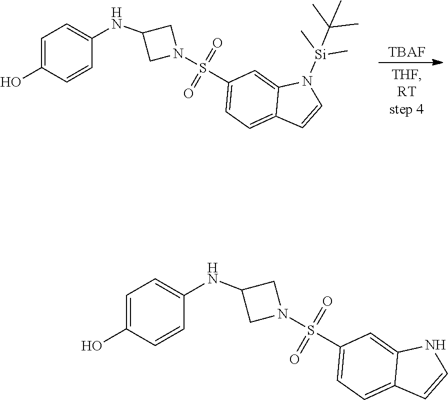

To a solution of 4-[[l-[l-[tert-butyl(dimethyl)silyl]indol-6-yl]sulfonylazetidine-3-yl]amino]phenol (155 mg, 0.338 mmol) in THF (1.6 mL), 1M TBAF (0.41 mL, 0.406 mmol) was added and the mixture was stirred at RT for 30 min. A precipitate was formed and filtered. The crude material was purified through a silica gel column with 0-10% methanol in DCM, and lyophilized to afford 4-[[1-(1H-indol-6-ylsulfonyl)azetidin-3-yl]amino]phenol (25.5 mg, 21%). LC-MS: m/z=343.9 [M+H]⁺. ¹H NMR (400 MHz, DMSO) δ 11.65 (s, 1H), 8.45 (s, 1H), 7.88 (dt, J=1.6, 0.7 Hz, 1H), 7.81 (d, J=8.3 Hz, 1H), 7.69 (t, J=2.6 Hz, 1H), 7.40 (dd, J=8.4, 1.6 Hz, 1H), 6.69-6.55 (m, 1H), 6.55-6.38 (m, 2H), 6.29-6.15 (m, 2H), 5.42 (d, J=6.5 Hz, 1H), 3.99 (dd, J=7.8, 6.7 Hz, 2H), 3.87 (h, J=6.3 Hz, 1H), 3.37 (dd, J=7.7, 6.0 Hz, 2H).

### Example 12: N-[1-(4-hydroxyphenyl)-4-piperidyl]-N-methyl-1H-indole-6-sulfonamide

### Step 1: tert-butyl N-[1-(4-benzyloxyphenyl)-4-piperidyl]-N-methyl-carbamate

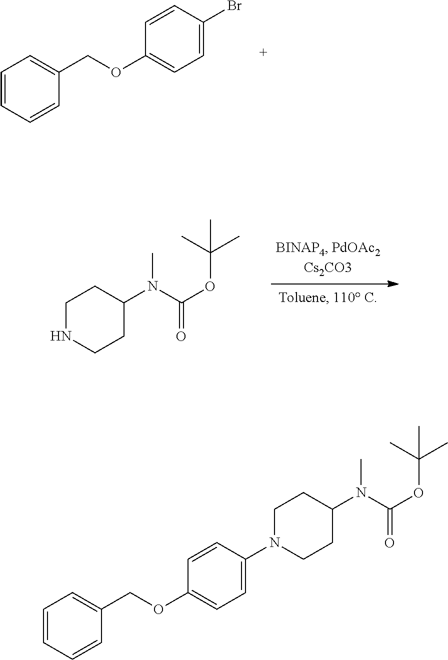 [SMILES: Brc1ccc(OCc2ccccc2)cc1 ; CN(C(=O)OC(C)(C)C)C1CCNCC1 ; CN(C(=O)OC(C)(C)C)C1CCN(c2ccc(OCc3ccccc3)cc2)CC1]

A sealed vial was successively charged with tert-butyl N-methyl-N-(4-piperidyl)carbamate (556 mg, 2.60 mmol), I-bromo-4-tert-butoxybenzene (615 mg, 2.60 mmol), cesium carbonate (2121 mg, 6.51 mmol) and BINAP (97%, 167 mg, 0.260 mmol) in anhydrous toluene (15 mL). The mixture was purged with argon for 5 min and Pd(OAc)₂ (60 mg, 0.260 mmol) was added. The reaction mixture was stirred at 90° C. for 18 h then diluted with water. The aqueous layer was extracted twice with EtOAc. The combined organic layers were washed with brine then dried over a phase separator and concentrated under reduced pressure. The crude material was purified by flash chromatography (25 g silica gel column, 0-50% EtOAc in heptane eluent)/the appropriate fractions were gathered and concentrated under reduced pressure to tert-butyl N-[1-(4-benzyloxyphenyl)-4-piperidyl]-N-methyl-carbamate (440 mg, 34%) as an off-white solid. LC-MS: m/z=297 [M+H-^{t}Bu]⁺.

### Step 2:tert-butyl N-[1-(4-hydroxyphenyl)-4-piperidyl]-N-methyl-carbamate

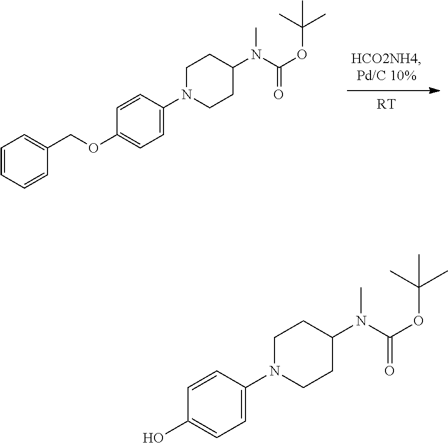 [SMILES: CN(C(=O)OC(C)(C)C)C1CCN(c2ccc(OCc3ccccc3)cc2)CC1 ; "RT" ; CN(C(=O)OC(C)(C)C)C1CCN(c2ccc(O)cc2)CC1]

To a solution of tert-butyl N-[1-(4-benzyloxyphenyl)-4-piperidyl]-N-methyl-carbamate (409 mg, 1.03 mmol) in ethanol (10 mL), palladium (10%, 220 mg, 0.206 mmol) and ammonium formate (0.51 mL, 10.3 mmol). The reaction mixture was heated up to 75° C. and stirred for 1 h. The reaction mixture was cooled down and filtered through celite pad. The precipitate was triturated and washed with ethanol and the filtrate was concentrated under reduced pressure to dryness to afford the title compound as a yellow powder (360 mg, 100%). LC-MS: m/z=307.2 [M+H]⁺.

### Step 3: 4-[4-(methylamino)-1-piperidyl]phenyl hydrochloride

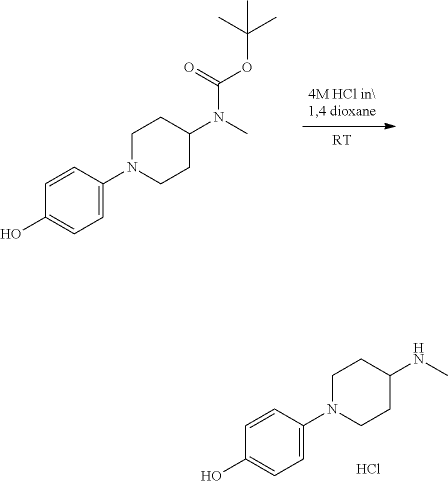 [SMILES: CN(C(=O)OC(C)(C)C)C1CCN(c2ccc(O)cc2)CC1 ; "RT" ; CNC1CCN(c2ccc(O)cc2)CC1 ; Cl]

To a solution of tert-butyl N-[l-(4-hydroxyphenyl)-4-piperidyl]-N-methyl-carbamate (320 mg, 1.04 mmol) in anhydrous 1,4-dioxane (7 mL) was added a solution of hydrogen chloride (4.0M in 1,4-dioxane, 2.6 mL, 10.4 mmol). The reaction mixture was stirred at RT for 18 h. The reaction mixture was filtered, and the residue was rinsed with 1,4-dioxane and n-pentane, and dried under reduced pressure to afford 4-[4-(methylamino)-1-piperidyl]phenol hydrochloride as an off-white powder (249 mg, 93%). LC-MS: m/z=207.4 [M+H]⁺.

### Step 4: 1-[tert-butyl(dimethyl)silyl]-N-[1-(4-hydroxyphenyl)-4-piperidyl]-N-methyl-indole-6-sulfonamide

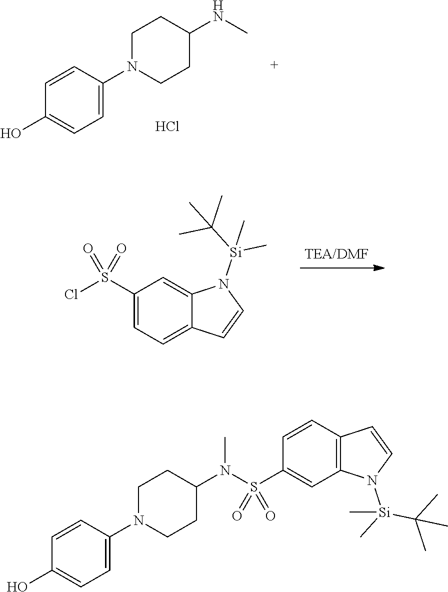 [SMILES: CNC1CCN(c2ccc(O)cc2)CC1 ; Cl ; CC(C)(C)[Si](C)(C)n1ccc2ccc(S(=O)(=O)Cl)cc21 ; CN(C1CCN(c2ccc(O)cc2)CC1)S(=O)(=O)c1ccc2ccn([Si](C)(C)C(C)(C)C)c2c1]

To a solution of 4-[4-(methylamino)-1-piperidyl]phenyl hydrochloride (34 mg, 0.14 mmol) in anhydrous DMF (1 mL), were added triethylamine (0.038 mL, 0.28 mmol) then 1-(tert-butyldimethylsilyl)-1H-indole-6-sulfonyl chloride (95%, 46 mg, 0.14 mmol). The reaction was stirred at RT for 30 min then diluter with EtOAc and sat. NaHCO₃. The layers were separated, the aqueous layer was extracted with EtOAc and the combined organic layers were dried over sodium sulfate, filtered, and concentrated under reduced pressure. The crude material was purified by flash chromatography (5 g silica gel column, 0-80% EtOAc in heptane eluent). The appropriate fractions were combined, concentrated under reduced pressure. The resulting gum was lyophilized overnight to afford 1-[tert-butyl(dimethyl)silyl]-N-[1-(4-hydroxyphenyl)-4-piperidyl]-N-methyl-indole-6-sulfonamide (48 mg, 55%) as a pale brown oil. LCMS: m/z=500 [M+H]⁺.

### Step 5: N-[1-(4-hydroxyphenyl)-4-piperidyl]-N-methyl-1H-indole-6-sulfonamide

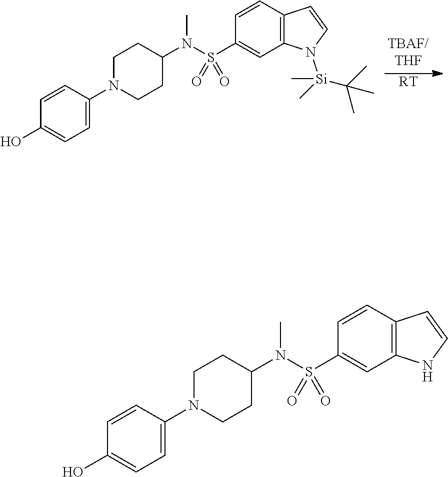

To a solution of 1-[tert-butyl(dimethyl)silyl]-N-[1-(4-hydroxyphenyl)-4-piperidyl]-N-methyl-indole-6-sulfonamide (48 mg, 0.096 mmol) in anhydrous THF (0.5 mL), was added a solution of TBAF (1.0M in THF, 0.11 mL, 0.11 mmol). The reaction was stirred for 2 h at RT then diluted with EtOAc and sat. NaHCO₃. Aqueous layer was washed three times with ethyl acetate and the combined organic layers were washed with brine, dried using a phase separator and concentrated under reduced pressure. The crude material was purified through a silica gel column with 0-20% methanol in dichloromethane and lyophilized to afford N-[1-(4-hydroxyphenyl)-4-piperidyl]-N-methyl-1H-indole-6-sulfonamide (22 mg, 58%). LC-MS: m/z=386.5 [M+H]⁺. ¹H NMR (400 MHz, DMSO) δ 11.56 (s, 1H), 8.79 (s, 1H), 7.88 (dt, J=1.7, 0.8 Hz, 1H), 7.75 (d, J=8.1 Hz, 1H), 7.67-7.58 (m, 1H), 7.41 (dd, J=8.4, 1.7 Hz, 1H), 6.81-6.67 (m, 2H), 6.66-6.53 (m, 3H), 3.79 (tt, J=12.1, 3.9 Hz, 1H), 3.37 (d, J=11.7 Hz, 2H), 2.71 (s, 3H), 2.55 (d, J=2.3 Hz, 2H), 1.68 (qd, J=12.2, 4.0 Hz, 2H), 1.30 (d, J=12.1 Hz, 2H).

### Example 13: 4-[1[-(1H-indol-6-ylsulfonyl)azetidin-3-yl]-methyl-amino]phenol

The title compound was synthesized as described in Example 11, starting with tert-butyl 3-(methylamino)azetidine-1-carboxylate (35 mg, 0.19 mmol) and was isolated as white solid (43 mg, 63%) after purification by flash chromatography (silica gel column with 0-10% methanol in dichloromethane) and lyophilization. LC-MS: m/z=358.4 [M+H]⁺. ¹H NMR (400 MHz, DMSO) δ 11.63 (s, 1H), 8.83 (s, 1H), 7.88 (dt, J=1.6, 0.7 Hz, 1H), 7.70 (d, J=3.1 Hz, 1H), 7.41 (dd, J=8.3, 1.7 Hz, 1H), 6.64 (d, J=3.0 Hz, 1H), 6.59-6.31 (m, 4H), 5.76 (s, OH), 4.05-3.80 (m, 3H), 3.49 (q, J=2.5 Hz, 2H), 2.30 (s, 3H).

### Example 16: N-[1-(4-hydroxyphenyl)azetidin-3-yl]-N-methyl-1H-indole-6-sulfonamide

### Step 1: tert-butyl (1-(4-(tert-butoxy)phenyl)azetidin-3-yl)(methyl)carbamate

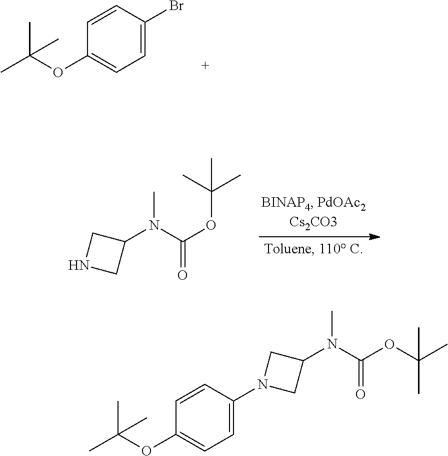 [SMILES: CC(C)(C)Oc1ccc(Br)cc1 ; CN(C(=O)OC(C)(C)C)C1CNC1 ; CN(C(=O)OC(C)(C)C)C1CN(c2ccc(OC(C)(C)C)cc2)C1]

A sealed vial was successively charged with tert-butyl N-methyl-N-(azetidine)carbamate (556 mg, 2.60 mmol), 1-bromo-4-tert-butoxybenzene (615 mg, 2.60 mmol), cesium carbonate (2121 mg, 6.51 mmol) and BINAP (97%, 167 mg, 0.260 mmol) in anhydrous toluene (15 mL). The mixture was purged with argon for 5 min and Pd(OAc)₂ (98%, 60 mg, 0.260 mmol) was added. The reaction mixture was stirred at 90° C. for 18 h then diluted with water. The aqueous layer was extracted twice with EtOAc. The combined organic layers were washed with brine then dried over a phase separator and concentrated under reduced pressure. The crude material was purified by flash chromatography (25 g silica gel column, 0-50% EtOAc in heptane eluent)/the appropriate fractions were gathered and concentrated under vacuum yielding tert-butyl (1-(4-(tert-butoxy)phenyl)azetidin-3-yl)(methyl)carbamate used as is for the next step. LC-MS: m/z (NH)=335 [M+H]⁺.

### Step 2: 4-(3-(methylamino)azetidin-1-yl)phenol hydrochloride

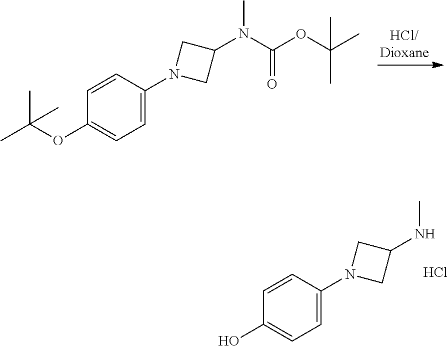 [SMILES: CN(C(=O)OC(C)(C)C)C1CN(c2ccc(OC(C)(C)C)cc2)C1 ; CNC1CN(c2ccc(O)cc2)C1 ; Cl]

A solution of HCl (4.0M in dioxane, 11 mL, 45.1 mmol) was added at RT to neat tert-butyl (1-(4-(tert-butoxy)phenyl)azetidin-3-yl)(methyl)carbamate (95%, 680 mg, 2.22 mmol). The reaction mixture was stirred for 18 h then filtered. The solid was washed with Et₂O then dried under reduced pressure to afford 4-(3-(methylamino)azetidin-1-yl)phenol hydrochloride (520 mg, 94%) as a cream solid. LC-MS: m/z=235 [M+H]⁺.

### Step 3: 1-(tert-butyldimethylsilyl)-N-(1-(4-hydroxyphenyl)azetidin-3-yl)-N-methyl-1H-indole-6-sulfonamide

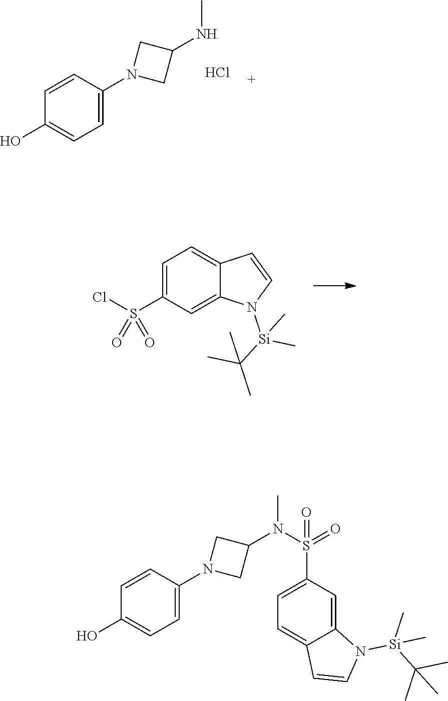 [SMILES: CNC1CN(c2ccc(O)cc2)C1 ; Cl ; CC(C)(C)[Si](C)(C)n1ccc2ccc(S(=O)(=O)Cl)cc21 ; CN(C1CN(c2ccc(O)cc2)C1)S(=O)(=O)c1ccc2ccn([Si](C)(C)C(C)(C)C)c2c1]

To a solution suspension of 4-(3-(methylamino)azetidin-1-yl)phenol hydrochloride (100 mg, 0.441 mmol) in DMF (5 mL) was added triethylamine (0.12 mL, 0.882 mmol) and the mixture was stirred at RT for 10 min. 2-Fluorobenzenesulfonyl chloride (0.059 mL, 0.441 mmol) was added dropwise and the reaction mixture was stirred for 1 h then quenched with water and diluted with DCM (5 mL). The layers were separated, and the organic layer was dried over anhydrous sulfate sodium and concentrated under reduced pressure. The residue (120 mg) was dissolved in acetonitrile (1 mL) and purified by preparative HPLC (10-80% acetonitrile (0.035% TFA) in water (0.05% TFA) eluent) yielding I-(tert-butyldimethylsilyl)-N-(1-(4-hydroxyphenyl)azetidin-3-yl)-N-methyl-1H-indole-6-sulfonamide (56 mg, 35%) as an beige powder Step 4: N-[1-(4-hydroxyphenyl)azetidin-3-yl]-N-methyl-1H-indole-6-sulfonamide

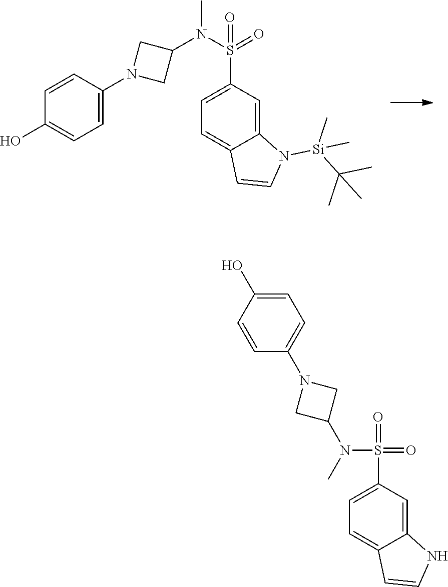 [SMILES: CN(C1CN(c2ccc(O)cc2)C1)S(=O)(=O)c1ccc2ccn([Si](C)(C)C(C)(C)C)c2c1 ; CN(C1CN(c2ccc(O)cc2)C1)S(=O)(=O)c1ccc2cc[nH]c2c1]

To a solution of 1-(tert-butyldimethylsilyl)-N-(1-(4-hydroxyphenyl)azetidin-3-yl)-N-methyl-1H-indole-6-sulfonamide (56 mg, 0.0332 mmol) in THF (330 μL) at RT was added a solution of tetrabutylammonium fluoride (1M in THF, 40 μL, 0.0399 mmol). The reaction mixture was stirred at RT for 1.5 h then diluted with water. The aqueous layer was extracted twice with EtOAc and the combined organic layers were washed with brine, dried over a phase separator, and concentrated under reduced pressure. The crude material was purified by flash chromatography (4 g silica gel, 0-50% acetone in DCM). The appropriate fractions were gathered and purified again by reverse-phase flash chromatography (C18 4 g, 0%-100% acetonitrile in water eluent). The appropriate fractions were gathered concentrated under reduced pressure. The resulting solid was taken-up with pentane and in diethyl ether to afford N-[1-(4-hydroxyphenyl)azetidin-3-yl]-N-methyl-1H-indole-6-sulfonamide (7.8 mg, 64%). LC-MS: m/z (NH)=358 [M+H]⁺. ¹H NMR (400 MHz, DMSO) δ 11.61 (s, 1H), 8.61 (s, 1H), 7.86 (s, 1H), 7.78 (d, J=8.4 Hz, 1H), 7.68 (t, J=2.8 Hz, 1H), 7.37 (dd, J=8.5, 1.7 Hz, 1H), 6.62 (s, 1H), 6.58 (d, J=8.8 Hz, 2H), 6.24 (d, J=8.7 Hz, 2H), 4.41 (t, J=6.5 Hz, 1H), 3.82 (t, J=7.6 Hz, 2H), 3.49 (t, J=6.8 Hz, 2H), 2.69 (s, 3H).

### Example 19: 4-[cyclopropyl-[1-(1H-indol-6-ylsulfonyl)azetidin-3-yl]amino]phenol

### Step 1: Tert-butyl 3-(4-tert-butoxyanilino)azetidine-1-carboxylate

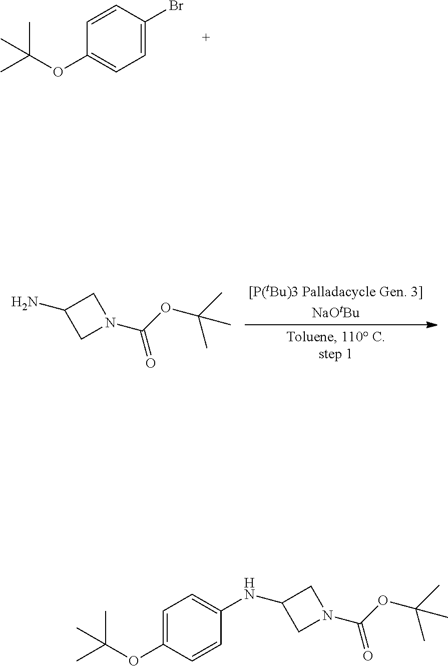 [SMILES: CC(C)(C)Oc1ccc(Br)cc1 ; CC(C)(C)OC(=O)N1CC(N)C1 ; CC(C)(C)OC(=O)N1CC(Nc2ccc(OC(C)(C)C)cc2)C1]

The title compound was synthesized from of tert-butyl 3-aminoazetidine-1-carboxylate (98%, 375 mg, 2.13 mmol) in a similar manner as Example 11 and purified through a silica gel column with 0-100% ethyl acetate in heptane to afford the title compound as a white powder (315 mg, 44%). LC-MS: m/z=320.0 [M+H]⁺.

### Step 2: Tert-butyl 3-(4-tert-butoxy-N-cyclopropyl-anilino)azetidine-1-carboxylate

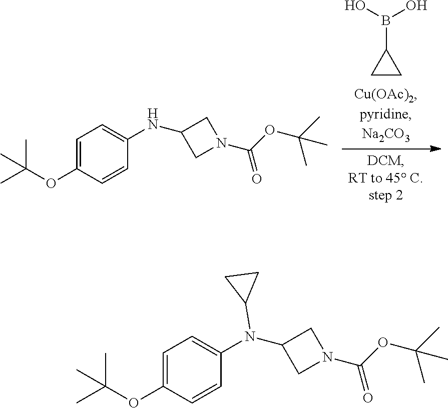

To a solution of tert-butyl 3-(4-tert-butoxyanilino)azetidine-1-carboxylate (315 mg, 0.983 mmol) in DCM (4 mL), sodium carbonate (313 mg, 2.95 mmol), pyridine (87 μL, 1.08 mmol), diacetoxycopper (196 mg, 1.08 mmol) and cyclopropylboronic acid (96%, 264 mg, 2.95 mmol) were added at room temperature. The reaction was stirred at rt and an opening air for 18 h. The reaction was then heated at 45° C. for 7 h and the reaction was stirred 48 h at rt. The mixture was diluted with DCM and washed with aqueous saturated NH₄Cl solution. The organic layer was dried over sodium sulfate and concentrated in vacuo. The crude material was purified through a silica gel column with 0-100% ethyl acetate in heptane to afford the title compound as a yellow solid (260 mg, 73%). LC-MS: m/z=361.1 [M+H]⁺.

### Step 3: 4-[azetidin-3-yl(cyclopropyl)amino]phenol (TFA Salt)

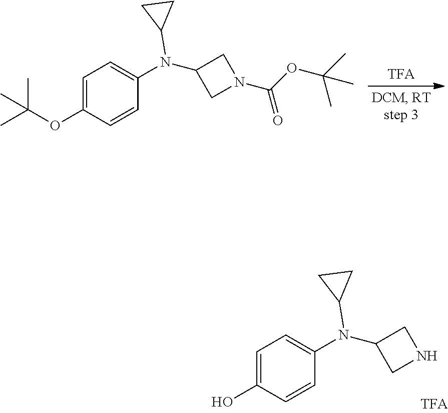 [SMILES: CC(C)(C)OC(=O)N1CC(N(c2ccc(OC(C)(C)C)cc2)C2CC2)C1 ; Oc1ccc(N(C2CC2)C2CNC2)cc1]

To a solution of tert-butyl 3-(4-tert-butoxy-N-cyclopropyl-anilino)azetidine-1-carboxylate (350 mg, 0.97 mmol) in DCM (4 mL), TFA (0.74 mL, 9.71 mmol) was added and the mixture was stirred at rt for 2 h until SM was consumed. The solvent was removed under reduced pressure. The crude compound was dissolved in MeOH and a resin of NaHCO₃ was added until pH 7. The mixture was filtered, and the filtrate was concentrated in vacuo to afford the title compound as a brown solid (175 mg, 40%). LC-MS: m/z=205.0 [M+H]⁺.

### Step 4: 4-[cyclopropyl-[1-(1H-indol-6-ylsulfonyl)azetidin-3-yl]amino]phenol

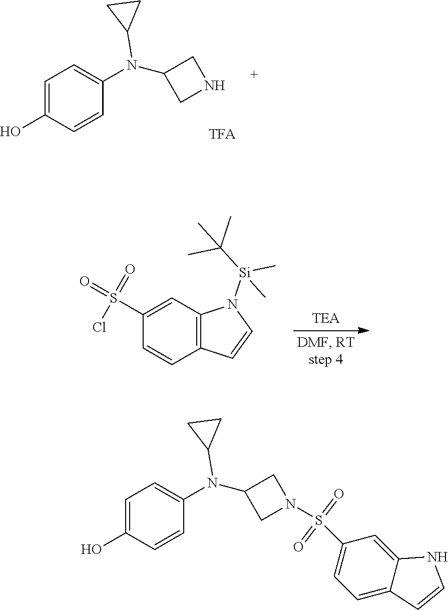 [SMILES: Oc1ccc(N(C2CC2)C2CNC2)cc1 ; CC(C)(C)[Si](C)(C)n1ccc2ccc(S(=O)(=O)Cl)cc21 ; O=S(=O)(c1ccc2cc[nH]c2c1)N1CC(N(c2ccc(O)cc2)C2CC2)C1]

To a stirred solution of 4-[azetidin-3-yl(cyclopropyl)amino]phenol (75 mg, 0.236 mmol) in DMF (1.2 mL), triethylamine (82 μL, 0.59 mmol) and 1-(tert-butyldimethylsilyl)-1H-indole-6-sulfonyl chloride (95%, 65 mg, 0.19 mmol) were added. The mixture was stirred at RT for 20 min. The mixture was diluted with EtOAc and washed with an aqueous saturated NaHCO₃ solution. The organic layer was dried over sodium sulfate and concentrated in vacuo. The crude material was purified through a silica gel column with 0-100% ethyl acetate in heptane and then lyophilized to afford 4-[cyclopropyl-[1-(1H-indol-6-ylsulfonyl)azetidin-3-yl]amino]phenol (44 mg, 47%). LC-MS: m/z=383.9 [M+H]⁺. ¹H NMR (400 MHz, DMSO) δ 11.63 (s, 1H), 8.87 (s, 1H), 7.88 (dt, J=1.6, 0.8 Hz, 1H), 7.81 (dd, J=8.4, 0.6 Hz, 1H), 7.75-7.65 (m, 1H), 7.40 (dd, J=8.3, 1.7 Hz, 1H), 6.64 (d, J=3.1 Hz, 1H), 6.62-6.50 (m, 4H), 4.12 (p, J=7.4 Hz, 1H), 3.87-3.74 (m, 2H), 3.69 (dd, J=8.3, 7.2 Hz, 2H), 1.82 (tt, J=6.7, 3.7 Hz, 1H), 0.24 (td, J=6.7, 4.6 Hz, 2H).

### Example 25: 4-[ethyl-[1-(1H-indol-6-ylsulfonyl)azetidin-3-yl]amino]phenol

### Step 1: Tert-butyl 3-(4-tert-butoxyanilino)azetidine-1-carboxylate

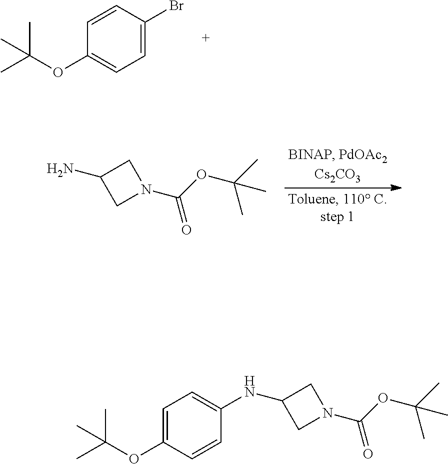 [SMILES: CC(C)(C)Oc1ccc(Br)cc1 ; CC(C)(C)OC(=O)N1CC(N)C1 ; CC(C)(C)OC(=O)N1CC(Nc2ccc(OC(C)(C)C)cc2)C1]

To a solution of tert-butyl 3-aminoazetidine-1-carboxylate (98%, 2.00 g, 11.4 mmol), 1-bromo-4-tert-butoxybenzene (97%, 2688 mg, 11.4 mmol), rac-BINAP (97%, 731 mg, 1.14 mmol) and cesium carbonate (9.76 g, 30.0 mmol) in anhydrous toluene (57 mL). The reaction mixture was degassed with argon for 5 min then Pd(OAc)₂ (258 mg, 1.14 mmol) was added and the reaction mixture was further degassed with argon for 5 min. Then the reaction mixture was heated-up to 110° C. for 18 h. The reaction was cooled down then diluted with EtOAc sat. NH₄Cl solution. The layers were separated and the organic layer was dried over sodium sulfate and concentrated in vacuo. The crude material was purified through a silica gel column with 0-100% ethyl acetate in heptane to afford the title compound (750 mg, 20%). LC-MS: m/z=265.1 [M+H]⁺.

### Step 2: Tert-butyl 3-(4-tert-butoxy-N-ethyl-anilino)azetidine-1-carboxylate

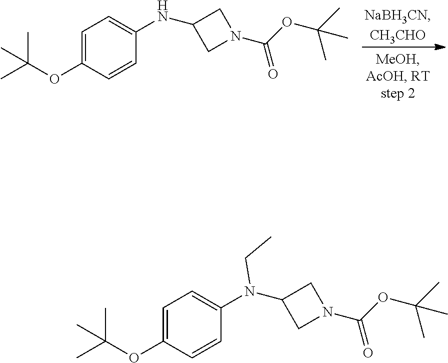

To a solution of tert-butyl 3-(4-tert-butoxyanilino)azetidine-1-carboxylate (100 mg, 0.312 mmol) in anhydrous DCM (1.6 mL), acetaldehyde (21 μL, 0.374 mmol) and acetic acid (0.14 mL) were added. The mixture was stirred at RT for 30 min. NaBH₃CN (312 mg, 0.624 mmol) was added, and the mixture was stirred at RT for 18 h. The reaction was quenched with sat. NaHCO₃ and the mixture was extracted with DCM. The organic layer was dried over sodium sulfate and concentrated in vacuo. The crude was used in the next step without further purification. (92 mg, 74%). LC-MS: m/z=349.5 [M+H]+

### Step 3: 4-[azetidin-3-yl(ethyl)amino]phenol (TFA salt)

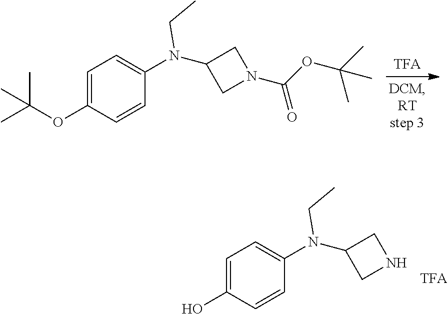 [SMILES: CCN(c1ccc(OC(C)(C)C)cc1)C1CN(C(=O)OC(C)(C)C)C1 ; CCN(c1ccc(O)cc1)C1CNC1]

The Boc deprotection was performed as described in Example 17 (step 2) using tert-butyl 3-(4-tert-butoxy-N-ethyl-anilino)azetidine-1-carboxylate (92 mg, 0.264 mmol) and was isolated as a pale oil (77 mg, 89%). LC-MS: m/z=381.1 [M+H]⁺.

### Step 4: 4-[ethyl-[1-(1H-indol-6-ylsulfonyl)azetidin-3-yl]amino]phenol

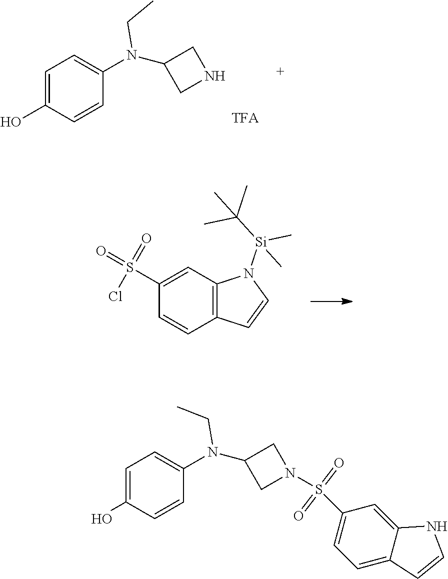 [SMILES: CCN(c1ccc(O)cc1)C1CNC1 ; CC(C)(C)[Si](C)(C)n1ccc2ccc(S(=O)(=O)Cl)cc21 ; CCN(c1ccc(O)cc1)C1CN(S(=O)(=O)c2ccc3cc[nH]c3c2)C1]

To a solution of 4-[azetidin-3-yl(ethyl)amino]phenol (TFA salt) (77 mg, 0.251 mmol) in DMF (1.3 mL), 1-(tert-butyldimethylsilyl)-1H-indole-6-sulfonyl chloride (70 mg, 0.201 mmol) and triethylamine (0.070 mL, 0.503 mmol) were added. The reaction mixture stirred for 45 min at rt. The reaction was stirred at room temperature for 2 h. The mixture was diluted with EtOAc and washed with an aqueous saturated NaHCO₃solution. The organic layer was dried over sodium sulfate and concentrated in vacuo. The crude material was purified through a silica gel column with 0-100% ethyl acetate in heptane to afford the title compound (45.1 mg, 47%). LC-MS: m/z=372.3 [M+H]⁺. ¹H NMR (400 MHz, DMSO) δ 11.63 (s, 1H), 8.88 (s, 1H), 7.86 (dt, J=1.6, 0.8 Hz, 1H), 7.80 (d, J=8.3 Hz, 1H), 7.70 (t, J=2.7 Hz, 1H), 7.39 (dd, J=8.3, 1.7 Hz, 1H), 6.66-6.60 (m, 1H), 6.59-6.52 (m, 2H), 6.52-6.45 (m, 21H), 3.90 (td, J=8.4, 6.3 Hz, 3H), 3.40-3.32 (m, 2H), 2.74-2.66 (m, 2H), 0.61 (t, J=7.0 Hz, 3H).

### Example 29: 4-[[1-(1H-indol-6-ylsulfonyl)azetidin-3-yl]methyl-methyl-amino]phenol Step 1: tert-butyl 3-[(4-tert-butoxyanilino)methyl]azetidine-1-carboxylate

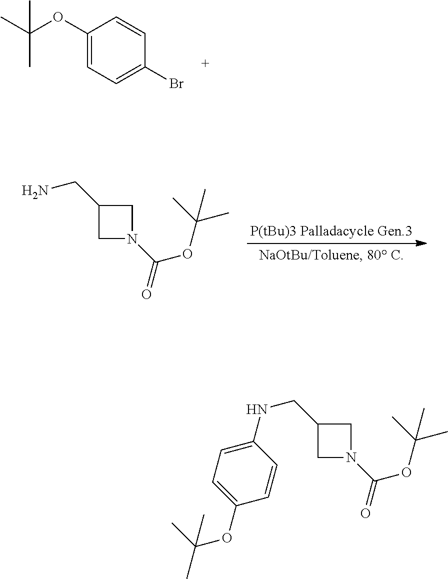 [SMILES: CC(C)(C)Oc1ccc(Br)cc1 ; CC(C)(C)OC(=O)N1CC(CN)C1 ; CC(C)(C)OC(=O)N1CC(CNc2ccc(OC(C)(C)C)cc2)C1]

A solution of tert-butyl 3-(methylamino)azetidine-1-carboxylate (1.00 g, 5.10 mmol), 1-bromo-4-tert-butoxybenzene (97%, 1205 mg, 5.10 mmol) and sodium tert-butoxide (1471 mg, 15.3 mmol) in toluene (25.5 mL) was degassed with argon for 10 min then [P(t-Bu)₃ Palladacycle Gen. 3] (98%, 149 mg, 0.26 mmol) was added and the mixture was heated-up to 110° C. for 2 h. The reaction was cooled down and diluted with EtOAc and sat. NH₄Cl solution. The organic layer was dried over sodium sulfate and concentrated under reduced pressure. The crude material was purified through a silica gel column with 0-100% ethyl acetate in heptane to afford the title compound as a brown oil (1.5 g, 84%). LC-MS: m/z=335 [M+H]⁺.

### Step 2: tert-butyl 3-[(4-tert-butoxy-N-methyl-anilino)methyl]azetidine-1-carboxylate

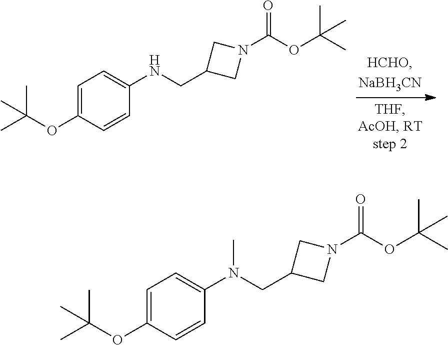

To a solution of tert-butyl 3-[(4-tert-butoxyanilino)methyl]azetidine-1-carboxylate (300 mg, 0.897 mmol) and formaldehyde (40 μL, 1.08 mmol) in acetic acid (900 μL) and THF (9 mL). The reaction mixture was stirred at RT for 30 min and sodium cyanoborohydride (polymer supported, 2 mmol/g, 897 mg, 1.79 mmol) was added. The reaction mixture was stirred at RT for 1 h. The reaction mixture was filtered and the resin was washed with THF. The filtrate was concentrated and taken up in DCM. Sat. NaHCO₃ was added and the layers were separated. The organic layer was dried over a phase separator and concentrated under reduced pressure to afford tert-butyl 3-[(4-tert-butoxy-N-methyl-anilino)methyl]azetidine-1-carboxylate (343 mg, Quant.) as an off-white powder. LC-MS: m/z=349 [M+H]⁺.

### Step 3: 4-[azetidin-3-ylmethyl(methyl)amino]phenol

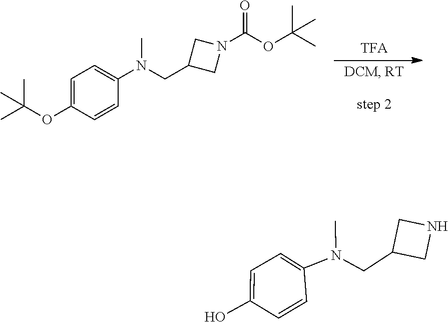 [SMILES: CN(CC1CN(C(=O)OC(C)(C)C)C1)c1ccc(OC(C)(C)C)cc1 ; CN(CC1CNC1)c1ccc(O)cc1]

The title compound was synthesized using the same procedure described in Example 17 (step 2) using tert-butyl 3-[(4-tert-butoxy-N-methyl-anilino)methyl]azetidine-1-carboxylate (282 mg, 0.728 mmol), and was isolated as an orange oil (133 mg, 95%) and was used in next step without more purification. LC-MS: m/z=193 [M+H]⁺.

### Step 4: 1:1 mixture of 4-[[1-[1-[tert-butyl(dimethyl)silyl]indol-6-yl]sulfonylazetidin-3-yl]methyl-methyl-amino]phenol and 4-[[1-(1H-indol-6-ylsulfonyl)azetidin-3-yl]methyl-methyl-amino]phenol

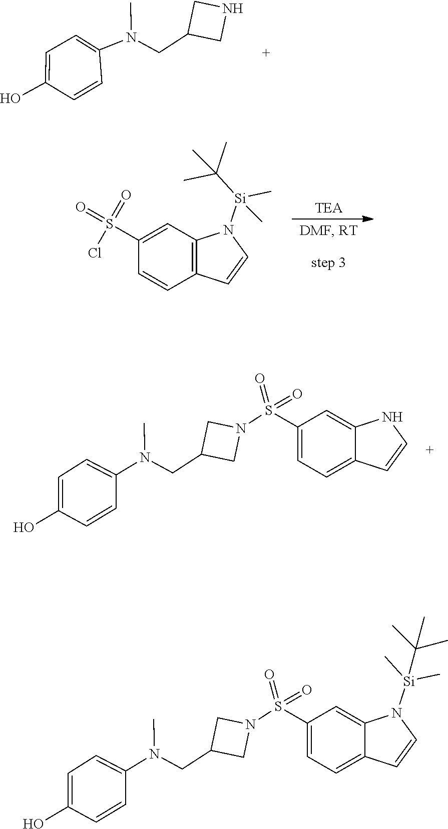 [SMILES: CN(CC1CNC1)c1ccc(O)cc1 ; CC(C)(C)[Si](C)(C)n1ccc2ccc(S(=O)(=O)Cl)cc21 ; CN(CC1CN(S(=O)(=O)c2ccc3cc[nH]c3c2)C1)c1ccc(O)cc1 ; CN(CC1CN(S(=O)(=O)c2ccc3ccn([Si](C)(C)C(C)(C)C)c3c2)C1)c1ccc(O)cc1]

The title compounds were synthesized as described in Example 16 (step 3), using 4-[azetidin-3-ylmethyl(methyl)amino]phenol (63 mg, 0.328 mmol) and 1-(tert-butyldimethylsilyl)-1H-indole-6-sulfonyl chloride (95%, 91 mg, 0.262 mmol), yielding a mixture 1:1 of desired product and deprotected analog as an brown powder used as is for the next step (101 mg, 50%). LC-MS: m/z=372 [M+H], Rt=0.74 min, m/z=486 [M+H]⁺.

### Step 5: 4-[[1-(1H-indol-6-ylsulfonyl)azetidin-3-yl]methyl-methyl-amino]phenol

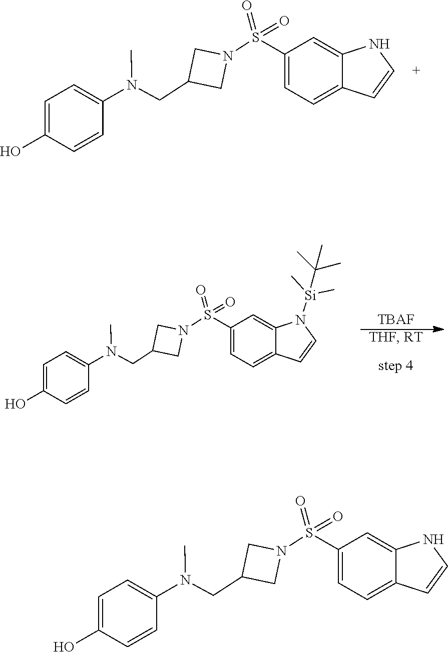

To a solution of a 1:1 mixture of 4-[[1-[1-[tert-butyl(dimethyl)silyl]indol-6-yl]sulfonylazetidin-3-yl]methyl-methyl-amino]phenol and 4-[[1-(1H-indol-6-ylsulfonyl)azetidin-3-yl]methyl-methyl-amino]phenol (101 mg, 0.0665 mmol) in anhydrous THF (0.6654 mL) was added a solution of tetrabutylammonium fluoride (1.0M in THF, 0.080 mL, 0.0799 mmol)). The reaction mixture was stirred at RT for 1 h then diluted with water. The aqueous layer was extracted twice with EtOAc and the combined organic layers were washed once with brine, dried over a phase separator, and concentrated under reduced pressure. The crude material was purified by flash chromatography (10 g silica gel, 0-100% EtOAc in cyclohexane), the appropriate fractions were gathered and purified again by preparative HPLC (0-100% acetonitrile in water). The desired fractions were gathered and concentrated under reduced pressure to afford 4-[[1-(1H-indol-6-ylsulfonyl)azetidin-3-yl]methyl-methyl-amino]phenol (31.6 mg, quant.) LC-MS: m/z=372 [M+H]⁺. ¹H NMR (400 MHz, DMSO) δ 11.66 (s, 1H), 8.61 (s, 1H), 7.90-7.79 (m, 2H), 7.71 (d, J=3.1 Hz, 1H), 7.41 (dd, J=8.4, 1.6 Hz, 1H), 6.66 (d, J=3.1 Hz, 1H), 6.55-6.46 (m, 2H), 6.37-6.28 (m, 2H), 3.76 (t, J=8.3 Hz, 2H), 3.36 (dd, J=8.3, 6.1 Hz, 2H), 2.82 (d, J=7.2 Hz, 2H), 2.63 (h, J=7.1 Hz, 1H), 2.47 (s, 3H).

### Example 35: 4-[5-(1H-indol-6-ylsulfonyl)-2,5-diazabicyclo[2.2.1]heptan-2-yl]phenol

### Step 1: Tert-butyl 5-(4-tert-butoxyphenyl)-2,5-diazabicyclo[2.2.1]heptane-2-carboxylate

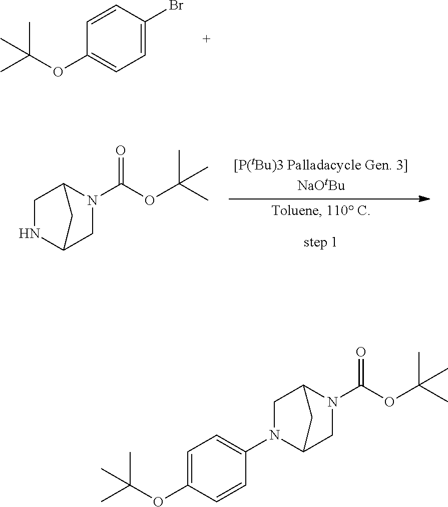 [SMILES: CC(C)(C)Oc1ccc(Br)cc1 ; CC(C)(C)OC(=O)N1CC2CC1CN2 ; CC(C)(C)OC(=O)N1CC2CC1CN2c1ccc(OC(C)(C)C)cc1]

A solution of tert-butyl 2,5-diazabicyclo[2.2.1]heptane-2-carboxylate (150 mg, 0.757 mmol), sodium tert-butoxide (218 mg, 2.27 mmol), 1-bromo-4-tert-butoxybenzene (97%, 197 mg, 0.832 mmol) in toluene (3.8 mL) was degassed with argon for 10 min then P(t-Bu)₃ Pd G3 (98%, 44 mg, 0.08 mmol) was added and the reaction mixture was heated at 110° C. with stirring for 3 h. The reaction was cooled down to RT and diluted with EtOAc sat. NH₄Cl solution. The layers were separated and the organic layer was dried over sodium sulfate and concentrated in vacuo. The crude material was purified through a silica gel column with 0-100% ethyl acetate in heptane to afford the title compound as a yellow powder (230 mg, 79%). LC-MS: m/z=347.4 [M+H]⁺

### Step 2: 4-(2,5-diazabicyclo[2.2.1]heptan-2-yl)phenol (TFA Salt)

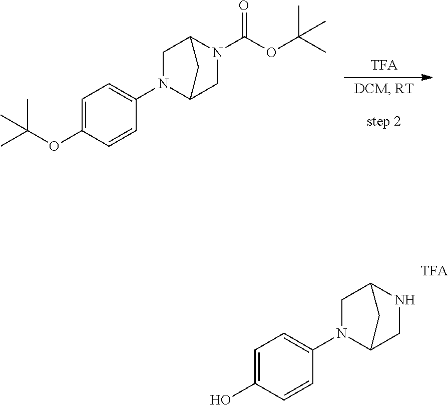 [SMILES: CC(C)(C)OC(=O)N1CC2CC1CN2c1ccc(OC(C)(C)C)cc1 ; Oc1ccc(N2CC3CC2CN3)cc1]

To a solution of tert-butyl 5-(4-tert-butoxyphenyl)-2,5-diazabicyclo[2.2.1]heptane-2-carboxylate (230 mg, 0.664 mmol) in DCM (3.3 mL), TFA (0.25 mL, 3.32 mmol) was added at room temperature. After 3 h of stirring, an excess of TFA was added and the reaction was stirred for 1 h. The solvent was removed in vacuo. The crude compound was dissolved in MeOH and a resin of NaHCO₃ was added until pH 7. The mixture was filtered, and the filtrate was concentrated in vacuo to afford the title compound as a beige powder. (160 mg, 75%). LC-MS: m/z=191.1 [M+H]⁺

### Step 3: 4-[5-(1H-indol-6-ylsulfonyl)-2,5-diazabicyclo[2.2.1]heptan-2-yl]phenol

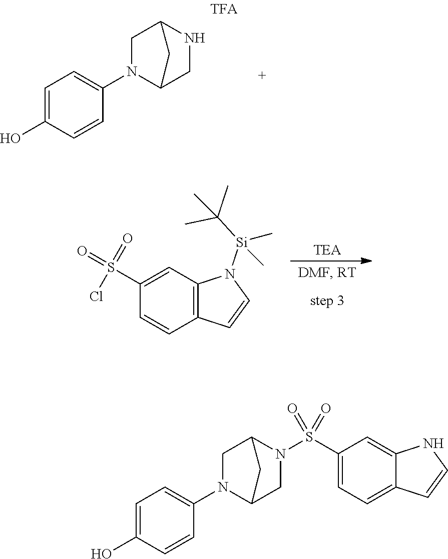 [SMILES: Oc1ccc(N2CC3CC2CN3)cc1 ; CC(C)(C)[Si](C)(C)n1ccc2ccc(S(=O)(=O)Cl)cc21 ; O=S(=O)(c1ccc2cc[nH]c2c1)N1CC2CC1CN2c1ccc(O)cc1]

The title compound was synthesized as described in Example 16 (step 3) using 4-(2,5-diazabicyclo[2.2.1]heptan-2-yl)phenol (50 mg, 0.164 mmol) and 1-(tert-butyldimethylsilyl)-1H-indole-6-sulfonyl chloride (95%, 51 mg, 0.148 mmol), and was isolated as a white solid (14 mg, 22%) after purification by flash chromatography (silica gel, 0-100%% EtOAc in heptane eluent) and lyophilization. LC-MS: m/z=369.8 [M+H]⁺. ¹H NMR (400 MHz, DMSO) δ 11.60 (s, 1H), 8.54 (s, 1H), 7.86 (dt, J=1.6, 0.8 Hz, 1H), 7.75 (dd, J=8.4, 0.6 Hz, 1H), 7.66 (d, J=3.1 Hz, 1H), 7.42 (dd, J=8.4, 1.7 Hz, 1H), 6.64-6.51 (m, 3H), 6.46-6.31 (m, 2H), 4.44 (s, 1H), 4.24 (s, 1H), 3.43 (dd, J=9.0, 2.4 Hz, 1H), 3.27 (dd, J=9.5, 1.1 Hz, 1H), 3.09 (dd, J=9.5, 1.9 Hz, 1H), 2.95 (d, J=8.9 Hz, 1H), 1.63 (dd, J=9.7, 2.0 Hz, 1H), 0.89-0.79 (m, 1H).

### Example 36: rac-4-(((2R,3R)-1-((1H-indol-6-yl)sulfonyl)-2-methylazetidin-3-yl)amino)phenol

The title compound was synthesized in a similar manner to Example 35 starting with tert-butyl (2R,3R)-3-amino-2-methylazetidine-1-carboxylate and yielding rac-4-(((2R,3R)-1-((1H-indol-6-yl)sulfonyl)-2-methylazetidin-3-yl)amino)phenol as a white powder. LC-MS: m/z=358 [M+H]⁺. 1H NMR (400 MHz, DMSO) δ 11.64 (s, 1H), 8.40 (s, 1H), 7.89 (d, J=1.4 Hz, 1H), 7.82 (d, J=8.3 Hz, 1H), 7.70 (d, J=3.1 Hz, 1H), 7.47-7.34 (m, 1H), 6.67-6.61 (m, 1H), 6.51-6.24 (m, 4H), 5.70 (d, J=8.2 Hz, 1H), 4.07 (p, J=6.6 Hz, 1H), 3.83 (t, J=8.4 Hz, 1H), 3.72 (qd, J=8.1, 3.7 Hz, 1H), 3.54 (dd, J=8.7, 3.7 Hz, 1H), 1.15 (d, J=6.5 Hz)

### Example 40: 4-[1-(1H-indol-6-ylsulfonyl)pyrrolidin-3-yl]oxyphenol

### Step 1: tert-butyl 3-(4-methoxyphenoxy)pyrrolidine-1-carboxylate

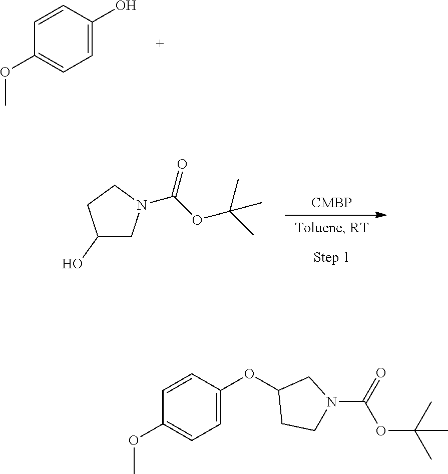

To a mixture of 4-methoxyphenol (585 mg, 4.71 mmol) and tert-butyl 3-hydroxypyrrolidine-1-carboxylate (98%, 300 mg, 1.57 mmol) in anhydrous toluene (7 mL) was added cyanomethylenetributylphosphorane (95%, 0.87 mL, 3.14 mmol) under argon at 25° C. The reaction mixture was stirred at 80° C. for 18 h. The reaction mixture was allowed to cool to RT and solvent was evaporated. The crude material was purified through a silica gel column with 0-60% ethyl acetate in heptane to afford tert-butyl 3-(4-methoxyphenoxy)pyrrolidine-1-carboxylate as a yellow oil (309 mg, 67%). LC-MS: m/z=238.2 [M-tBu]⁺.

### Step 2: 4-[1-(1H-indol-6-ylsulfonyl)pyrrolidin-3-yl]oxyphenol (HCl Salt)

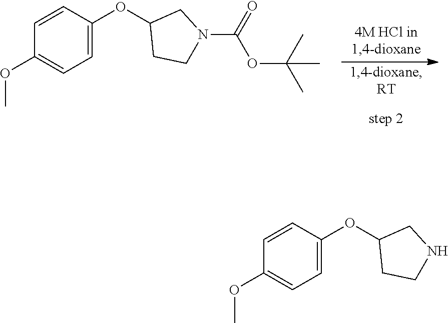 [SMILES: COc1ccc(OC2CCN(C(=O)OC(C)(C)C)C2)cc1 ; COc1ccc(OC2CCNC2)cc1]

To a solution of tert-butyl 3-(4-methoxyphenoxy)pyrrolidine-1-carboxylate (90/a, 143 mg, 0.44 mmol) in anhydrous dioxane (3 mL) was added a solution of HCl (4.0M in 1,4-dioxane, 1.1 mL, 4.39 mmol). The reaction mixture was stirred at RT for 18 h. The reaction mixture was filtered and the residue was washed with 1,4-dioxane and dried under reduced pressure to afford 3-(4-methoxyphenoxy)pyrrolidine as a beige powder which was used in next step without further purification. (102 mg, 97%). LC-MS: m/z=194.2 [M+H]⁺.

### Step 3: 4-pyrrolidin-3-yloxyphenol

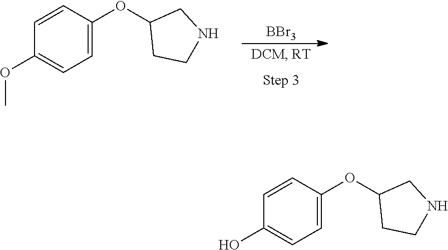

To a solution of 3-(4-methoxyphenoxy)pyrrolidine hydrochloride (26 mg, 0.12 mmol) in anhydrous DCM (0.7 mL) was added a solution of BBᵣ(1.0M in DCM, 0.23 mL, 0.23 mmol). The solution was stirred at RT for 4 h, then quenched with water. The aqueous layer was extracted with DCM (3×5 mL) and then a 1:2 mixture of isopropanol:chloroform (3×5 mL). The organic layer was dried using a phase separator and concentrated under reduced pressure to afford 4-pyrrolidin-3-yloxyphenol as a white powder which was used without further purification (20 mg, 96%). LC-MS: m/z=180.2 [M+H]⁺.

### Step 4: 4-[1-(1-triisopropylsilylindol-6-yl)sulfonylpyrrolidin-3-yl]oxyphenol

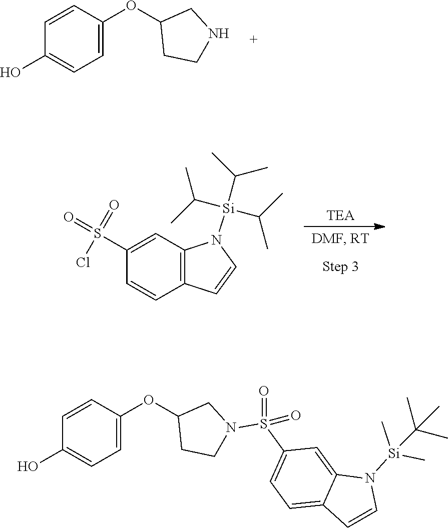 [SMILES: Oc1ccc(OC2CCNC2)cc1 ; CC(C)[Si](C(C)C)(C(C)C)n1ccc2ccc(S(=O)(=O)Cl)cc21 ; CC(C)(C)[Si](C)(C)n1ccc2ccc(S(=O)(=O)N3CCC(Oc4ccc(O)cc4)C3)cc21]

The title compound was synthesized as described in Example 16 (step 3) using 4-pyrrolidin-3-yloxyphenol (27 mg, 0.15 mmol) and 1-triisopropylsilylindole-6-sulfonyl chloride (50 mg, 0.14 mmol), yielding 4-[1-(1-triisopropylsilylindol-6-yl)sulfonylpyrrolidin-3-yl]oxyphenol as a yellow oil which was used without further purification (48 mg, 50%). LC-MS: m/z=515.3 [M+H]⁺.

### Step 5: 4-[1-(1H-indol-6-ylsulfonyl)pyrrolidin-3-yl]oxyphenol

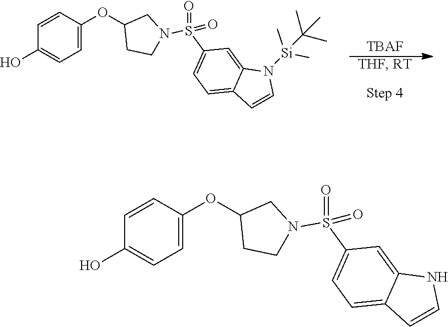

To a solution of 4-[1-(1-triisopropylsilylindol-6-yl)sulfonylpyrrolidin-3-yl]oxyphenyl (48 mg, 0.093 mmol) in anhydrous THF (0.5 mL) was added a solution of TBAF (1.0M in THF, 0.10 mL, 0.11 mmol). The reaction mixture was stirred at room temperature for 30 min. Water was added and the aqueous layer was extracted with EtOAc (3×5 mL). The combined organic layers were washed once with brine, dried using a phase separator and concentrated under reduced pressure. The crude material was purified through a silica gel column with 0-5% MeOH in DCM and then lyophilized to afford 4-[1-(1H-indol-6-ylsulfonyl)pyrrolidin-3-yl]oxyphenol (12.9 mg, 39%). LC-MS: m/z=359.3 [M+H]⁺. 1H NMR (400 MHz, DMSO) δ 11.58 (s, 1H), 8.90 (s, 1H), 7.85 (d, J=1.5 Hz, 1H), 7.75 (d, J=8.3 Hz, 1H), 7.67 (d, J=3.0 Hz, 1H), 7.39 (dd, J=8.4, 1.6 Hz, 1H), 6.62 (d, J=3.0 Hz, 1H), 6.56-6.45 (m, 2H), 6.42-6.33 (m, 2H), 4.73 (s, 1H), 3.37 (dd, J=11.4, 4.6 Hz, 1H), 3.34-3.27 (m, 1H), 3.27-3.09 (m, 2H), 1.93 (ddd, J=14.7, 11.8, 7.5 Hz, 2H)

### Example 41: 4-(2-((1-methyl-1H-indol-6-yl)sulfonyl)-2,8-diazaspiro[4.5]decan-8-yl)phenol

The title compounds was made according to Example 17 using 1-methyl-1H-indole-6-sulfonyl chloride. LC-MS: m/z=426 [M+H]+, 11H NMR (400 MHz, DMSO) δ 8.76 (s, 1H), 7.97-7.92 (m, 1H), 7.77 (d, J=8.2 Hz, 1H), 7.63 (d, J=3.0 Hz, 1H), 7.46 (dd, J=8.3, 1.6 Hz, 1H), 6.74-6.66 (m, 2H), 6.64-6.56 (m, 31H), 3.91 (s, 3H), 3.23 (t, J=7.1 Hz, 2H), 3.11 (s, 2H), 2.84-2.67 (m, 4H), 1.59 (t, J=7.0 Hz, 2H), 1.32 (ddd, J=12.3, 8.2, 3.8 Hz, 2H), 1.23 (d, J=6.7 Hz, 2H).

### Example 43: 4-[[1-(1H-indol-6-ylsulfonyl)-4-piperidyl]oxy]phenol

### Step 1: tert-butyl 4-(4-methoxyphenoxy)piperidine-1-carboxylate

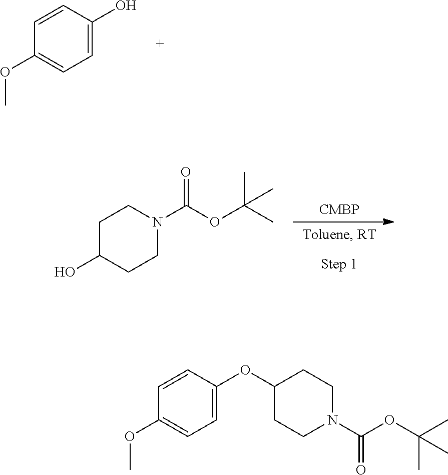

To a solution of 4-methoxyphenol (98%, 549 mg, 4.34 mmol) and tert-butyl 4-hydroxypiperidine-1-carboxylate (97%, 300 mg, 1.45 mmol) in anhydrous toluene (5.8 mL) was added (cyanomethylene) tributylphosphorane (98%, 0.77 mL, 2.89 mmol). The reaction mixture was purged with argon for 5 min and stirred at 80° C. for 18 h. The reaction mixture was allowed to cool to RT and the solvent was concentrated under reduced pressure. The crude material was purified through a silica gel column with 0-100% ethyl acetate in heptane to afford tert-butyl 4-(4-methoxyphenoxy)piperidine-1-carboxylate as a yellow oil (256 mg, 57%). LC-MS: m/z=252.2 [M+tBu]⁺.

### Step 2: 4-(4-methoxyphenoxy)piperidine

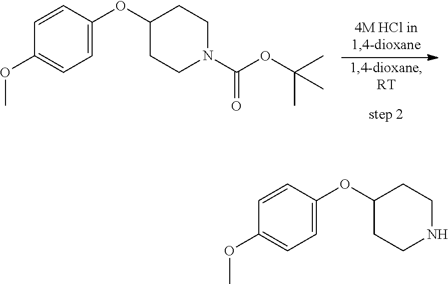 [SMILES: COc1ccc(OC2CCN(C(=O)OC(C)(C)C)CC2)cc1 ; COc1ccc(OC2CCNCC2)cc1]

To a solution of tert-butyl 4-(4-methoxyphenoxy)piperidine-1-carboxylate (251 mg, 0.82 mmol) in anhydrous dioxane (5.4 mL) was added a solution of hydrochloric acid (4.0M in 1,4-dioxane, 2.0 mL, 8.2 mmol) and the reaction mixture was stirred at RT for 18 h. The reaction mixture was filtered and the residue was rinsed with dioxane and dried under reduced pressure to afford 4-(4-methoxyphenoxy)piperidine hydrochloride as a beige powder (135 mg, 67%). LC-MS: m/z=208.3 [M+H]⁺.

### Step 3: 4-(4-piperidyloxy)phenol

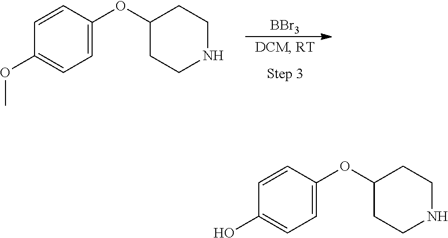

To a solution of 4-(4-methoxyphenoxy)piperidine hydrochloride (50 mg, 0.21 mmol) in anhydrous DCM (1.24 mL) was added a solution of BBr₃ (1.10M in DCM, 0.41 mL, 0.41 mmol). The solution was stirred at RT for 4 h then quenched with water and extracted with DCM (3×5 mL) followed by a mixture of isopropanol/chloroform (1/2) (3×5 mL). The organic layer was dried over a phase separator and concentrated under reduced pressure to afford 4-(4-piperidyloxy)phenol as a white powder (39 mg, 99%). LC-MS: m/z=194.2 [M+H]⁺.

### Step 4: 4-[[1-(1-triisopropylsilylindol-6-yl)sulfonyl-4-piperidyl]oxy]phenol

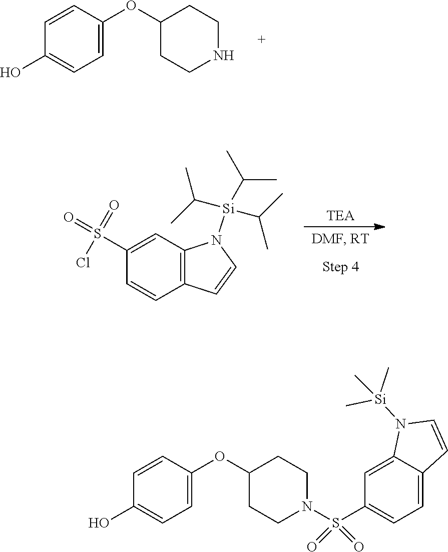 [SMILES: Oc1ccc(OC2CCNCC2)cc1 ; CC(C)[Si](C(C)C)(C(C)C)n1ccc2ccc(S(=O)(=O)Cl)cc21 ; C[Si](C)(C)n1ccc2ccc(S(=O)(=O)N3CCC(Oc4ccc(O)cc4)CC3)cc21]

The title compound was synthesized as described in Example 16 (step 3) using with 4-(4-piperidyloxy)phenol (39 mg, 0.21 mmol) and 1-triisopropylsilylindole-6-sulfonyl chloride (68 mg, 0.18 mmol), and isolated as a yellow oil (46 mg, 22%). LC-MS: m/z=529.3 [M+H]⁺.

### Step 5: 4-[[1-(1H-indol-6-ylsulfonyl)-4-piperidyl]oxy]phenol

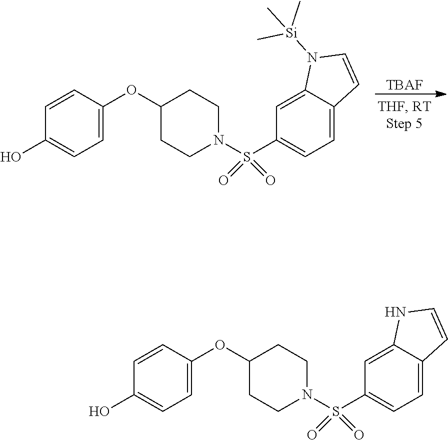

To a solution of 4-[[1-(1H-indol-6-ylsulfonyl)-4-piperidyl]oxy]phenol (10.4 mg, 64.2% Yield) in anhydrous THF (0.23 mL) was added TBAF (solution 1M in THF, 0.05 mL, 0.05 mmol). The reaction mixture was stirred at room temperature for 30 min. Water was added and the aqueous layer was extracted with EtOAc (3×5 mL). The combined organic layers were washed once with brine, dried over a phase separator, and concentrated under reduced pressure. The crude material was purified through a silica gel column with 0-10% MeOH in DCM and then lyophilized to afford 4-[[1-(1H-indol-6-ylsulfonyl)-4-piperidyl]oxy]phenol (10.4 mg, 65%). LC-MS: m/z=373.4 [M+H]⁺. ¹H NMR (400 MHz, DMSO) δ 11.62 (s, 1H), 8.88 (s, 1H), 7.97-7.73 (m, 2H), 7.68 (d, J=3.1 Hz, 1H), 7.35 (dd, J=8.4, 1.7 Hz, 11-1), 6.79-6.19 (m, 5H), 4.16 (dt, J=7.8, 4.1 Hz, 1H), 3.28-3.20 (m, 2H), 2.83-2.72 (m, 2H), 1.97-1.87 (m, 2H), 1.62 (dtd, J=12.4, 8.3, 3.6 Hz, 2H).

### Example 44: 4-(4-hydroxyanilino)-1-(1H-indol-6-ylsulfonyl)pyrrolidin-3-ol

The title compound was prepared according to Example 17 starting from tert-Butyl 3-amino-4-hydroxypyrrolidine-1-carboxylate yielding 4-(4-hydroxyanilino)-1-(1H-indol-6-ylsulfonyl)pyrrolidin-3-ol (37 mg, 57%) after purification by flash chromatography (silica gel column, 0-10% methanol in DCM). LC-MS: m/z=374.3 [M+H]⁺. ¹H NMR (400 MHz, DMSO) δ 11.58 (s, 1H), 8.43 (s, 1H), 7.88-7.84 (m, 1H), 7.74 (d, J=8.3 Hz, 1H), 7.66 (dd, J=3.0, 2.0 Hz, 1H), 7.39 (dd, J=8.4, 1.6 Hz, 1H), 6.61 (d, J=3.0 Hz, 1H), 6.54-6.46 (m, 2H), 6.37-6.28 (m, 2H), 5.14 (d, J=3.8 Hz, 1H), 4.82 (d, J=5.8 Hz, 1M), 3.91 (tt, J=5.0, 2.6 Hz, 1H), 3.49-3.39 (m, 3H), 3.09-2.98 (m, 2H).

### Example 45: 4-[[1-(1H-indol-6-ylsulfonyl)azetidin-2-yl]methyl-methyl-amino]phenol

### Step 1: tert-butyl 2-[(4-tert-butoxyanilino)methyl]azetidine-1-carboxylate

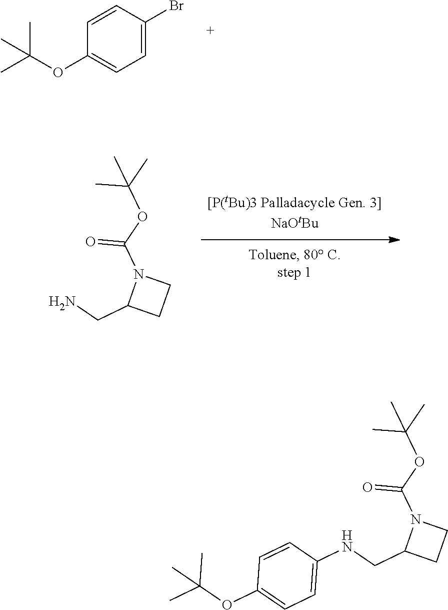 [SMILES: CC(C)(C)Oc1ccc(Br)cc1 ; CC(C)(C)OC(=O)N1CCC1CN ; CC(C)(C)OC(=O)N1CCC1CNc1ccc(OC(C)(C)C)cc1]

The title compound was synthesized according to Example 29 (1^{st} step) using tert-butyl 2-(aminomethyl) azetidine-1-carboxylate (600 mg, 3.06 mmol), and was isolated as a light yellow oil (470 mg, 46%) after purification by flash chromatography (25 g silica gel column, 0-15% EtOAc in heptane). LC-MS: m/z=335 [M+H]⁺.

### Step 2: tert-butyl 2-[(4-tert-butoxy-N-methyl-anilino)methyl]azetidine-1-carboxylate

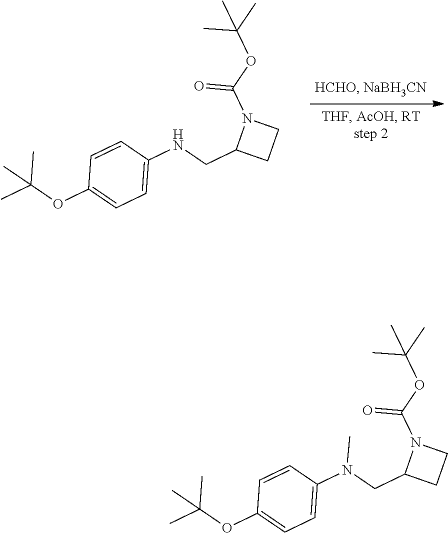 [SMILES: CC(C)(C)OC(=O)N1CCC1CNc1ccc(OC(C)(C)C)cc1 ; CN(CC1CCN1C(=O)OC(C)(C)C)c1ccc(OC(C)(C)C)cc1]

To a solution of tert-butyl 2-[(4-tert-butoxyanilino)methyl]azetidine-1-carboxylate (180 mg, 0.538 mmol) and formaldehyde (80 μL, 1.08 mmol) in acetic acid (292 μL) and THF (2.9 mL). The reaction mixture was stirred at RT for 30 min and sodium cyanoborohydride (polymer supported, 2 mmol/g, 673 mg, 1.35 mmol) was added. The reaction mixture was stirred at RT for 1 h. The reaction mixture was filtered and the resin was washed with THF. The filtrate was concentrated and taken up in DCM. Sat. NaHCO₃ was added and the layers were separated. The organic layer was dried over a phase separator and concentrated under reduced pressure to tert-butyl 2-[(4-tert-butoxy-N-methyl-anilino)methyl]azetidine-1-carboxylate (216 mg, Quant.) as a colorless oil. LC-MS: m/z=349 [M+H]⁺.

### Step 3: 4-[azetidin-2-ylmethyl(methyl)amino]phenol (TFA salt)

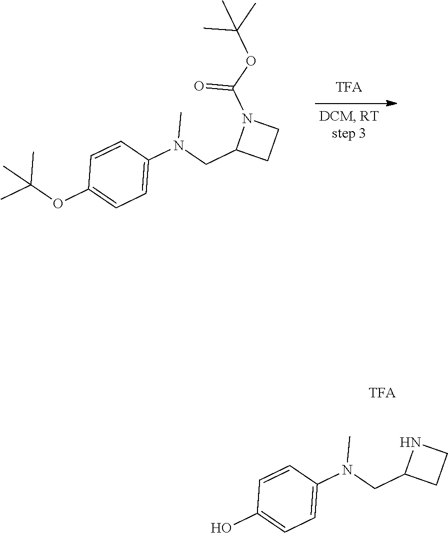 [SMILES: CN(CC1CCN1C(=O)OC(C)(C)C)c1ccc(OC(C)(C)C)cc1 ; CN(CC1CCN1)c1ccc(O)cc1]

To a solution of tert-butyl 2-[(4-tert-butoxy-N-methyl-anilino)methyl]azetidine-1-carboxylate (200 mg, 0.568 mmol) in 5 ml DCM was added 2.5 ml of TFA. The reaction was stirred 30 min at RT and the solvent was evaporated yielding 4-[azetidin-2-ylmethyl(methyl)amino]phenol (TFA salt) as a brown oil (49 mg, 23%) and was used in next step without more purification. LC-MS: m/z=193 [M+H]⁺.

### Step 4: 4-[[1-(1H-indol-6-ylsulfonyl)azetidin-2-yl]methyl-methyl-amino]phenol

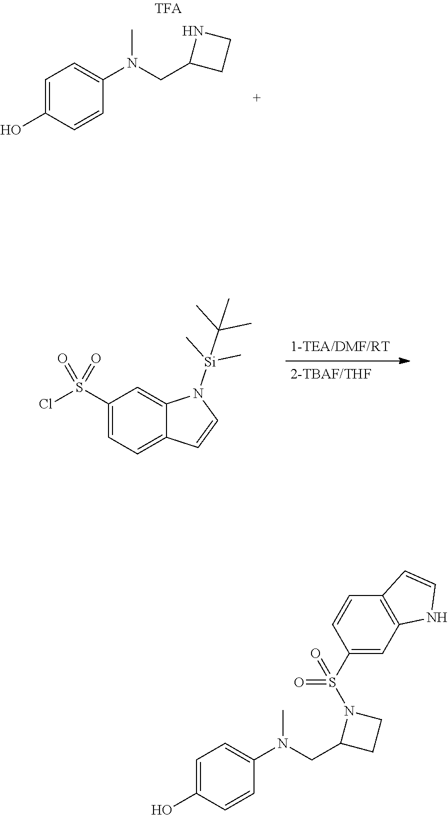 [SMILES: CN(CC1CCN1)c1ccc(O)cc1 ; CC(C)(C)[Si](C)(C)n1ccc2ccc(S(=O)(=O)Cl)cc21 ; CN(CC1CCN1S(=O)(=O)c1ccc2cc[nH]c2c1)c1ccc(O)cc1]

The title compounds were synthesized as described in Example 16 (step 3 and 4) using 4-[azetidin-2-ylmethyl(methyl)amino]phenol (TFA salt) (50 mg, 0.257 mmol) 1-triisopropylsilylindole-6-sulfonyl chloride (57 mg, 0.154 mmol), and was isolated (24 mg, 24%) after purification by flash chromatography (10 g silica gel, 0-2% MeOH in DCM). LC-MS: m/z=372 [M+H]⁺. ¹H NMR (400 MHz, DMSO) δ 11.65 (s, 1H), 8.60 (s, 1H), 7.90-7.85 (m, 1H), 7.76 (d, J=8.4 Hz, 1H), 7.72-7.66 (m, 1H), 7.34 (dd, J=8.3, 1.6 Hz, 1H), 6.71-6.56 (m, 5H), 3.83 (qd, J=7.8, 3.0 Hz, 1H), 3.65-3.56 (m, 2H), 3.40 (ddt, J=13.2, 8.7, 4.0 Hz, 2H), 2.81 (s, 3H), 2.11-1.97 (m, 1H), 1.88-1.74 (m, 1H).

### Example 47: 4-[2-(1H-indol-2-ylsulfonyl)-2,8-diazaspiro[4.5]decan-8-yl]phenol Intermediate: tert-butyl 2-[[8-(4-hydroxyphenyl)-2,8-diazaspiro[4.5]decan-2-yl]sulfonyl]indole-1-carboxylate

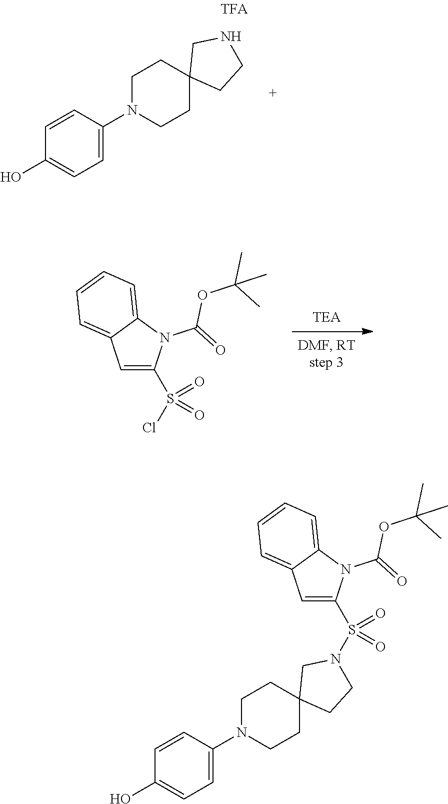 [SMILES: Oc1ccc(N2CCC3(CCNC3)CC2)cc1 ; CC(C)(C)OC(=O)n1c(S(=O)(=O)Cl)cc2ccccc21 ; CC(C)(C)OC(=O)n1c(S(=O)(=O)N2CCC3(CCN(c4ccc(O)cc4)CC3)C2)cc2ccccc21]

The N-boc protected intermediate was synthesized from 4-(2,8-diazaspiro [4.5] decan-8-yl) phenol (TFA salt) as described in Example 17 using tert-butyl 2-(chlorosulfonyl)-1H-indole-1-carboxylate yielding tert-butyl 2-((8-(4-hydroxyphenyl)-2,8-diazaspiro[4.5]decan-2-yl)sulfonyl)-1H-indole-1-carboxylate as an orange oil (129.5 mg, 100%). LCMS: m/z=512 [M+H]⁺. 4-[2-(1H-indol-2-ylsulfonyl)-2,8-diazaspiro[4.5]decan-8-yl]phenol

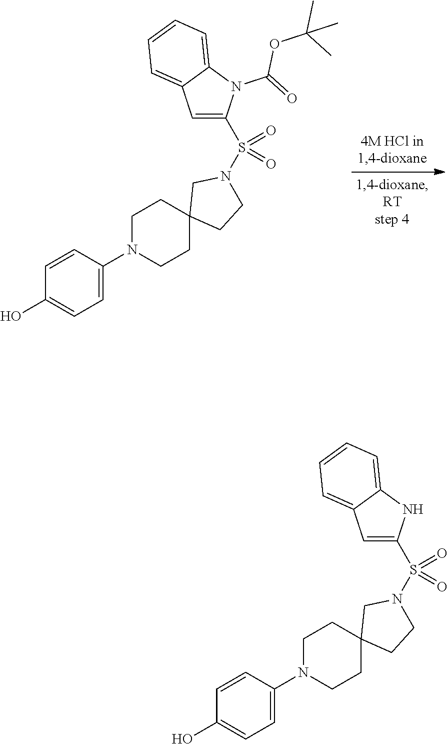 [SMILES: CC(C)(C)OC(=O)n1c(S(=O)(=O)N2CCC3(CCN(c4ccc(O)cc4)CC3)C2)cc2ccccc21 ; O=S(=O)(c1cc2ccccc2[nH]1)N1CCC2(CCN(c3ccc(O)cc3)CC2)C1]

To a solution of tert-butyl 2-[[8-(4-hydroxyphenyl)-2,8-diazaspiro[4.5]decan-2-yl]sulfonyl]indole-1-carboxylate (129 mg, 0.253 mmol) in anhydrous 1,4-dioxane (0.8 mL), was added dropwise a solution of HCl (4M in 1,4-dioxane, 0.51 mL, 2.02 mmol). Extra HCl (4M in 1,4-dioxane, 0.46 mL, 1.84 mmol). The reaction mixture was stirred at RT for 23 h then EtOAc and saturated NaHCO₃ were added and the layers were separated. The aqueous layer was extracted with EtOAc and the combined organic layers were dried over sodium sulfate, filtered, and concentrated under reduced pressure. The residue was triturated in pentane then dried under reduced pressure at 35° C. for 24 h to afford 4-[2-(1H-indol-2-ylsulfonyl)-2,8-diazaspiro[4.5]decan-8-yl]phenol (51 mg, 47%,). LCMS: m/z=412 [M+H]⁺. ¹H NMR (400 MHz, DMSO) δ 12.20 (s, 1H), 8.78 (s, 1H), 7.70 (dt, J=8.0, 1.0 Hz, 1H), 7.48 (dq, J=8.5, 1.0 Hz, 1H), 7.31 (ddd, J=8.2, 7.0, 1.2 Hz, 1H), 7.14 (ddd, J=8.0, 7.0, 1.0 Hz, 1H), 7.06 (d, J=0.9 Hz, 1H), 6.73-6.66 (m, 2H), 6.64-6.57 (m, 2H), 3.30 (s, 2H), 3.21 (s, 2H), 2.85-2.63 (m, 4H), 1.63 (t, J=7.1 Hz, 2H), 1.38-1.14 (m, 4H).

### Example 50: 4-[[(3S)-1-(1H-indol-6-ylsulfonyl)pyrrolidin-3-yl]-(3-pyridylmethyl)amino]phenol

### Step 1: tert-butyl (3S)-3-(4-tert-butoxyanilino)pyrrolidine-1-carboxylate

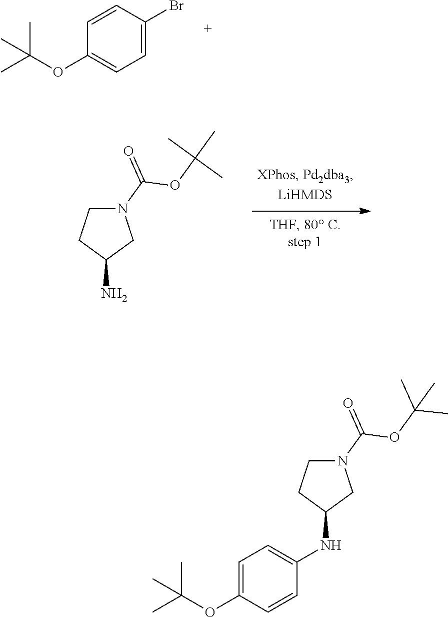 [SMILES: CC(C)(C)Oc1ccc(Br)cc1 ; CC(C)(C)OC(=O)N1CC[C@H](N)C1 ; CC(C)(C)OC(=O)N1CC[C@H](Nc2ccc(OC(C)(C)C)cc2)C1]

The title compound was synthesized as described for Example 30 (1^{st} step) (Buchwald reaction 1), using tert-butyl (3S)-3-aminopyrrolidine-1-carboxylate (97%, 1000 mg, 5.21 mmol), and was isolated as an orange powder (437 mg, 20%) after purification by flash chromatography (40 g silica gel column, 0-30% EtOAc in heptane). LC-MS: m/z=279 [M+H]⁺.

### Step 2: tert-butyl (3S)-3-[4-tert-butoxy-N-(3-pyridylmethyl)anilino]pyrrolidine-1-carboxylate

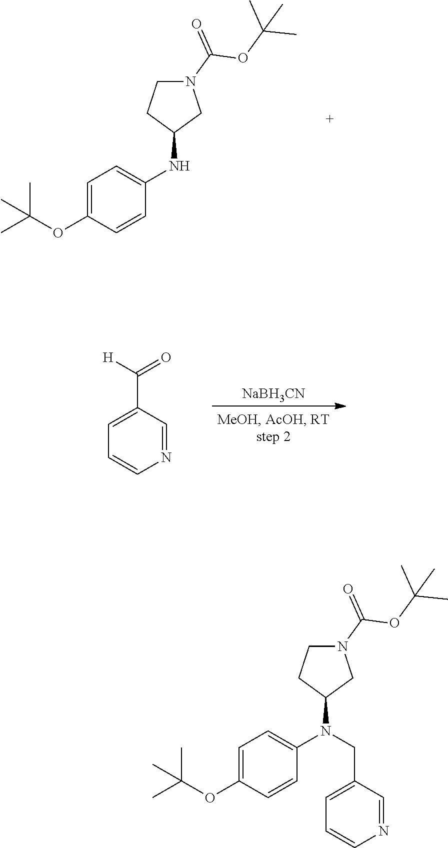

To a stirred solution of tert-butyl (3S)-3-(4-tert-butoxyanilino)pyrrolidine-1-carboxylate (81%, 100 mg, 0.242 mmol) and pyridine-3-carbaldehyde (97%, 47 μL, 0.484 mmol) in methanol (2.5 mL) and acetic acid (300 μL) was added sodium cyanoborohydride (100%, 303 mg, 0.605 mmol) and the reaction mixture was stirred at RT for 18 h then heated-up to 60° C. and stirred for 2 h. The mixture was filtered and washed with MeOH. The filtrate was concentrated under reduced pressure and the residue was diluted with EtOAc and Sat. NaHCO₃. The layers were separated, and the aqueous layer was extracted twice with EtOAc. The combined organic layers were dried over a phase separator and concentrated under reduced pressure. The crude material was purified by flash chromatography (4 g silica, 0-50% EtOAc in heptane eluent). The appropriate fractions were gathered and concentrated under reduced pressure to afford tert-butyl (3S)-3-[4-tert-butoxy-N-(3-pyridylmethyl)anilino]pyrrolidine-1-carboxylate (72 mg, 68%) as a yellow oil. LC-MS: m/z=426 [M+H]⁺.

### Step 3: 4-[3-pyridylmethyl-[(3S)-pyrrolidin-3-yl]amino]phenol

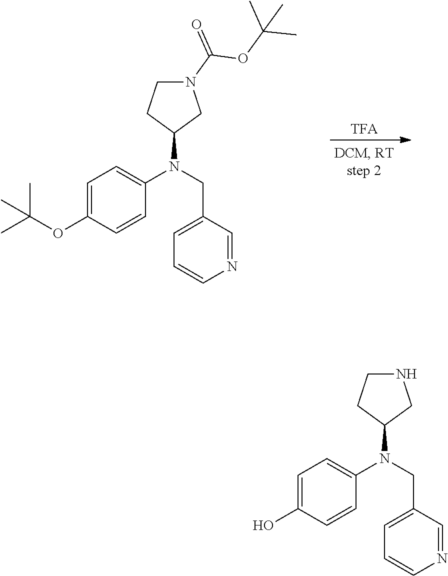 [SMILES: CC(C)(C)OC(=O)N1CC[C@H](N(Cc2cccnc2)c2ccc(OC(C)(C)C)cc2)C1 ; Oc1ccc(N(Cc2cccnc2)[C@H]2CCNC2)cc1 ; "TFA"]

The title compound was synthesized as described in Example 17 (step 2) (N-Boc deprotection) using tert-butyl (3S)-3-[4-tert-butoxy-N-(3-pyridylmethyl)anilino]pyrrolidine-1-carboxylate (68 mg, 0.157 mmol), and was isolated as a light yellow oil (51 mg, Quant.) and was used in next step without more purification. LC-MS: m/z=270 [M+H]⁺.

### Step 4: 4-[[(3S)-1-(1H-indol-6-ylsulfonyl)pyrrolidin-3-yl]-(3-pyridylmethyl)amino]phenol

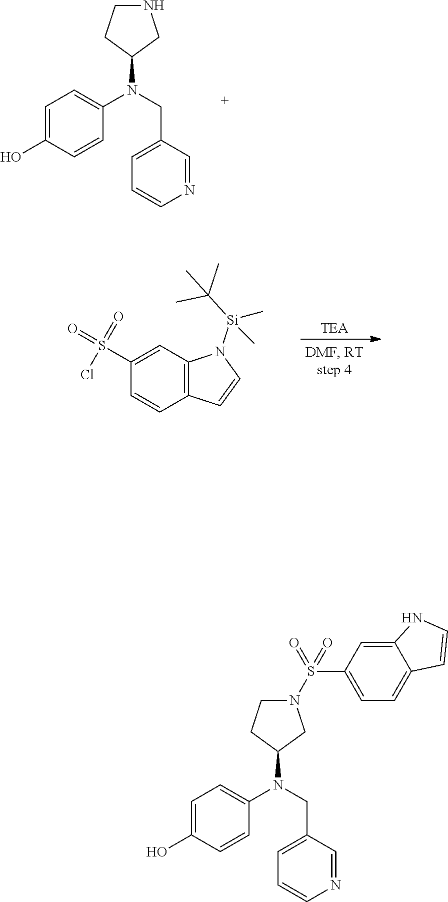 [SMILES: Oc1ccc(N(Cc2cccnc2)[C@H]2CCNC2)cc1 ; CC(C)(C)[Si](C)(C)n1ccc2ccc(S(=O)(=O)Cl)cc21 ; "TEA" ; O=S(=O)(c1ccc2cc[nH]c2c1)N1CC[C@H](N(Cc2cccnc2)c2ccc(O)cc2)C1]

The title compounds were synthesized as described in Example 16 (steps 3 and 4) using 4-[3-pyridylmethyl-[(3S)-pyrrolidin-3-yl]amino]phenol (88%, 51 mg, 0.167 mmol) and 1-triisopropylsilylindole-6-sulfonyl chloride (50 mg, 0.133 mmol), and was isolated as an off-white powder (35 mg, 47%) after purification by reverse-phase flash chromatography (C18, 4 g, 0-100% acetonitrile in water eluent). LC-MS: m/z=449 [M+H]⁺. ¹H NMR (400 MHz, DMSO) δ 11.60 (s, 1H), 8.91 (s, 1H), 8.33 (d, J=4.7 Hz, 1H), 8.22 (s, 1H), 7.85 (s, 1H), 7.76 (d, J=8.3 Hz, 1H), 7.68 (s, 1H), 7.38 (d, J=8.2 Hz, 2H), 7.18 (dd, J=7.9, 4.7 Hz, 1H), 6.66-6.45 (m, 5H), 4.05 (s, 2H), 3.83 (t, J=7.2 Hz, 1H), 3.36 (d, J=10.2 Hz, 2H), 3.09 (q, J=8.4 Hz, 1H), 3.03-2.94 (m, 1H), 1.93 (d, J=8.0 Hz, 1H), 1.63 (dd, J=12.6, 8.1 Hz, 1H).

### Example 51: 4-[7-(1H-indol-6-ylsulfonyl)-3,7-diazabicyclo[4.2.0]octan-3-yl]phenol

### Step 1: tert-butyl 3-(4-tert-butoxyphenyl)-3,7-diazabicyclo[4.2.0]octane-7-carboxylate

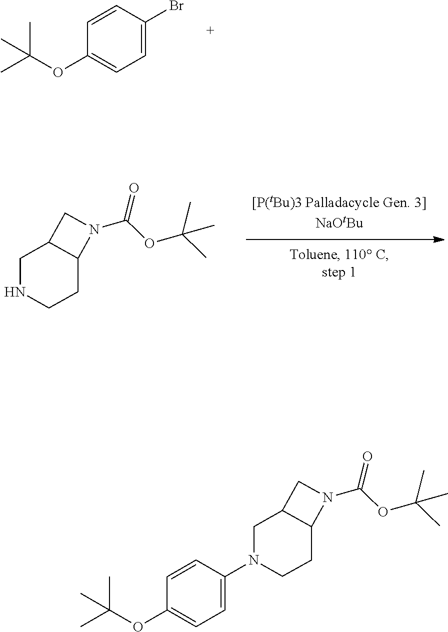 [SMILES: CC(C)(C)Oc1ccc(Br)cc1 ; CC(C)(C)OC(=O)N1CC2CNCCC21 ; CC(C)(C)OC(=O)N1CC2CN(c3ccc(OC(C)(C)C)cc3)CCC21]

The title compound was synthesized from tert-butyl 3,7-diazabicyclo[4.2.0]octane-7-carboxylate (95%, 184 mg, 0.823 mmol) as described in Example 17 (step 1) and was purified through a silica gel column with 0-30% ethyl acetate in heptane to afford the title compound as an orange oil (253 mg, 85%). LC-MS: m/z=361.5 [M+H]⁺.

### Step 2: 4-(3,7-diazabicyclo[4.2.0]octan-3-yl)phenol

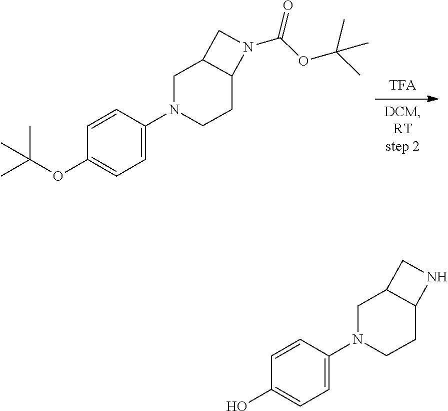 [SMILES: CC(C)(C)OC(=O)N1CC2CN(c3ccc(OC(C)(C)C)cc3)CCC21 ; Oc1ccc(N2CCC3NCC3C2)cc1]

The title compound was synthesized as described in Example 17 (step 2) (N-Boc deprotection) using tert-butyl 3-(4-tert-butoxyphenyl)-3,7-diazabicyclo[4.2.0]octane-7-carboxylate (125 mg, 0.347 mmol) and was isolated as a white powder (140 mg, 100%). LC-MS: m/z=205 [M+H]⁺.

### Step 3: 4-[7-(1H-indol-6-ylsulfonyl)-3,7-diazabicyclo[4.2.0]octan-3-yl]phenol

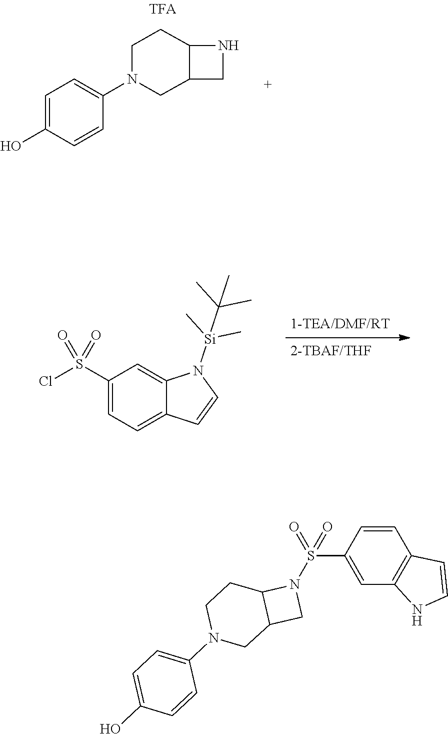 [SMILES: Oc1ccc(N2CCC3NCC3C2)cc1 ; CC(C)(C)[Si](C)(C)n1ccc2ccc(S(=O)(=O)Cl)cc21 ; O=S(=O)(c1ccc2cc[nH]c2c1)N1CC2CN(c3ccc(O)cc3)CCC21]

The title compound was synthesized as described in Example 16 (steps 3 and 4) using with 4-(3,7-diazabicyclo[4.2.0]octan-3-yl)phenol (40 mg, 0.196 mmol) and 1-triisopropylsilylindole-6-sulfonyl chloride (51 mg, 0.137 mmol), and was isolated as a pale pink powder (19.3 mg, 25%) after purification by flash chromatography (silica gel column with 5-70% ethyl acetate in heptane then reverse-phase flash chromatography C18 column, 0-100% ACN in water). LC-MS: m/z=393.8 [M+H]⁺. LC-MS: m/z=384.3 [M+H]⁺. ¹H NMR (400 MHz, DMSO) δ 11.65 (s, 1H), 8.72 (s, 1H), 7.87 (s, 1H), 7.81 (d, J=8.3 Hz, 1H), 7.70 (d, J=3.1 Hz, 1H), 7.40 (dd, J=8.3, 1.6 Hz, 1H), 6.74-6.68 (m, 2H), 6.66-6.59 (m, 3H), 3.90 (dd, J=7.9, 3.6 Hz, 1H), 3.56-3.43 (m, 2H), 3.21 (m, 3H), 3.04 (dd, J=12.9, 7.5 Hz, 1H), 2.42-2.32 (m, 1H), 1.93-1.75 (m, 2H).

### Example A: RNR Enzyme Activity

A rapid-fire mass spectrometry (RF/MS) assay was used to assess RNR enzyme activity using a 384 well plate and a robotic platform.

The plate layout included two validated reference compounds (Triapine (3-AP) and Hydroxyurea (HU)):
A dose response in duplicate, top concentration: 5 μM (3-AP) and 250 μM (HU), semi-log
dilutions.
Spike wells in triplicate randomly spotted at four concentrations:
   250 μM, 100 μM, 30 μM and 2 μM for HU
   5 μM, 2 μM, 0.6 μM and 0.04 μM for 3-AP

First, the multidrop pipes were saturated for 30 minutes with enzymatic solution. Then 30 μL of Stop solution was distributed in column 24. Next, 15 μL of enzyme was distributed in column 1 to 24. Next, a pre-incubation step of 15 minutes at room temperature occurred, followed by distribution of 15 μL of substrate solution (column 1 to 24). Next, the plate was incubated for 45 minutes at 37° C. 30 μL of Stop solution was distributed to columns 1 to 23.

The final parameters for the enzyme reactions were:
Incubation: 37° C., 45 min
[CDP]: 5 μM: [ATP]: 1 mM; [NADPH]: No
[RNR]final: 50 nM with 1:1 (RNR1:RNR2) ratio
Final volume: 30 μL
Stop solution: 6% HCOOH containing 2 μM of 15

The compounds were screened at concentrations up to 50 μM concentrations and the results are shown in table 3.

TABLE 3

| Ex. | RF/MS IC₅₀ μM |
|---|---|
| 1 | A |
| 2 | B |
| 3 | C |
| 4 | C |
| 5 | C |
| 6 | C |
| 7 | B |
| 9 | C |
| 10 | B |
| 11 | A |
| 12 | A |
| 13 | A |
| 14 | A |
| 15 | B |
| 16 | C |
| 17 | A |
| 18 | C |
| 19 | A |
| 20 | A |
| 21 | A |
| 22 | A |
| 23 | A |
| 24 | B |
| 25 | A |
| 26 | C |
| 27 | A |
| 28 | A |
| 29 | A |
| 30 | A |
| 31 | A |
| 34 | A |
| 35 | C |
| 36 | C |
| 37 | A |
| 38 | A |
| 39 | A |
| 40 | A |
| 41 | C |
| 43 | A |
| 44 | B |
| 45 | B |
| 46 | B |
| 47 | C |
| 50 | C |
| 51 | C |

RF/MS IC₅₀
A: IC₅₀ ≤ 50 nM;
B: 50 nM < IC₅₀ ≤ 100 nM
C: 100 nm < IC₅₀ ≤ 500 nM
D: 500 nM < IC₅₀ ≤ 1 μM
E: 1 μM < IC₅₀ ≤ 10 μM
NT: not tested

### Example B: Alphalisa Assay

Colo320 DM cells (ATCC #CCL-220, derived from human colorectal adenocarcinoma, Dukes' type C) were seeded on a 96-well, cell culture treated assay plate at a density of 50,000 cells/well in 200 μL of RPMI-1640 media supplemented with 10% Fetal Bovine Serum and incubated at 37° C. overnight. The following day, test compound dilutions were added directly to the plated cells by a Tecan digital dispenser to a final DMSO concentration of <0.5%, and incubated at 37° C. overnight (approximately 16 hours). The following day all cull culture media was removed from the cells. 75 μL of 1× AlphaLisa lysis buffer was added to each well and plates were agitated on a shaker for 30 minutes at room temperature. The lysis of cells and detection of pCHK1 (S345) were performed with regents contained within the AlphaLisa Sure Fire assay kit (Perkin Elmer #ALSU-PCHK1-A) according to the manufacturer's instructions. 10 μL of each lysate was then transferred to a white, 384-well assay plate (Perkin Elmer #6008280), 5 μL of Acceptor mix was then added to each well of lysate in the white, 384-well assay plate and incubated in the dark at room temperature for 60 minutes. 5 μL of Donor mix was then added to each well of the white, 384-well assay plate in subdued light and incubated at room temperature for 60 minutes. Plates were read on an Alpha Technology-compatible plate reader using standard AlphaLisa settings.

The results are shown in table 4.

TABLE 4

| Ex. | Alphalisa - IC₅₀ μM |
|---|---|
| 1 | C |
| 2 | B |
| 3 | C |
| 4 | C |
| 5 | B |
| 6 | D |
| 7 | B |
| 8 | B |
| 9 | C |
| 10 | B |
| 11 | B |
| 12 | C |
| 13 | A |
| 14 | C |
| 15 | B |
| 16 | B |
| 17 | A |
| 18 | D |
| 19 | B |
| 20 | A |
| 21 | B |
| 22 | B |
| 23 | B |
| 24 | B |
| 25 | A |
| 26 | C |
| 27 | A |
| 28 | B |
| 29 | C |
| 30 | A |
| 31 | B |
| 32 | B |
| 33 | B |
| 34 | B |
| 35 | D |
| 36 | B |
| 37 | B |
| 38 | B |
| 39 | B |
| 40 | A |
| 41 | D |
| 42 | B |
| 43 | B |
| 44 | C |
| 45 | C |
| 46 | B |
| 47 | D |
| 48 | B |
| 49 | B |
| 50 | D |
| 51 | C |

A: IC₅₀ ≤ 1 μM;
B: 1 μM < IC₅₀ ≤ 5 μM
C: 5 μm < IC₅₀ ≤ 10 μM
D: 10 μM < IC₅₀ ≤ 50 μM
E: 50 μM < IC₅₀
NT: not tested

### Example C: Pharmaceutical Compositions

### Example C1: Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example C₂: Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example C₃: Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

The examples and embodiments described herein are for illustrative purposes only and in some embodiments, various modifications or changes are to be included within the purview of disclosure and scope of the appended claims.

1. A compound of Formula (I), or a pharmaceutically acceptable salt, solvate, or stereoisomer thereof: 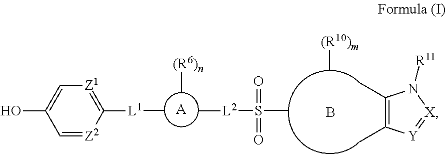
wherein:
Z¹ is N or CR¹;
R¹ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Z² is N or CR²;
R² is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent, —C(R³R⁴)—, —O—, —S—, —NR⁵—, —C(R³R⁴)—O—, —O—C(R³R⁴)—, —C(R³R⁴)—S—, —S—C(R³R⁴)—, —C(R³R⁴)—NR⁵—, or —NR⁵—C(R³R⁴)—;
R³ and R⁴ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁵ is hydrogen, —S(═O)R^{a}, —S(═O)₂R^{a}, —S(═O)₂NR^{c}R^{d}, —C(═O)R, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl);
Ring A is cycloalkyl, heterocycloalkyl, aryl, or heteroaryl;
each R⁶ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
n is 0, 1, 2, 3, or 4;
L² is absent, —CR⁷R⁸—, or —NR⁹-R⁷ and R⁸ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁹ is hydrogen, —S(═O)R^{a}, —S(═O)₂R^{a}, —S(═O)₂NR^{c}R^{d}, —C(═O)R, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl);
Ring B is phenyl or a 6-membered heteroaryl;
each R¹⁰ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
m is 0, 1, 2, or 3;
R¹¹ is hydrogen, —S(═O)R^{a}, —S(═O)₂R, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X is N or CR^{X};
R^{X} is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is N or CR^{Y};
R^{Y} is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 3- to 6-membered cycloalkyl, or 3- to 6-membered heterocycloalkyl;
or two R on the same atom are taken together to form an oxo;
provided that the compound is not:

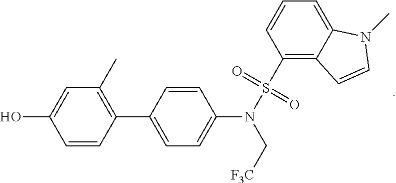 [SMILES: Cc1cc(O)ccc1-c1ccc(N(CC(F)(F)F)S(=O)(=O)c2cccc3c2ccn3C)cc1]

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. The compound of claim 1, or a pharmaceutically acceptable salt, solvate, or stereoisomer thereof, wherein the compound of Formula (I) is of Formula (Ia) or Formula (Ib): 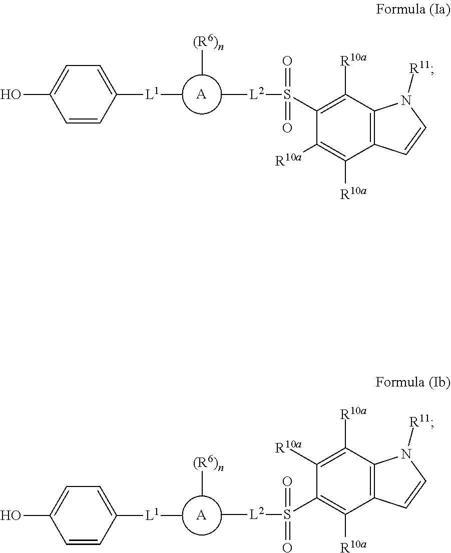
wherein each R^{10A} is independently hydrogen or R¹⁰.

20. (canceled)

21. (canceled)

22. (canceled)

23. (canceled)

24. (canceled)

25. The compound of claim 19, or a pharmaceutically acceptable salt, solvate, or stereoisomer thereof, wherein each R^{10A} is hydrogen.

26. (canceled)

27. The compound of claim 19, or a pharmaceutically acceptable salt, solvate, or stereoisomer thereof, wherein L¹ is absent, —O—, —NR⁵—, or —NR⁵—C(R³R⁴)—.

28. The compound of claim 27, or a pharmaceutically acceptable salt, solvate, or stereoisomer thereof, wherein L¹ is absent.

29. (canceled)

30. The compound of claim 27, or a pharmaceutically acceptable salt, solvate, or stereoisomer thereof, wherein L¹ is —NR⁵—.

31. (canceled)

32. (canceled)

33. (canceled)

34. (canceled)

35. The compound of claim 30, or a pharmaceutically acceptable salt, solvate, or stereoisomer thereof, wherein R⁵ is hydrogen or C₁-C₆alkyl.

36. (canceled)

37. (canceled)

38. The compound of claim 19, or a pharmaceutically acceptable salt, solvate, or stereoisomer thereof, wherein L² is absent.

39. The compound of claim 19, or a pharmaceutically acceptable salt, solvate, or stereoisomer thereof, wherein L² is NR⁹—.

40. The compound of claim 39, or a pharmaceutically acceptable salt, solvate, or stereoisomer thereof, wherein R⁹ is hydrogen or C₁-C₆alkyl.

41. (canceled)

42. The compound of claim 1, or a pharmaceutically acceptable salt, solvate, or stereoisomer thereof, wherein R¹¹ is hydrogen.

43. (canceled)

44. (canceled)

45. (canceled)

46. (canceled)

47. The compound of claim 19, or a pharmaceutically acceptable salt, solvate, or stereoisomer thereof, wherein Ring A is 4- to 6-membered monocyclic heterocycloalkyl.

48. (canceled)

49. (canceled)

50. (canceled)

51. The compound of claim 19, or a pharmaceutically acceptable salt, solvate, or stereoisomer thereof, wherein Ring A is

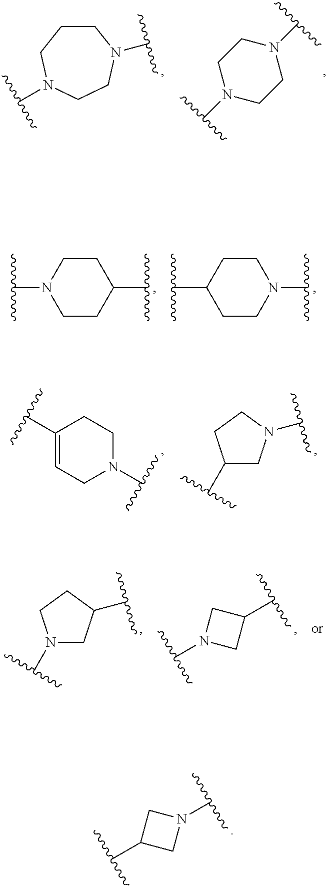

52. (canceled)

53. (canceled)

54. The compound of claim 19, or a pharmaceutically acceptable salt, solvate, or stereoisomer thereof, wherein Ring A is 8- to 10-membered bicyclic heterocycloalkyl.

55. (canceled)

56. (canceled)

57. (canceled)

58. The compound of claim 19, or a pharmaceutically acceptable salt, solvate, or stereoisomer thereof, wherein Ring A is

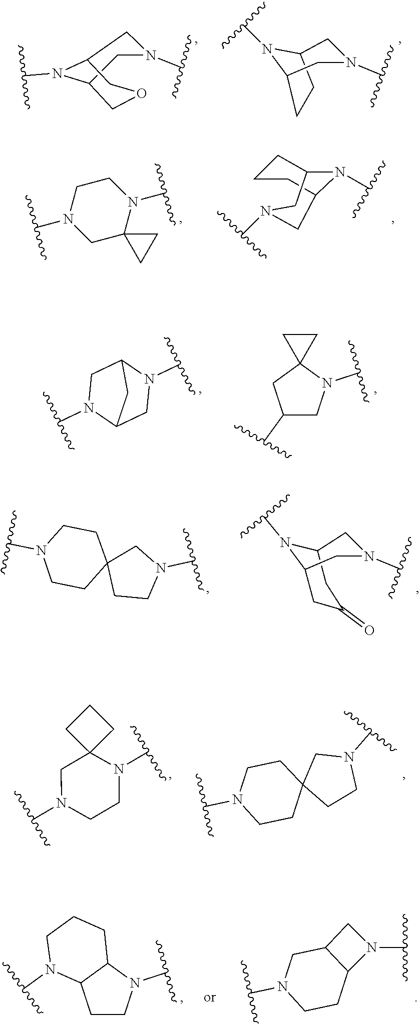

59. (canceled)

60. The compound of claim 19, or a pharmaceutically acceptable salt, solvate, or stereoisomer thereof, wherein each R⁶ is independently halogen or C₁-C₆alkyl and n is 0 or 1.

61. (canceled)

62. (canceled)

63. A compound selected from the group consisting of: 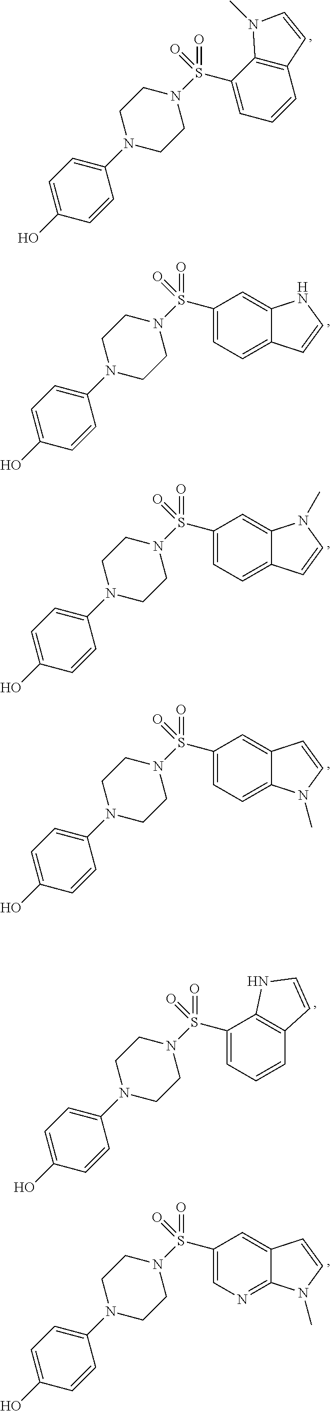 [SMILES: Cn1ccc2cccc(S(=O)(=O)N3CCN(c4ccc(O)cc4)CC3)c21 ; O=S(=O)(c1ccc2cc[nH]c2c1)N1CCN(c2ccc(O)cc2)CC1 ; Cn1ccc2ccc(S(=O)(=O)N3CCN(c4ccc(O)cc4)CC3)cc21 ; Cn1ccc2cc(S(=O)(=O)N3CCN(c4ccc(O)cc4)CC3)ccc21 ; O=S(=O)(c1cccc2cc[nH]c12)N1CCN(c2ccc(O)cc2)CC1 ; Cn1ccc2cc(S(=O)(=O)N3CCN(c4ccc(O)cc4)CC3)cnc21] 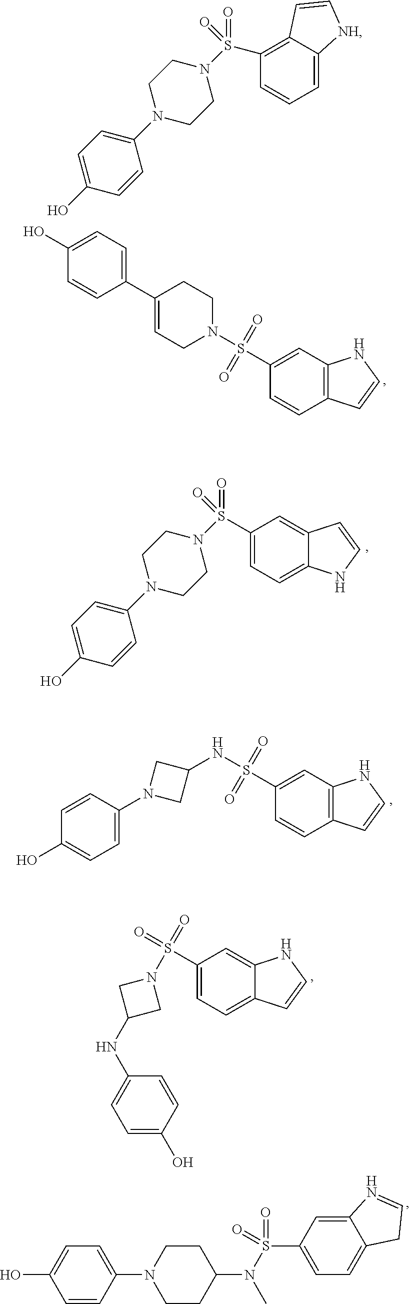 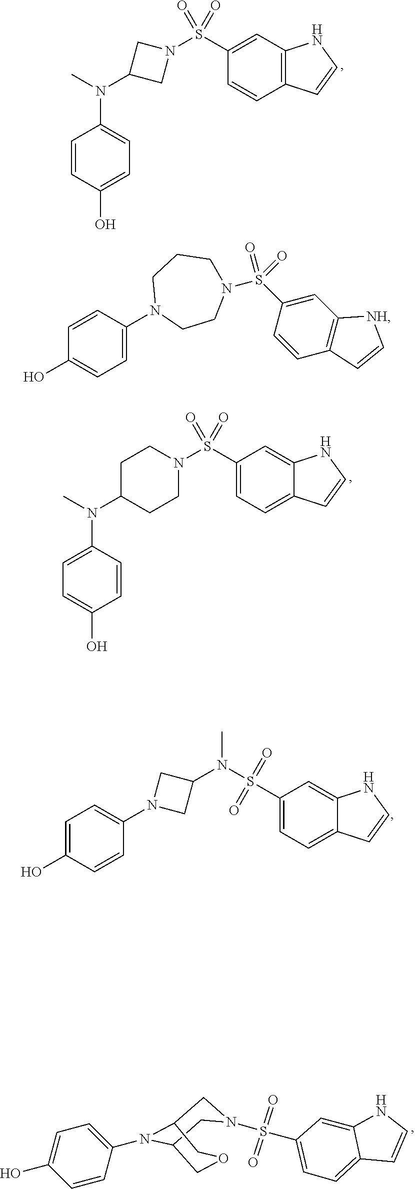 [SMILES: CN(c1ccc(O)cc1)C1CN(S(=O)(=O)c2ccc3cc[nH]c3c2)C1 ; O=S(=O)(c1ccc2cc[nH]c2c1)N1CCCN(c2ccc(O)cc2)CC1 ; CN(c1ccc(O)cc1)C1CCN(S(=O)(=O)c2ccc3cc[nH]c3c2)CC1 ; CN(C1CN(c2ccc(O)cc2)C1)S(=O)(=O)c1ccc2cc[nH]c2c1 ; O=S(=O)(c1ccc2cc[nH]c2c1)N1CC2COCC(C1)N2c1ccc(O)cc1] 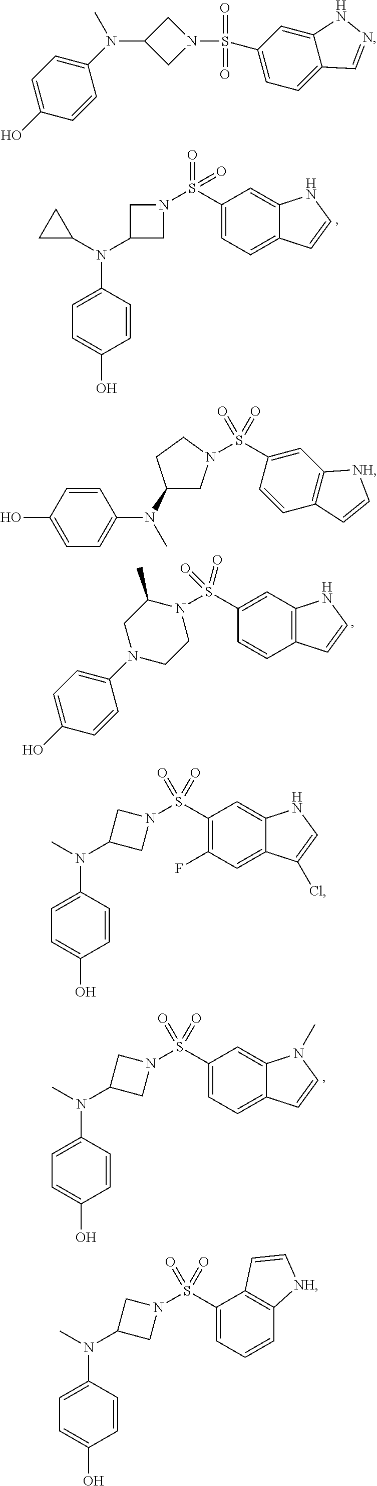 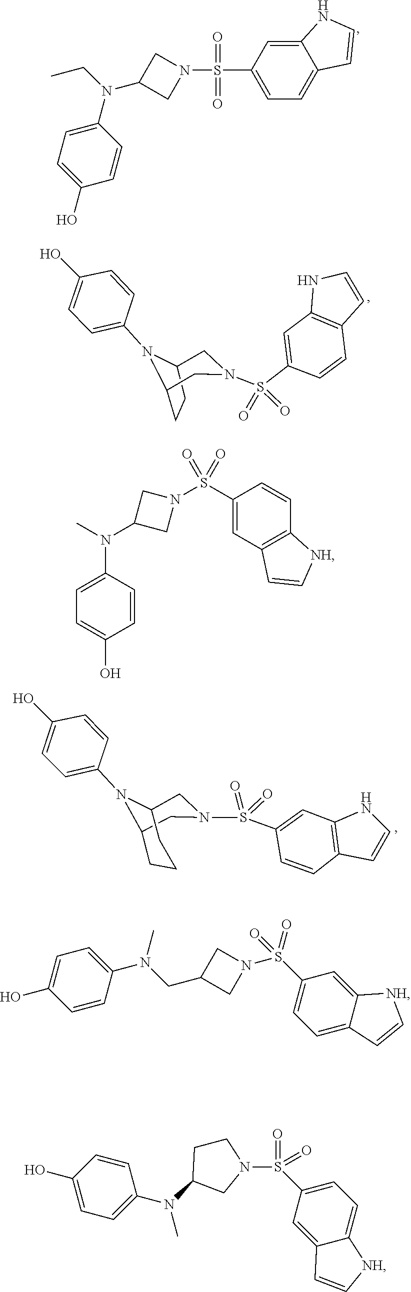 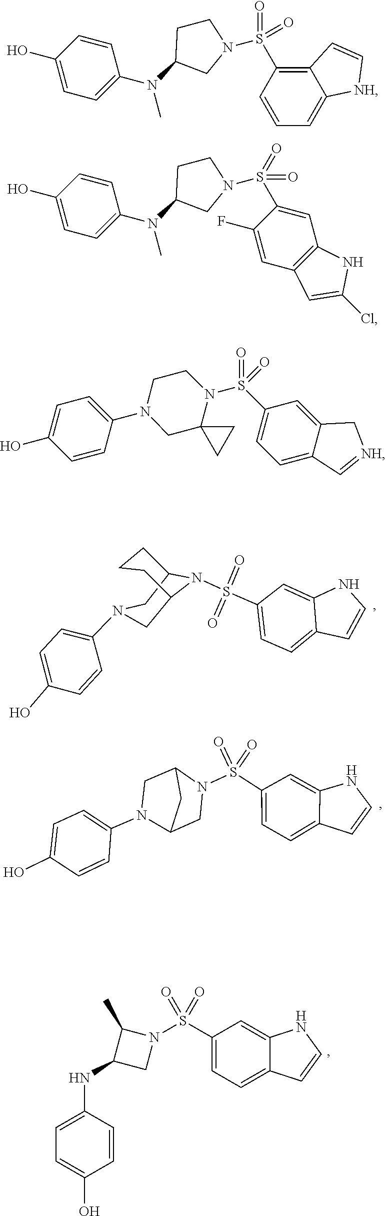 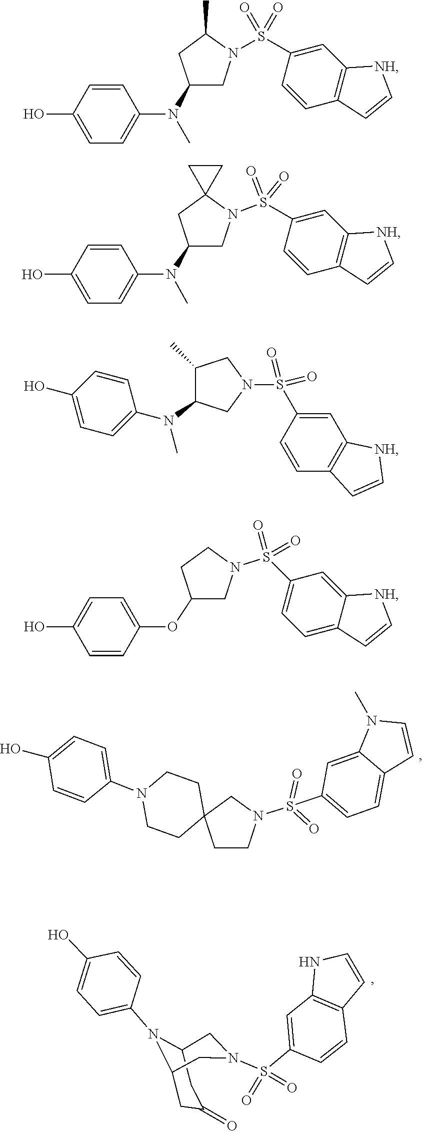 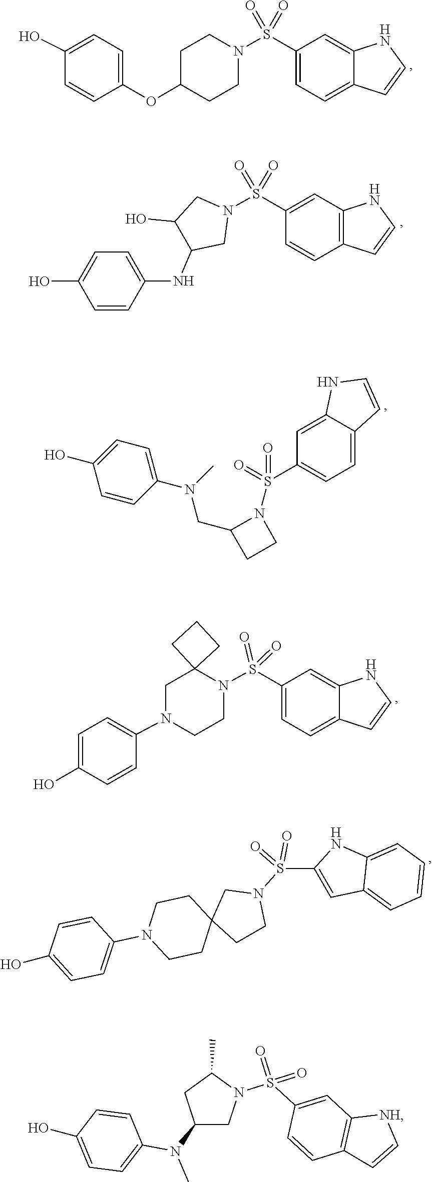 [SMILES: O=S(=O)(c1ccc2cc[nH]c2c1)N1CCC(Oc2ccc(O)cc2)CC1 ; O=S(=O)(c1ccc2cc[nH]c2c1)N1CC(O)C(Nc2ccc(O)cc2)C1 ; CN(CC1CCN1S(=O)(=O)c1ccc2cc[nH]c2c1)c1ccc(O)cc1 ; O=S(=O)(c1ccc2cc[nH]c2c1)N1CCN(c2ccc(O)cc2)CC12CCC2 ; O=S(=O)(c1cc2ccccc2[nH]1)N1CCC2(CCN(c3ccc(O)cc3)CC2)C1 ; C[C@H]1C[C@H](N(C)c2ccc(O)cc2)CN1S(=O)(=O)c1ccc2cc[nH]c2c1] 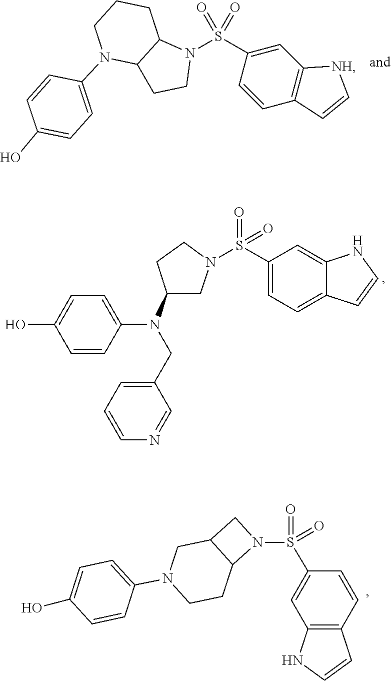 [SMILES: O=S(=O)(c1ccc2cc[nH]c2c1)N1CCC2C1CCCN2c1ccc(O)cc1 ; O=S(=O)(c1ccc2cc[nH]c2c1)N1CC[C@H](N(Cc2cccnc2)c2ccc(O)cc2)C1 ; O=S(=O)(c1ccc2cc[nH]c2c1)N1CC2CN(c3ccc(O)cc3)CCC21]
or a pharmaceutically acceptable salt solvate, or stereoisomer thereof.

64. A pharmaceutical composition comprising a compound of claim 1, or a pharmaceutically acceptable salt, solvate, or stereoisomer thereof, and a pharmaceutically acceptable excipient.

65. A method of treating cancer in a subject, comprising administering to the subject a compound of claim 1, or a pharmaceutically acceptable salt, solvate, or stereoisomer thereof.

66. (canceled)

67. (canceled)

68. (canceled)

69. A method of treating an ecDNA-associated tumor or tumor cells comprising administering a compound of claim 1, or a pharmaceutically acceptable salt, solvate, or stereoisomer thereof, to a subject identified as having a tumor or tumor cells having ecDNA, wherein growth or size of the tumor or growth or number of the tumor cells is decreased as a result of treatment: wherein the method further comprises administering a cancer-targeted therapeutic agent.

70. (canceled)

71. (canceled)

72. (canceled)
